(19) United States
(12) Patent Application Publication
(10) Pub. No.: US 2025/0250380 A1
(43) Pub. Date: Aug. 7, 2025

(54) AMINE CONTAINING POLYMERS AND LUBRICANT COMPOSITIONS INCLUDING SUCH POLYMERS AND METHODS OF USING THEREOF

(71) Applicant: INFINEUM INTERNATIONAL LIMITED, LINDEN, NJ (US)

(72) Inventors: Evan Haidasz, HOBOKEN, NJ (US); Marianne Stol, Haarlem, Noord-Holland (NL)

(21) Appl. No.: 18/791,876

(22) Filed: Aug. 1, 2024

Related U.S. Application Data
(63) Continuation-in-part of application No. 18/411,304, filed on Jan. 12, 2024.
(60) Provisional application No. 63/549,831, filed on Feb. 5, 2024.

(51) Int. Cl. C08F 236/08; C07C 211/28; C08F 8/04; C08F 212/08; C08L 53/00; C10M 143/10; C10M 143/12; C10M 169/04; C10N 20/00; C10N 20/04; C10N 30/02; C10N 30/04; C10N 40/25; C10N 70/00

(52) U.S. Cl. CPC C08F 236/08; C07C 211/28; C08F 8/04; C08F 212/08; C08L 53/00; C10M 143/10; C10M 143/12; C10M 169/04; C10N 2020/04; C10N 2020/073; C10N 2030/02; C10N 2030/04; C10N 2040/252; C10N 2070/00

(57) ABSTRACT

Provided are functional polymers based on functionalized styrenic monomers including a nitrogen-containing moiety, other than as pendant to the phenyl ring that are polymerized from addition-polymerizable monomer compositions including amine-derivatized alpha-methyl styrene (ADAMS) monomers according to structure (I).

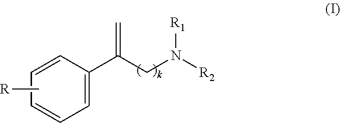

wherein k is an integer from 1 to 3, preferably 2; wherein R₁ is a methyl group or a phenyl group and R₂ is a benzyl group, or wherein R₁ and R₂ are connected to form a moiety containing one 6-membered ring, with four carbons, bearing an O or N—CH₃ group at the 4-position of the 6-membered ring; wherein R, in structure (I), is hydrogen, a phenyl ring co-attached at two neighboring ring carbon positions with the phenyl ring shown so as to form a naphthalene assembly, a phenyl group attached at a single carbon of the phenyl ring shown, a C₁-C₄ hydrocarbyl group (such as a methyl group), a C₁-C₆ hydrocarbyl group containing 1 to 4 additional heteroatoms (such as O, N, S, P, Se, and combinations thereof). Also provided are methods of using such functional polymers in lubricating oil compositions.

### CROSS-REFERENCE TO RELATED APPLICATION

This U.S. Non-provisional application claims priority to U.S. Provisional Application Ser. No. 63/549,831 filed on Feb. 5, 2024, the contents of which are herein incorporated by reference in their entirety, and also claims priority to U.S. Non-Provisional application Ser. No. 18/411,304 filed on Jan. 12, 2024, the contents of which are herein incorporated by reference in their entirety, which claims priority to U.S. Provisional Ser. No. 63/483,365 filed on Feb. 6, 2023, the contents of which are also herein incorporated by reference in their entirety.

### FIELD

This disclosure relates to polymer compositions and methods of using such compositions, which may be derived from monomer compositions containing aromatic and/or conjugated (non-aromatic) structures each containing at least one aminic nitrogen. In particular, such functional monomers may be anionically polymerized to form the functional polymers disclosed herein. This disclosure also relates to the use of such polymer compositions as additives in lubricant compositions having good dispersancy in engine crankcase applications, especially in compression ignited engine applications.

### BACKGROUND

There are myriad references disclosing nitrogen-containing (amine) groups pendant to the phenyl ring in styrenic monomers/polymers/copolymers. However, monomers and polymers bearing such pendant groups can be chemically difficult to synthesize reliably/repetitively. Even when such synthesis can be accomplished, there can be issues with propagation within the polymerization reactions containing such amine-functionalized styrenic monomers, whether alone or as copolymer with other styrenic monomers.

Thus, frequently the amine group can be added to a smattering of monomer repeat units using post-polymerization chemical reactions. But post-polymerization chemistry can often come through with different issues, even if it may avoid the propagation issues of amine-functionalized styrenic monomers.

As such, it is desirable to develop an unconventional way to functionalize a styrenic monomer pre-polymerization, particularly one that was neutral to, or perhaps even enhanced, the propagation within the polymerization reaction.

U.S. Pat. Nos. 6,486,272, 9,364,825, 10,202,494, and 10,046,285, disclose polymers made from styrenic monomers bearing nitrogen-containing groups pendant to the phenyl ring, all of which are herein incorporated by reference in their entirety. GB Patent No. 1 381 755 discloses amine-functional monomer compounds, but only with acrylamide functionality. Other examples of potentially relevant publications include, but are not necessarily limited to, U.S. Pat. Nos. 7,790,661, 7,960,320, 8,778,854, and 10,414,999; and PCT Publication No. WO 2021/127183, all of which are herein incorporated by reference in their entirety.

Based on the difficulty of preparing and polymerizing styrenic monomers bearing nitrogen-containing groups pendant to the phenyl ring, Applicant has explored other potential structures for functional monomers which are both simpler to manufacture and also polymerize. These functional monomer compositions containing aromatic and/or conjugated (non-aromatic) structures each containing at least one aminic nitrogen are described in commonly owned related U.S. Provisional Application Ser. No. 63/483,365 filed on Feb. 6, 2023, the contents of which are herein incorporated by reference in their entirety.

U.S. Pat. No. 2,778,826 (“the '826 Schmidle patent”) and a 1955 article by Schmidle and Mansfield, entitled “The Aminomethylation of Olefins. I. The Reaction of Secondary Amines, Formaldehyde, and Olefins,” both disclose various reactions alleging formation of 3-aryl-3-butenyl-1-amines, in which the formaldehyde and secondary amine allegedly formed an iminium, which reacted with the styrenic olefin to form only the terminal (vinylidene) double-bond version of the amine-functional styrenic. The 1955 article also disclosed amine-functionalization of terpenoids such as α- and β-pinene, camphene, and limonene, but not isoprene or similar conjugated non-aromatic compounds.

1983 article by Cohen and Onopchenko, entitled “Competing Hydride Transfer and Ene Reactions in the Aminoalkylation of 1-Alkenes with N,N-Dimethylmethyleniminium Ions. A Literature Correction” (citing, in part, to the '826 Schmidle patent, inter alia), further disclosed a mechanistic study of specifically dimethyliminimum compounds reacting with styrenic and non-styrenic olefins. Notably, at the beginning of the Discussion section, the 1983 article opines that the '826 Schmidle patent (and presumably the 1955 article containing strikingly similar experiments and results thereto) had made mistakes. Nonetheless, with respect to aminomethylation of α-methylstyrene, the 1983 article indicated formation of vinylidene-based product, in tandem with a significant vinylene (not terminal double-bond) content and a quite significant (13%, in the case of the dimethylamino-version) saturated arylalkane-amine content.

The inventive monomers disclosed in U.S. Provisional Application Ser. No. 63/483,365 have, to the best of the Applicant's knowledge, not been previously polymerized.

Based on the foregoing, there is a need to provide functional polymers based on alpha-substituted functional monomers, and in particular functional polymers based on functional monomer compositions containing aromatic and/or conjugated (non-aromatic) structures each containing at least one aminic nitrogen utilizing anionic polymerization processing techniques.

The emphasis on fuel economy has been increased in recent years. One approach to improve the fuel economy of vehicles is to design new lubricant oils that reduce friction while maintaining a good film thickness for durability and wear protection while also preventing soot-induced viscosity increase. In an attempt to improve fuel economy, the use and stipulation of low viscosity grades by Original Equipment Manufacturers (OEM) is becoming increasingly widespread. One of the challenges for the provision of engine and/or drive train transmission oils having these reduced viscosity grades is maintaining cleanliness. Such oils must be able to reduce sludge, provide good soot handling, and provide wear protection, whilst providing desired fuel economy benefits. These targets should be achieved while maintaining low levels of sulphated ash and phosphorus, as well as ensuring seal compatibility. There is a need to provide new engine oils having low viscosity grades that meet these requirements.

Base oils for lubricants are typically modified by the addition of additives such as viscosity index improvers (VIIs) and/or dispersants. VIIs may be used to reduce the extent to which the viscosity of lubricants changes with temperature and are often used to formulate engine and transmission lubricants. Common VIIs typically include polymeric materials that may be derived from ethylene-propylene copolymers, polymethacrylates, hydrogenated styrene-butadiene copolymers, polyisobutylenes, etc.

During engine operation, oil-insoluble oxidation byproducts, such as soot, are produced. Dispersants help keep these byproducts suspended or in solution, thus diminishing their deposition on metal surfaces. Common dispersants include (poly)alkenylsuccinic derivatives, such as hydrocarbyl-substituted succinic anhydrides, such as polyisobutylene succinic anhydride (PIBSA) and hydrocarbyl-substituted succinimides, such as polyisobutylene succinimides (PIBSA-PAM), such as those derived from reaction of maleated polyisobutylene with N-phenyl-p-phenylenediamine. Useful dispersants include polyisobutenes, which have been modified by the ene reaction to include functional groups, such as, succinimides, hydroxyethyl imides, succinate esters/amides, and oxazolines. Other dispersants include Mannich base derivatives of polybutenes, ethylene propylene polymers, and acrylic polymers.

Other dispersants are derived from the reaction of maleated polyalphaolefins (such as ethylene-propylene copolymers) and polyamine. U.S. Pat. No. 6,107,257 relates to additives for lubricating oil compositions that comprise multi-functional olefin copolymer viscosity index improvers. Maleic anhydride is reacted or grafted onto an ethylene-propylene copolymer backbone in the presence of a solvent and then the grafted copolymer is reacted with a polyamine such as an N-arylphenylene diamine in the presence of a surfactant to provide a multi-functional olefin copolymer viscosity index improver. Similarly, U.S. Pat. No. 6,107,258 relates to multifunctional fuel and lubricant additives derived from acylated then aminated copolymers of C3 to C23 alpha olefins.

Still other dispersants are derived from styrenic copolymers. U.S. Pat. No. 6,248,702 discloses maleated selectively hydrogenated styrenic block copolymer (Mn 10,000, Ex 1.) reacted with aminopropyl morpholine to form a dispersant substance.

Still other dispersants are derived from copolymers of two different conjugated dienes, such as block copolymers of isoprene and butadiene. U.S. Pat. No. 5,780,540 discloses functionalized selectively hydrogenated isoprene butadiene di-block copolymers in automotive additive packages. Examples show N-phenyl-1,4-phenylenediamine used in combination with polyethylene glycol monoalcohol, 4-(3-aminopropyl morpholine) and/or 3-dibutylaminopropylamine to functionalize maleated 10,000 Mn and/or 20,000 Mn isoprene-butadiene copolymers. Examples 1 and 2 show the IB copolymer is made with 2,2′ dipyridyl, which makes it less likely to have high 1,4 insertions.

Similarly, U.S. Pat. No. 6,319,881 discloses functionalized selectively hydrogenated isoprene butadiene di-block copolymers in automotive additive packages. Example IV shows a maleated selectively hydrogenated isoprene butadiene di-block copolymer (Mn 15,000) that is reacted with aminopropyl morpholine to form a morpholinopropyl succinimide adduct, that is then used as a dispersant in an additive package (Example V).

U.S. Pat. No. 5,073,600 relates to a reactive extrusion process to functionalize (for example, maleate then aminate) copolymers of conjugated diolefins typically having Mn's of 500,000 to about 3,000,000. The examples show a hydrogenated “low” molecular weight star polymer of hydrogenated homo-polyisoprene with an average of 15 arms (35,000 Mn per arm) reacted with maleic anhydride and diethylaminopropylemine in a reactive extruder.

There remains a need to provide alternative or improved engine/transmission oil compositions that meet stringent wear tests, while also providing improved wear, fuel economy and dispersancy performance. The present disclosure provides engine oil compositions including amine containing copolymers that reduce wear, as well as having acceptable soot handling, and/or engine/transmission cleanliness. Further, this disclosure addresses these needs by providing amine containing copolymers as dispersant viscosity index improvers and their use in lubricating oil compositions to achieve the aforementioned performance improvement needs.

### SUMMARY

In one form, disclosed herein is a copolymer comprising:
(a) 10.0 to 20.0 wt % of amine-derivatized alpha-methyl styrene (ADAMS) repeat units according to structure (I):

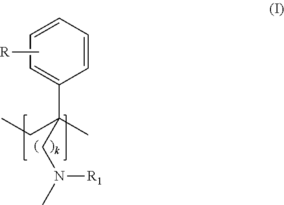

wherein:
k is an integer from 1 to 3;
R₁ a hydrogen or a benzyl group
R is hydrogen, a phenyl ring co-attached at two neighboring ring carbon positions with the phenyl ring shown so as to form a naphthalene assembly, a phenyl group attached at a single carbon of the phenyl ring shown, a C1-C4 hydrocarbyl group, a C1-C6 hydrocarbyl group containing 1 to 4 additional heteroatoms selected from the group consisting of O, N, S, P, Se, and combinations thereof, and
(b) 80.0 to 90.0 wt % of repeat units corresponding to the reacted form of isoprene, and wherein the peak-average molecular weight of the copolymer is between 45.0 to 65.0 kDa.

In another form, disclosed herein is a copolymer comprising:
(a) 5.0 to 10.0 wt % of amine-derivatized alpha-methyl styrene (ADAMS) repeat units according to structure (II):

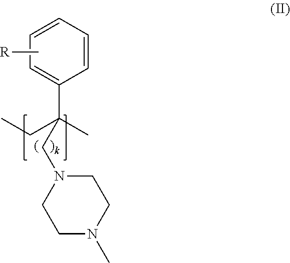

wherein:
k is an integer from 1 to 3;
R is hydrogen, a phenyl ring co-attached at two neighboring ring carbon positions with the phenyl ring shown so as to form a naphthalene assembly, a phenyl group attached at a single carbon of the phenyl ring shown, a C1-C4 hydrocarbyl group, a C1-C6 hydrocarbyl group containing 1 to 4 additional heteroatoms selected from the group consisting of O, N, S, P, Se, and combinations thereof, and
(b) the remaining 90.0 to 95.0 wt % of repeat units corresponding to the reacted form of isoprene, and wherein, the peak-average molecular weight of the copolymer is between 24.0 to 42.0 kDa.

In yet another form, disclosed herein is a copolymer comprising:
(a) 4.0 to 6.0 wt % of amine-derivatized alpha-methyl styrene (ADAMS) repeat units according to structure (III):

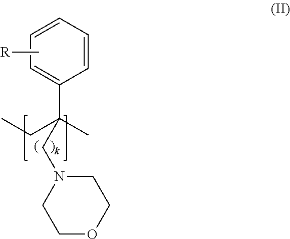

wherein:
k is an integer from 1 to 3;
R is hydrogen, a phenyl ring co-attached at two neighboring ring carbon positions with the phenyl ring shown so as to form a naphthalene assembly, a phenyl group attached at a single carbon of the phenyl ring shown, a C1-C4 hydrocarbyl group, a C1-C6 hydrocarbyl group containing 1 to 4 additional heteroatoms selected from the group consisting of O, N, S, P, Se, and combinations thereof,
(b) the remaining 94.0 to 96.0 wt % of repeat units corresponding to the reacted form of isoprene, and wherein, the peak-average molecular weight of the copolymer is between 36.0 to 46.0 kDa.

In still yet another form, disclosed herein is a copolymer comprising:
(a) one or more amine-derivatized alpha-methyl styrene (ADAMS) repeat units according to structure (IV):

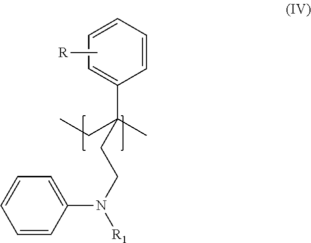

wherein:
k is an integer from 1 to 3;
R₁ a hydrogen or a benzyl group
R is hydrogen, a phenyl ring co-attached at two neighboring ring carbon positions with the phenyl ring shown so as to form a naphthalene assembly, a phenyl group attached at a single carbon of the phenyl ring shown, a C1-C4 hydrocarbyl group, a C1-C6 hydrocarbyl group containing 1 to 4 additional heteroatoms selected from the group consisting of O, N, S, P, Se, and combinations thereof, and
(b) the remaining repeat units corresponding to the reacted form of isoprene.

Also provided herein are lubricating oil composition comprising or resulting from the admixing of: (i) at least 50 wt % of one or more base oils, based upon the weight of the lubricating oil composition; (ii) one or more dispersants; (iii) one or more detergents; and (iv) one or more copolymers of any one of the preceding four paragraphs.

Also provided herein are lubricating oil composition comprising or resulting from the admixing of: (i) from 50 to 99 mass % of the one or more of the base oils, based upon the weight of the lubricating oil composition; (ii) from 0.01 to 20 wt % based on total weight of the lubricating oil composition, of the one or more dispersants; (iii) from 0.10 to 20 mass %, based upon the weight of the lubricating oil composition, of the one or more detergents; and (iv) from 0.10 to 20 mass %, based upon the weight of the lubricating oil composition, of the one or more copolymers described in the preceding paragraphs.

Also provided herein is a method of lubricating an internal combustion engine during operation of the engine comprising: (i) providing to a crankcase of the internal combustion engine the lubricating oil composition of any one of the two preceding paragraphs; (ii) providing a fuel in the internal combustion engine; and (iii) combusting the fuel in the internal combustion engine.

Other aspects of the present disclosure may become apparent from the Detailed Description and Examples sections hereinbelow.

### DETAILED DESCRIPTION

All numerical values within the detailed description and the claims herein are modified by “about” or “approximately” the indicated value, and take into account experimental error and variations that would be expected by a person having ordinary skill in the art.

### Overview of Copolymers

The present disclosure provides for novel polymers and copolymers based on anionic polymerization of functionalized styrenic monomers including a nitrogen-containing moiety, other than as pendant to the phenyl ring. Thus, the monomers of structure (I) were developed to achieve nitrogen-containing functionality on an alpha-substituted styrenic monomer other than as pendant to the phenyl ring on the styrene unit.

It should be noted that prior art references often describe functionalized styrenic monomer with nitrogen containing groups pendant to the phenyl ring in general terms (e.g. “dimethylaminoethyl styrene”), which can be similar to the k=2 monomer structure provided below, however, the prior art does not teach or suggest the alpha-substituted functional monomers or the functional polymers derived therefrom, which are specifically disclosed herein.

The inventive functional polymers disclosed herein based on functionalized styrenic monomers including a nitrogen-containing moiety, other than as pendant to the phenyl ring may be polymerized from addition-polymerizable monomer compositions including amine-derivatized alpha-methyl styrene (ADAMS) monomers according to structure (I).

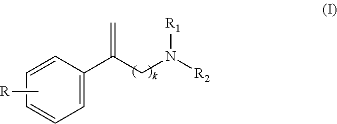

wherein k is an integer from 1 to 3, preferably 2; wherein R₁ is a methyl group or a phenyl group and R₂ is a benzyl group, or wherein R₁ and R₂ are connected to form a moiety containing one 6-membered ring, with four carbons, bearing an O or N—CH₃ group at the 4-position of the 6-membered ring; wherein R, in structure (I), is hydrogen, a phenyl ring co-attached at two neighboring ring carbon positions with the phenyl ring shown so as to form a naphthalene assembly, a phenyl group attached at a single carbon of the phenyl ring shown, a C₁-C₄ hydrocarbyl group (such as a methyl group), a C₁-C₆ hydrocarbyl group containing 1 to 4 additional heteroatoms (such as O, N, S, P, Se, and combinations thereof).

The inventive functional polymers disclosed herein based on functionalized styrenic monomers including a nitrogen-containing moiety, other than as pendant to the phenyl ring may be polymerized from the exemplary ADAMS monomers according to structure (I), which include, 1-benzylmethylamino-3-phenylbut-3-ene, 1-benzylphenylamino-3-phenylbut-3-ene, 1-(N-morpholinyl)-3-phenylbut-3-ene, and 1-(4-methyl-1-piperazinyl)-3-phenylbut-3-ene.

In some embodiments, the inventive functional polymers disclosed herein based on the functionalized styrenic monomers including a nitrogen-containing moiety, other than as pendant to the phenyl ring may be polymerized from the exemplary ADAMS monomers according to structure (I), may exhibit a k value of exactly 2.

For clarity, and as used herein, the inventive functional polymers disclosed herein based on the functionalized styrenic monomers including a nitrogen-containing moiety, other than as pendant to the phenyl ring may be polymerized from the exemplary ADAMS monomers of structure (I) having a vinylidene bond, such as originating from the olefinic double-bond in alpha-methylstyrene, which can be reflected in -3-ene/-3-enyl language of the IUPAC nomenclature, for example.

The polymer compositions disclosed herein may optionally contain residues of initiators and/or co-initiators that are, or may be, used in living or pseudo-living anionic polymerization reactions. Non-limiting examples may include alkyl residues from sec-butyllithium, n-butyllithium, tert-butyllithium, and the like, and combinations, reaction products, and/or degradation products thereof.

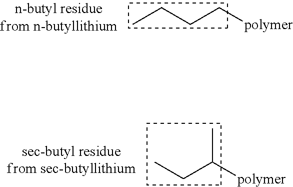

The alkyl residues from the initiators may optionally be present at one or more termini of the polymer backbone.

The initiator which may be used may be an alkyl lithium, alkyl sodium, or alkyl potassium compound, generally in the C2 to C12 range. Alkyl lithium compounds such as methyllithium, ethyllithium, n-propyllithium, isopropylithium, n-butyllithium, iso-butyllithium, sec-butyllithium, tert-butyllithium, n-amyllithium, iso-amyllithium, sec-amyllithium, tert-amyllithium, hexyllithium, or a combination thereof, are preferred. Secondary alkyl lithium compounds, such as sec-butyllithium, sec-amyllithium, or a combination thereof, are more preferred. Most preferred is sec-butyllithium. Substituted alkyllithiums may also be used, such as aralkyllithium compounds, for example, benzyllithium, 1-lithioethylbenzene, and 1-lithio-3-methylpentylbenzene.

Provided herein are inventive anionically polymerized polymers derived from functionalized styrenic monomers including a nitrogen-containing moiety, other than as pendant to the phenyl ring having the general structure:

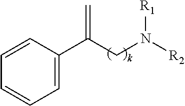

wherein R₁ is a methyl group or a phenyl group and R₂ is a benzyl group, or wherein R₁ and R₂ are connected to form a moiety containing one 6-membered ring, with four carbons, bearing an O or N—CH₃ group at the 4-position of the 6-membered ring; Monomers with k=2 are preferable due to ease of monomer synthesis and favorable reactivity in the polymerization. Alternatively, monomers with k>3 may be used, but are more complex to prepare and are less commercially feasible. Alternatively, monomers with k=1 may be used, but are difficult to polymerize via anionic polymerization.

The functionalized styrenic monomers including a nitrogen-containing moiety, other than as pendant to the phenyl ring, may be co-polymerized with isoprene.

Depending on the reactivity ratios of the styrenic monomers including a nitrogen-containing moiety, other than as pendant to the phenyl ring, and the isoprene present in the polymerization reaction, the repeat units of structure (V) may, in some cases, form alternating structures with the repeat units of structures (VIIₐ), (VII_{b}), or a combination thereof. For example, the reaction below, wherein R₁, R₂, and R₅, have the same meanings as indicated above.

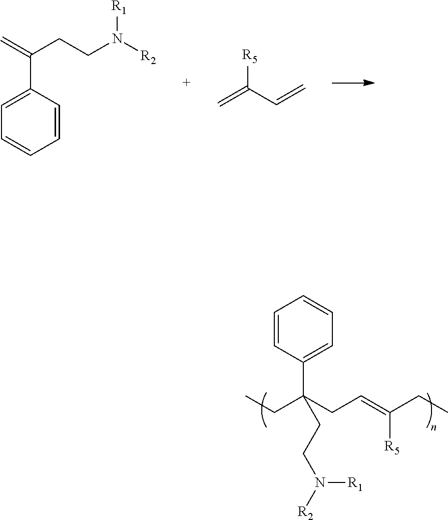

The alternating structures formed as a combination of repeat units of structure (V) with structures (VIIₐ), (VII_{b}), or a combination thereof, would thereby form larger repeat units of structures, (Xₐ), (X_{b}), or a combination thereof. With regard to the polymeric repeat unit of structures (VIIₐ) and (Xₐ), it the double bond may be in the cis-isomer form, the trans-isomer form, or combinations thereof.

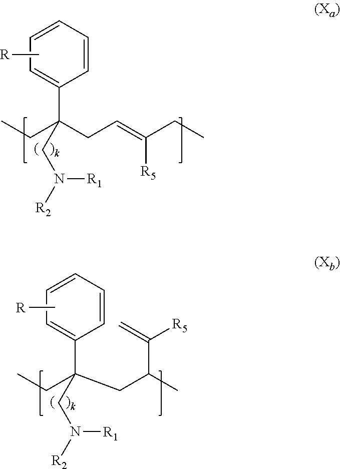

wherein k is an integer from 1 to 3, preferably 2; wherein R₁ is a methyl group or a phenyl group and R₂ is a benzyl group, or wherein R₁ and R₂ are connected to form a moiety containing one 6-membered ring, with four carbons, bearing an O or N—CH₃ group at the 4-position of the 6-membered ring; wherein R, in structure (I), is hydrogen, a phenyl ring co-attached at two neighboring ring carbon positions with the phenyl ring shown so as to form a naphthalene assembly, a phenyl group attached at a single carbon of the phenyl ring shown, a C₁-C₄ hydrocarbyl group (such as a methyl group), a C₁-C₆ hydrocarbyl group containing 1 to 4 additional heteroatoms (such as O, N, S, P, Se, and combinations thereof).

Additionally, depending on the reactivity ratios of the styrenic monomers including a nitrogen-containing moiety, other than as pendant to the phenyl ring, and the isoprene present in the polymerization reaction, and where a molar excess of isoprene is present in the polymerization reaction, the polymer may in some cases form a block of repeat units of structures (Xₐ), (X_{b}), or a combination thereof, followed by a second block of repeat units of structures (VIIₐ), (VII_{b}) or a combination thereof, absent of repeat units of structures (IX), (Xₐ), (X_{b}). For example, the reaction below, wherein R₁, R₂, and R₅ have the same meanings as indicated above. With regard to the polymeric repeat units, the double bonds may be in the cis-isomer form, the trans-isomer form, or combinations thereof.

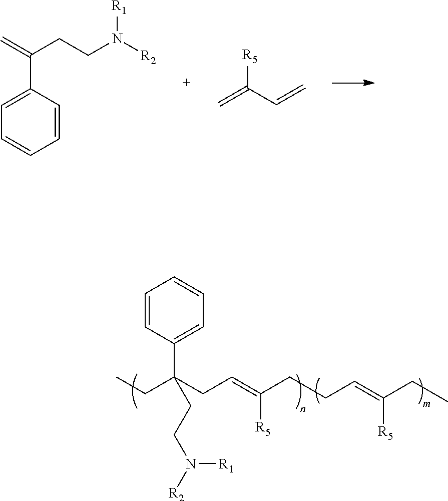

In some embodiments, additional portions of the monomers, or combinations thereof, may optionally be added to the polymerization reaction sequentially. In such cases, those monomers added later in the reaction may form one or more blocks of repeat units within the polymer with a different composition to those repeat units from monomers earlier in the polymerization.

In cases where the polymer contains two blocks of repeat units with different composition, either as a result of differences in the monomer reactivity ratios or from sequential addition of monomers to the polymerization reaction, the polymer is described as a ‘diblock’. Similarly, when the polymer contains three, four, five, or six blocks of repeat units of different composition, either as a result of differences in the monomer reactivity ratios or from sequential addition of monomers to the polymerization reaction, the polymers are described as ‘triblock’, ‘tetrablock’, ‘pentablock’, or ‘hexablock’, respectively.

In some embodiments, the polymer may be coupled using a polyfunctional coupling agent to form a polymer with a star architecture. Many suitable types of these polyfunctional compounds have been described in U.S. Pat. Nos. 3,595,941; 3,468,972, 3,135,716; 3,078,254, and 3,594,452, the disclosures of which are herein incorporated by reference in their entirety. The polyfunctional coupling agent may optionally be a halogen-substituted or alkoxy-substituted silane, including, tetrachlorosilane, tetramethoxysilane, tetraethoxysilane, bis-trimethoxy-silylethane, bis-triethoxy-silylethane, hexachlorodisiloxane, bis-trichlorosilylethane, 1,6-bis(trichlorosilyl)-hexane, or a combination thereof.

A preferred coupling agent is a polyalkenyl aromatic coupling agent. The most preferred coupling agent is divinyl benzene. Polyalkenyl aromatic coupling agents capable of forming star shaped polymers are known in the art. See generally, Canadian patent number 716,645 and U.S. Pat. Nos. 4,010,226 and 3,985,830 which are herein incorporated by reference in their entirety. A detailed description of a variety of such coupling agents is found in U.S. Pat. No. 4,391,949 which is herein incorporated by reference in its entirety. Examples of suitable polyvinyl aromatic compounds are 1,2-divinyl benzene, 1,3-divinylbenzene, 1,4-divinylbenzene, 1,2,4-trivinylbenzene, 1,3-divinylnaphthalene, 1,8-divinylnaphthalene, 1,3,5trivinylnaphthalene, 2,4-divinylbiphenyl, 3,5,4′-trivinylbiphenyl, 1,2-divinyl-3, 4-dimethylbenzene, 1,5,6-trivinyl-3,7-diethylnaphthalene, 1,3-divinyl-4, 5,6-tributyl naphthalene, 2,2′-divinyl-4-ethyl-4′-propylbiphenyl and the like, or a combination thereof.

In cases where a polyfunctional coupling agent is used to couple a polymer to form a polymer star architecture, the coupling ratio (CR) is used to refer to the amount of the polymer that has been crosslinked into a star architecture, meaning the percentage by weight of the polymer in a star architecture relative to the total weight of polymer in the sample. In some embodiments, the inventive functional polymers disclosed herein based on functionalized styrenic monomers including a nitrogen-containing moiety including a star polymer architecture may have a CR greater than 20%, or greater than 30%, or greater than 40%, or greater than 50%, or greater 60%, or greater than 70%, or greater than 80%, or greater than 90%, or greater than 95%.

In one embodiment, the inventive functional polymers disclosed herein based on functionalized styrene monomers including a nitrogen-containing moiety, other than as pendant to the phenyl ring, may be a hydrogenated copolymer of 1-benzylmethylamino-3-phenylbut-3-ene and isoprene, containing 10.0 to 20.0 wt % of repeat units corresponding to the reacted form of 1-benzylmethylamino-3-phenylbut-3-ene and having a peak-average molecular weight between 45.0 to 65.0 kDa.

In one embodiment, the inventive functional polymers disclosed herein based on functionalized styrene monomers including a nitrogen-containing moiety, other than as pendant to the phenyl ring, may be a hydrogenated copolymer of 1-(4-methyl-1-piperazinyl)-3-phenylbut-3-ene and isoprene, containing 5.0 to 10.0 wt % of repeat units corresponding to the reacted form of 1-(4-methyl-1-piperazinyl)-3-phenylbut-3-ene and having a peak-average molecular weight between 24.0 to 42.0 kDa.

In one embodiment, the inventive functional polymers disclosed herein based on functionalized styrene monomers including a nitrogen-containing moiety, other than as pendant to the phenyl ring, may be a hydrogenated copolymer of 1-(N-morpholinyl)-3-phenylbut-3-ene and isoprene, containing 4.0 to 5.0 wt % of repeat units corresponding to the reacted form of 1-(N-morpholinyl)-3-phenylbut-3-ene, and having a peak-average molecular weight between 36.0 to 46.0 kDa.

In one embodiment, the inventive functional polymers disclosed herein based on functionalized styrene monomers including a nitrogen-containing moiety, other than as pendant to the phenyl ring, may be a hydrogenated copolymer of 1-benzylphenylamino-3-phenylbut-3-ene and isoprene, containing 5.0 to 7.0 wt % of repeat units corresponding to the reacted form of 1-benzylphenylamino-3-phenylbut-3-ene and having a peak-average molecular weight between 140.0 to 180.0 kDa.

In some embodiments, the inventive functional polymers disclosed herein based on functionalized styrenic monomers including a nitrogen-containing moiety, other than as pendant to the phenyl ring, or based on functionalized conjugated (non-aromatic) monomers including a nitrogen-containing moiety, may optionally be further subjected to post-polymerization modification in order to modify their structure.

In some embodiments, the post-polymerization modification is hydrogenation. Hydrogenation can be carried out in the process of the present disclosure by known catalysis systems, including heterogeneous systems and soluble systems. Soluble systems are disclosed in U.S. Pat. No. 4,284,835 at column 1, line 65 through column 9, line 16, as well as U.S. Pat. No. 4,980,331 at column 3 line 40 through column 6, line 28, both of which are herein incorporated by reference.

The hydrogenated copolymers described above may be partially or substantially hydrogenated. In the context of the present disclosure, partially hydrogenated means that from 10% to 90%, or 20% to 90%, or 30% to 90%, or 40% to 90% of the non-aromatic double bonds have been saturated. Substantially hydrogenated means that greater than 90%, or greater than 92%, or greater than 94%, or greater than 96%, or greater than 98%, or greater than 99%, or greater than 99.5%, or greater than 99.9% of the non-aromatic bonds have been saturated.

Additional teachings to hydrogenation may be found in Rachapudy et al., Journal of Polymer Science: Polymer Physics Edition, Vol. 17, 1211-1222 (1979), which is incorporated herein by reference in its entirety. Table 1 of the article discloses several systems including palladium on various supports (calcium carbonate, but also barium sulfide). The Rachapudy et al. article discloses preparation of homogeneous catalysts and heterogeneous catalysts.

Additional teachings to hydrogenation processes and catalysts are disclosed in U.S. Pat. Nos. 4,284,835 and 4,980,331, both of which are incorporated herein by reference in their entirety.

In some embodiments, the post-polymerization modification may be a deprotection reaction which removes a cleavable chemical protecting group from the repeat units of structures (V), (VIII), or a combination thereof. A cleavable chemical protecting group means a chemical group which is inert under the polymerization reaction conditions, but can be removed with post-polymerization chemistry to yield a free —NH— or a free —NH2 functional group on the ADAMS repeat unit. In one such form, a preferred cleavable chemical protecting group is a benzyl group and the deprotection reaction is a hydrogenation reaction.

### Embodiment 1 of Inventive Copolymers and Use in Lubricants

In one form of the inventive copolymers disclosed herein, the copolymer may include: (a) 10.0 to 20.0 wt % of amine-derivatized alpha-methyl styrene (ADAMS) repeat units according to structure (I):

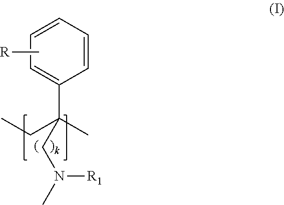

wherein: k is an integer from 1 to 3; R₁ a hydrogen or a benzyl group, R is hydrogen, a phenyl ring co-attached at two neighboring ring carbon positions with the phenyl ring shown so as to form a naphthalene assembly, a phenyl group attached at a single carbon of the phenyl ring shown, a C1-C4 hydrocarbyl group, a C1-C6 hydrocarbyl group containing 1 to 4 additional heteroatoms selected from the group consisting of O, N, S, P, Se, and combinations thereof, and (b) 80.0 to 90.0 wt % of repeat units corresponding to the reacted form of isoprene, and wherein the peak-average molecular weight of the copolymer is between 45.0 to 65.0 kDa. The copolymer may be partially or substantially hydrogenated. In a preferred form, the copolymer has k=2.

The copolymer may alternatively include an alkyl residue from a monofunctional initiator, including, but not limited to, alkyl lithium, alkyl sodium, alkyl potassium and combinations thereof, and present at one or more termini of a polymer backbone. The alkyl residues from the monofunctional initiator include, but are not limited to, methyl, ethyl, n-propyl, isopropyl, n-butyl, iso-butyl, sec-butyl, tert-butyl, n-amyl, iso-amyl, sec-amyl, tert-amyl, hexyl groups, or combinations thereof. The copolymer includes one or more blocks, wherein one or more polymer blocks of the copolymer form a distributed polymer architecture, a diblock, a triblock, a tetrablock, a pentablock, a hexablock, a star polymer architecture, or combinations thereof.

The copolymer of this embodiment is useful as an additive in a lubricating oil composition. More particularly, the copolymer of this embodiment may be used as a viscosity modifier, a friction modifier, a dispersant, an antiwear agent, or a combination thereof in a lubricating oil composition. Preferably, the copolymer of this embodiment may be used as a viscosity modifier in a lubricating oil composition. More particularly, a lubricating oil composition may include or result from the admixing of: (i) at least 50 wt % of one or more base oils, based upon the weight of the lubricating oil composition; (ii) one or more dispersants; (iii) one or more detergents; and (iv) one or more copolymers of this embodiment.

The lubricating oil composition including the copolymer of this embodiment may have an SAE viscosity grade of 20 W-X, 15 W-X, 10 W-X, 5 W-X, or 0 W-X, where X represents any one of 8, 12, 16, 20, 30, 40, or 50. Alternatively, the lubricating oil composition of this embodiment may include or result from the admixing of: (i) from 50 to 99 mass % of the one or more of the base oils, based upon the weight of the lubricating oil composition; (ii) from 0.01 to 20 wt % based on total weight of the lubricating oil composition, of the one or more dispersants; (iii) from 0.10 to 20 mass %, based upon the weight of the lubricating oil composition, of the one or more detergents; and (iv) from 0.10 to 20 mass %, based upon the weight of the lubricating oil composition, of the one or more copolymers. Alternatively, the lubricating oil composition of this embodiment may further include one, two, three, four, five, six or more of additional additives chosen from, but not limited to, friction modifiers; antioxidants; pour point depressants; anti-foam agents; viscosity modifiers; corrosion inhibitors and/or anti-rust agents; and antiwear agents.

Alternatively, the lubricating oil composition of this embodiment may further include one, two, three, four, five, six, or more of: A) from 0.01 to 5 wt %, based on total weight of the lubricating oil composition, of one or more friction modifiers; B) from 0.01 to 10 wt %, based on total weight of the lubricating oil composition, of one or more antioxidants; C) from 0.01 to 5 wt %, based on total weight of the lubricating oil composition, of one or more pour point depressants; D) from 0.001 to 5 wt %, based on total weight of the lubricating oil composition, of one or more anti-foam agents; E) from 0.001 to 10 wt %, based on total weight of the lubricating oil composition, of one or more viscosity modifiers; F) from 0.0 to 5 wt %, based on total weight of the lubricating oil composition, of one or more inhibitors and/or anti-rust agents; and/or G) from 0.001 to 10 wt %, based on total weight of the lubricating oil composition, of one or more antiwear agents. The one or more detergents of the composition may include one or more oil-soluble neutral or overbased sulfonates, phenates, sulfurized phenates, thiophosphonates, salicylates, naphthenates, and other oil-soluble carboxylates of an alkali or alkaline earth metal. The one or more dispersants of the composition may include one or more borated or unborated poly(alkenyl)succinimides, wherein the polyalkyenyl is derived from polyisobutylene and the imide is derived from a polyamine.

The lubricating oil composition including the copolymer of this embodiment may be used in a method of lubricating an internal combustion engine during operation of the engine including the steps of: (i) providing to a crankcase of the internal combustion engine the lubricating oil composition; (ii) providing a fuel in the internal combustion engine; and (iii) combusting the fuel in the internal combustion engine. Non-limiting exemplary fuels include one or more of hydrocarbon fuel, renewable fuel, hydrogen fuel, or any blend thereof. The lubricating oil composition of this embodiment may be used as a lubricating oil composition in internal combustion engines, including, but not limited to, natural gas engines, gasoline engines, diesel engines and stationary engines.

### Embodiment 2 of Inventive Copolymers and Use in Lubricants

In another form of the inventive copolymers disclosed herein, the copolymer may include: (a) 5.0 to 10.0 wt % of amine-derivatized alpha-methyl styrene (ADAMS) repeat units according to structure (II):

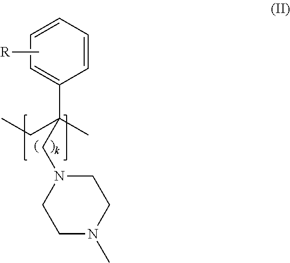

wherein: k is an integer from 1 to 3; R is hydrogen, a phenyl ring co-attached at two neighboring ring carbon positions with the phenyl ring shown so as to form a naphthalene assembly, a phenyl group attached at a single carbon of the phenyl ring shown, a C1-C4 hydrocarbyl group, a C1-C6 hydrocarbyl group containing 1 to 4 additional heteroatoms selected from the group consisting of O, N, S, P, Se, and combinations thereof, and (b) the remaining 90.0 to 95.0 wt % of repeat units corresponding to the reacted form of isoprene, and wherein, the peak-average molecular weight of the copolymer is between 24.0 to 42.0 kDa. The copolymer may be partially or substantially hydrogenated. In a preferred form, the copolymer has k=2.

The copolymer may alternatively include an alkyl residue from a monofunctional initiator, including, but not limited to, alkyl lithium, alkyl sodium, alkyl potassium and combinations thereof, and present at one or more termini of a polymer backbone. The alkyl residues from the monofunctional initiator include, but are not limited to, methyl, ethyl, n-propyl, isopropyl, n-butyl, iso-butyl, sec-butyl, tert-butyl, n-amyl, iso-amyl, sec-amyl, tert-amyl, hexyl groups, or combinations thereof. The copolymer includes one or more blocks, wherein one or more polymer blocks of the copolymer form a distributed polymer architecture, a diblock, a triblock, a tetrablock, a pentablock, a hexablock, a star polymer architecture, or combinations thereof.

The copolymer of this embodiment is also useful as an additive in a lubricating oil composition. More particularly, the copolymer of this embodiment may be used as a viscosity modifier, a friction modifier, a dispersant, an antiwear agent, or a combination thereof in a lubricating oil composition. Preferably, the copolymer of this embodiment may be used as a viscosity modifier in a lubricating oil composition. More particularly, a lubricating oil composition may include or result from the admixing of: (i) at least 50 wt % of one or more base oils, based upon the weight of the lubricating oil composition; (ii) one or more dispersants; (iii) one or more detergents; and (iv) one or more copolymers of this embodiment.

The lubricating oil composition including the copolymer of this embodiment may have an SAE viscosity grade of 20 W-X, 15 W-X, 10 W-X, 5 W-X, or OW-X, where X represents any one of 8, 12, 16, 20, 30, 40, or 50. Alternatively, the lubricating oil composition of this embodiment may include or result from the admixing of: (i) from 50 to 99 mass % of the one or more of the base oils, based upon the weight of the lubricating oil composition; (ii) from 0.01 to 20 wt % based on total weight of the lubricating oil composition, of the one or more dispersants; (iii) from 0.10 to 20 mass %, based upon the weight of the lubricating oil composition, of the one or more detergents; and (iv) from 0.10 to 20 mass %, based upon the weight of the lubricating oil composition, of the one or more copolymers. Alternatively, the lubricating oil composition of this embodiment may further include one, two, three, four, five, six or more of additional additives chosen from, but not limited to, friction modifiers; antioxidants; pour point depressants; anti-foam agents; viscosity modifiers; corrosion inhibitors and/or anti-rust agents; and antiwear agents.

Alternatively, the lubricating oil composition of this embodiment may further include one, two, three, four, five, six, or more of: A) from 0.01 to 5 wt %, based on total weight of the lubricating oil composition, of one or more friction modifiers; B) from 0.01 to 10 wt %, based on total weight of the lubricating oil composition, of one or more antioxidants; C) from 0.01 to 5 wt %, based on total weight of the lubricating oil composition, of one or more pour point depressants; D) from 0.001 to 5 wt %, based on total weight of the lubricating oil composition, of one or more anti-foam agents; E) from 0.001 to 10 wt %, based on total weight of the lubricating oil composition, of one or more viscosity modifiers; F) from 0.0 to 5 wt %, based on total weight of the lubricating oil composition, of one or more inhibitors and/or anti-rust agents; and/or G) from 0.001 to 10 wt %, based on total weight of the lubricating oil composition, of one or more antiwear agents. The one or more detergents of the composition may include one or more oil-soluble neutral or overbased sulfonates, phenates, sulfurized phenates, thiophosphonates, salicylates, naphthenates, and other oil-soluble carboxylates of an alkali or alkaline earth metal. The one or more dispersants of the composition may include one or more borated or unborated poly(alkenyl)succinimides, wherein the polyalkyenyl is derived from polyisobutylene and the imide is derived from a polyamine.

The lubricating oil composition including the copolymer of this embodiment may be used in a method of lubricating an internal combustion engine during operation of the engine including the steps of: (i) providing to a crankcase of the internal combustion engine the lubricating oil composition; (ii) providing a fuel in the internal combustion engine; and (iii) combusting the fuel in the internal combustion engine. Non-limiting exemplary fuels include one or more of hydrocarbon fuel, renewable fuel, hydrogen fuel, or any blend thereof. The lubricating oil composition of this embodiment may be used as a lubricating oil composition in internal combustion engines, including, but not limited to, natural gas engines, gasoline engines, diesel engines and stationary engines.

### Embodiment 3 of Inventive Copolymers and Use in Lubricants

In yet another form of the inventive copolymers disclosed herein, the copolymer may include: (a) 4.0 to 6.0 wt % of amine-derivatized alpha-methyl styrene (ADAMS) repeat units according to structure (III):

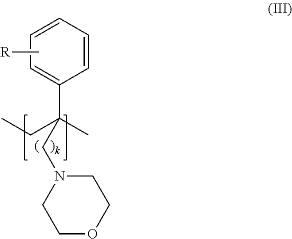

wherein: k is an integer from 1 to 3; R is hydrogen, a phenyl ring co-attached at two neighboring ring carbon positions with the phenyl ring shown so as to form a naphthalene assembly, a phenyl group attached at a single carbon of the phenyl ring shown, a C1-C4 hydrocarbyl group, a C1-C6 hydrocarbyl group containing 1 to 4 additional heteroatoms selected from the group consisting of O, N, S, P, Se, and combinations thereof, (b) the remaining 94.0 to 96.0 wt % of repeat units corresponding to the reacted form of isoprene, and wherein, the peak-average molecular weight of the copolymer is between 36.0 to 46.0 kDa. The copolymer may be partially or substantially hydrogenated. In a preferred form, the copolymer has k=2.

The copolymer may alternatively include an alkyl residue from a monofunctional initiator, including, but not limited to, alkyl lithium, alkyl sodium, alkyl potassium and combinations thereof, and present at one or more termini of a polymer backbone. The alkyl residues from the monofunctional initiator include, but are not limited to, methyl, ethyl, n-propyl, isopropyl, n-butyl, iso-butyl, sec-butyl, tert-butyl, n-amyl, iso-amyl, sec-amyl, tert-amyl, hexyl groups, or combinations thereof. The copolymer includes one or more blocks, wherein one or more polymer blocks of the copolymer form a distributed polymer architecture, a diblock, a triblock, a tetrablock, a pentablock, a hexablock, a star polymer architecture, or combinations thereof.

The copolymer of this embodiment is also useful as an additive in a lubricating oil composition. More particularly, the copolymer of this embodiment may be used as a viscosity modifier, a friction modifier, a dispersant, an antiwear agent, or a combination thereof in a lubricating oil composition. Preferably, the copolymer of this embodiment may be used as a viscosity modifier in a lubricating oil composition. More particularly, a lubricating oil composition may include or result from the admixing of: (i) at least 50 wt % of one or more base oils, based upon the weight of the lubricating oil composition; (ii) one or more dispersants; (iii) one or more detergents; and (iv) one or more copolymers of this embodiment.

The lubricating oil composition including the copolymer of this embodiment may have an SAE viscosity grade of 20 W-X, 15 W-X, 10 W-X, 5 W-X, or 0 W-X, where X represents any one of 8, 12, 16, 20, 30, 40, or 50. Alternatively, the lubricating oil composition of this embodiment may include or result from the admixing of: (i) from 50 to 99 mass % of the one or more of the base oils, based upon the weight of the lubricating oil composition; (ii) from 0.01 to 20 wt % based on total weight of the lubricating oil composition, of the one or more dispersants; (iii) from 0.10 to 20 mass %, based upon the weight of the lubricating oil composition, of the one or more detergents; and (iv) from 0.10 to 20 mass %, based upon the weight of the lubricating oil composition, of the one or more copolymers. Alternatively, the lubricating oil composition of this embodiment may further include one, two, three, four, five, six or more of additional additives chosen from, but not limited to, friction modifiers; antioxidants; pour point depressants; anti-foam agents; viscosity modifiers; corrosion inhibitors and/or anti-rust agents; and antiwear agents.

Alternatively, the lubricating oil composition of this embodiment may further include one, two, three, four, five, six, or more of: A) from 0.01 to 5 wt %, based on total weight of the lubricating oil composition, of one or more friction modifiers; B) from 0.01 to 10 wt %, based on total weight of the lubricating oil composition, of one or more antioxidants; C) from 0.01 to 5 wt %, based on total weight of the lubricating oil composition, of one or more pour point depressants; D) from 0.001 to 5 wt %, based on total weight of the lubricating oil composition, of one or more anti-foam agents; E) from 0.001 to 10 wt %, based on total weight of the lubricating oil composition, of one or more viscosity modifiers; F) from 0.0 to 5 wt %, based on total weight of the lubricating oil composition, of one or more inhibitors and/or anti-rust agents; and/or G) from 0.001 to 10 wt %, based on total weight of the lubricating oil composition, of one or more antiwear agents. The one or more detergents of the composition may include one or more oil-soluble neutral or overbased sulfonates, phenates, sulfurized phenates, thiophosphonates, salicylates, naphthenates, and other oil-soluble carboxylates of an alkali or alkaline earth metal. The one or more dispersants of the composition may include one or more borated or unborated poly(alkenyl)succinimides, wherein the polyalkyenyl is derived from polyisobutylene and the imide is derived from a polyamine.

The lubricating oil composition including the copolymer of this embodiment may be used in a method of lubricating an internal combustion engine during operation of the engine including the steps of: (i) providing to a crankcase of the internal combustion engine the lubricating oil composition; (ii) providing a fuel in the internal combustion engine; and (iii) combusting the fuel in the internal combustion engine. Non-limiting exemplary fuels include one or more of hydrocarbon fuel, renewable fuel, hydrogen fuel, or any blend thereof. The lubricating oil composition of this embodiment may be used as a lubricating oil composition in internal combustion engines, including, but not limited to, natural gas engines, gasoline engines, diesel engines and stationary engines.

### Embodiment 4 of Inventive Copolymers and Use in Lubricants

In still yet another form of the inventive copolymers disclosed herein, the copolymer may include (a) one or more amine-derivatized alpha-methyl styrene (ADAMS) repeat units according to structure (IV):

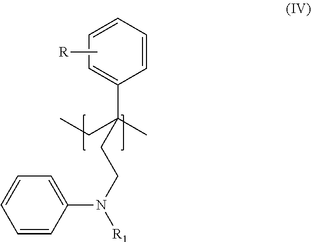

wherein: k is an integer from 1 to 3; R₁ a hydrogen or a benzyl group, R is hydrogen, a phenyl ring co-attached at two neighboring ring carbon positions with the phenyl ring shown so as to form a naphthalene assembly, a phenyl group attached at a single carbon of the phenyl ring shown, a C1-C4 hydrocarbyl group, a C1-C6 hydrocarbyl group containing 1 to 4 additional heteroatoms selected from the group consisting of O, N, S, P, Se, and combinations thereof, and (b) the remaining repeat units corresponding to the reacted form of isoprene. The copolymer may be partially or substantially hydrogenated. In a preferred form, the copolymer has k=2.

The copolymer may alternatively include an alkyl residue from a monofunctional initiator, including, but not limited to, alkyl lithium, alkyl sodium, alkyl potassium and combinations thereof, and present at one or more termini of a polymer backbone. The alkyl residues from the monofunctional initiator include, but are not limited to, methyl, ethyl, n-propyl, isopropyl, n-butyl, iso-butyl, sec-butyl, tert-butyl, n-amyl, iso-amyl, sec-amyl, tert-amyl, hexyl groups, or combinations thereof. The copolymer includes one or more blocks, wherein one or more polymer blocks of the copolymer form a distributed polymer architecture, a diblock, a triblock, a tetrablock, a pentablock, a hexablock, a star polymer architecture, or combinations thereof.

The copolymer of this embodiment is also useful as an additive in a lubricating oil composition. More particularly, the copolymer of this embodiment may be used as a viscosity modifier, a friction modifier, a dispersant, an antiwear agent, or a combination thereof in a lubricating oil composition. Preferably, the copolymer of this embodiment may be used as a viscosity modifier in a lubricating oil composition. More particularly, a lubricating oil composition may include or result from the admixing of: (i) at least 50 wt % of one or more base oils, based upon the weight of the lubricating oil composition; (ii) one or more dispersants; (iii) one or more detergents; and (iv) one or more copolymers of this embodiment.

The lubricating oil composition including the copolymer of this embodiment may have an SAE viscosity grade of 20 W-X, 15 W-X, 10 W-X, 5 W-X, or OW-X, where X represents any one of 8, 12, 16, 20, 30, 40, or 50. Alternatively, the lubricating oil composition of this embodiment may include or result from the admixing of: (i) from 50 to 99 mass % of the one or more of the base oils, based upon the weight of the lubricating oil composition; (ii) from 0.01 to 20 wt % based on total weight of the lubricating oil composition, of the one or more dispersants; (iii) from 0.10 to 20 mass %, based upon the weight of the lubricating oil composition, of the one or more detergents; and (iv) from 0.10 to 20 mass %, based upon the weight of the lubricating oil composition, of the one or more copolymers. Alternatively, the lubricating oil composition of this embodiment may further include one, two, three, four, five, six or more of additional additives chosen from, but not limited to, friction modifiers; antioxidants; pour point depressants; anti-foam agents; viscosity modifiers; corrosion inhibitors and/or anti-rust agents; and antiwear agents.

Alternatively, the lubricating oil composition of this embodiment may further include one, two, three, four, five, six, or more of: A) from 0.01 to 5 wt %, based on total weight of the lubricating oil composition, of one or more friction modifiers; B) from 0.01 to 10 wt %, based on total weight of the lubricating oil composition, of one or more antioxidants; C) from 0.01 to 5 wt %, based on total weight of the lubricating oil composition, of one or more pour point depressants; D) from 0.001 to 5 wt %, based on total weight of the lubricating oil composition, of one or more anti-foam agents; E) from 0.001 to 10 wt %, based on total weight of the lubricating oil composition, of one or more viscosity modifiers; F) from 0.0 to 5 wt %, based on total weight of the lubricating oil composition, of one or more inhibitors and/or anti-rust agents; and/or G) from 0.001 to 10 wt %, based on total weight of the lubricating oil composition, of one or more antiwear agents. The one or more detergents of the composition may include one or more oil-soluble neutral or overbased sulfonates, phenates, sulfurized phenates, thiophosphonates, salicylates, naphthenates, and other oil-soluble carboxylates of an alkali or alkaline earth metal. The one or more dispersants of the composition may include one or more borated or unborated poly(alkenyl)succinimides, wherein the polyalkyenyl is derived from polyisobutylene and the imide is derived from a polyamine.

The lubricating oil composition including the copolymer of this embodiment may be used in a method of lubricating an internal combustion engine during operation of the engine including the steps of: (i) providing to a crankcase of the internal combustion engine the lubricating oil composition; (ii) providing a fuel in the internal combustion engine; and (iii) combusting the fuel in the internal combustion engine. Non-limiting exemplary fuels include one or more of hydrocarbon fuel, renewable fuel, hydrogen fuel, or any blend thereof. The lubricating oil composition of this embodiment may be used as a lubricating oil composition in internal combustion engines, including, but not limited to, natural gas engines, gasoline engines, diesel engines and stationary engines.

### Methods of Making the ADAMS Copolymers

The novel polymers of functionalized styrenic monomers including a nitrogen-containing moiety, other than as pendant to the phenyl ring may be made by anionic polymerization processes.

Anionic polymerization processes which are absent of functionalized styrenic monomers including a nitrogen-containing moiety, other than as pendant to the phenyl ring are generally known in the art, and are described for example in U.S. Pat. Nos. 5,736,612, 5,773,521, 8,604,136, and 9,809,671, which are herein incorporated by reference in their entirety. Anionic polymerization processes generally comprise at least the following steps:
(a) polymerizing one or more monomers in an inert hydrocarbon solvent in the presence of an alkyl lithium initiator until substantially complete conversion;
(b) optionally adding one or more sequential additions of one or more monomers, of the same or different composition, allowing each sequential addition of said monomers to polymerize until substantially complete conversion;
(c) optionally adding a polyfunctional coupling agent to couple some or all of the polymer or copolymer;
(d) adding a terminating agent.

Anionic polymerizations are generally initiated with alkyl lithium reagents, most frequently with sec-butyllithium, although other mono- and di-functional alkyl lithium initiators can be used. [Lintsell, et al., Synthesis and characterization of α, ω- and α-functionalized hydrogenated polybutadienes: telechelic and semi-telechelic amine and phosophite terminated polymers, Polymer, Vol. 38, Number 11, 2835 (1997)].

The monofunctional initiator which may be used may be an alkyl lithium, alkyl sodium, or alkyl potassium compound, generally in the C2 to C12 range. Alkyl lithium compounds such as methyllithium, ethyllithium, n-propyllithium, isopropylithium, n-butyllithium, iso-butyllithium, sec-butyllithium, tert-butyllithium, n-amyllithium, iso-amyllithium, sec-amyllithium, tert-amyllithium, hexyllithium, or a combination thereof, are preferred. Secondary alkyl lithium compounds, such as sec-butyllithium, sec-amyllithium, or a combination thereof, are more preferred. Most preferred is sec-butyllithium. Substituted alkyllithiums may also be used, such as aralkyllithium compounds, for example, benzyllithium, 1-lithioethylbenzene, and 1-lithio-3-methylpentylbenzene.

The functionalized styrenic monomers including a nitrogen-containing moiety, other than as pendant to the phenyl ring, is co-polymerized with isoprene.

The novel polymers of functionalized styrenic monomers including a nitrogen-containing moiety, other than as pendant to the phenyl ring, may be prepared via anionic polymerization process in which the monomers, or combinations thereof, are polymerized in solution in an inert hydrocarbon solvent in the presence of an alkyl lithium initiator. The inert hydrocarbon solvent may be any hydrocarbon, generally from 5 to 8 carbons, or mixtures thereof, which does not react with the alkyl lithium initiator or the ‘living’ anionic chain end of the polymer backbone, and offers appropriate solubility characteristics for the product polymer. Non-limiting examples of appropriate solvents are cyclic alkanes, such as cyclopentane, cyclohexane, cycloheptane, and cyclooctane, all of which are relatively non-polar. Other suitable solvents will be known to those skilled in the art and can be selected to perform effectively in a given set of process conditions, with polymerization temperature being one of the major factors taken into consideration.

The polymerization is preferably conducted in the presence of a polar additive that reduces the associations between the ions at the reactive ‘living’ anionic chain end of the polymer backbone, and thereby promotes polymerization. Non-limiting examples of the polar additives may include various ethers (i.e., dimethyl ether, diethyl ether, tetrahydrofuran, 2-methyl tetrahydrofuran, anisole 1,2-dimethoxyethane, 1,2-diethoxyethane, 1,2-dimethoxybenzene, 1-methoxy-2-(2-methoxyethoxy)ethane, and the like), various amines (i.e., trimethylamine, triethylamine, N,N,N′,N′-tetramethyl ethylene diamine, N,N,N′,N″,N″-pentamethyl diethylene triamine, and the like), or combinations thereof. Of the above polar additives, ethers are preferred. More preferred are diethyl ether, tetrahydrofuran, 1,2-dimethoxyethane, or a combination thereof.

Polymerization reaction conditions to prepare the novel polymers of functionalized styrenic monomers including a nitrogen-containing moiety, other than as pendant to the phenyl ring, are typically similar to those used for anionic polymerizations in general. Depending on the monomers and the reaction solvent, the polymerization reaction may be carried out at a temperature of from about −80° C. to about 200° C., alternatively from about −40° C. to about 150° C., preferably from about 0° C. to about 100° C., and more preferably, from about 20° C. to about 90° C. In some examples, the polymerization of the functionalized monomers and copolymerization with other monomers and blocks can be carried out at room temperature, or alternatively from 15 to 70° C., alternatively from 20 to 60° C., alternatively from 25 to 50° C., or combinations of these aforementioned temperatures, or individual temperatures within such ranges.

The polymerization reaction is carried out under a dry, inert atmosphere, preferably nitrogen, and may also be carried out under pressure within the range of from about 0 bar to about 10 bar.

Upon completion of the polymerization reaction, a terminating agent may be added to stop the reaction, and quench the reactive ‘living’ anionic chain end of the polymer backbone. The polymerization terminating agent can be either various primary or secondary alcohols or an epoxide terminating agent. Non-limiting examples of the various primary or secondary alcohols include methanol, ethanol, isopropanol, 2-ethyl-1-hexanol, and the like, or a combination thereof. Non-limiting examples of the epoxide terminating agent include ethylene oxide, propylene oxide, butylene oxide, styrene oxide, methyl glycidyl ether, ethyl glycidyl ether, propyl glycidyl ether, butyl glycidyl ether, benzyl glycidyl ether, phenyl glycidyl ether, and the like, or a combination thereof. Of the polymerization terminating agents, methanol or isopropanol are preferred, except where one or more —OH functional groups at one or more termini of the polymer chain are desired, in which case, ethylene oxide or propylene oxide are preferred.

The novel polymers of functionalized styrenic monomers including a nitrogen-containing moiety, other than as pendant to the phenyl ring, may optionally be isolated or purified according to various general polymer isolation or purification techniques which are known in the art, for example, pouring the polymerization reaction solution into a poor solvent of the polymer, such as methanol, to solidify the polymers, or pouring the polymerization reaction solution into hot water together with steam to remove the solvent by azeotropy (steam stripping) and drying the resultant product.

The inventive monomers disclosed in U.S. Provisional Application Ser. No. 63/483,365 have, to the best of the Applicant's knowledge, not been previously polymerized.

### Concentrates

A concentrate, also referred to as an additive package, adpak, or addpack, is a composition having less than 50 mass % (such as less than 40 mass %, such as less than 30 mass %, such as less than 25 mass %, such as less than 20 mass %) base oil and lubricant composition additives (such as described herein) which is typically then further blended with additional base oil to form a lubricating oil product.

This disclosure relates to concentrate compositions comprising or resulting from the admixing of:
(a) from 1 to less than 50 mass % (alternately 5 to 45 mass %, alternately 7 to 40 mass %, alternately 10 to 35 mass %, alternately 10 to 25 mass %) of one or more base oils, based upon the weight of the lubricating composition;
(b) from 0.10 to 20 mass % (in particular 0.2 to 15 mass %, alternately 0.5 mass % to 10 mass %, alternately 1 to 7 mass %), based upon the weight of the composition, of one or more copolymers selected the following and comprising:
   I. (a) 10.0 to 20.0 wt % of amine-derivatized alpha-methyl styrene (ADAMS) repeat units according to structure (I):

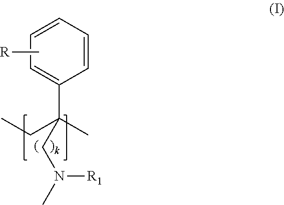

wherein: k is an integer from 1 to 3; R₁ a hydrogen or a benzyl group, R is hydrogen, a phenyl ring co-attached at two neighboring ring carbon positions with the phenyl ring shown so as to form a naphthalene assembly, a phenyl group attached at a single carbon of the phenyl ring shown, a C₁-C₄ hydrocarbyl group, a C₁-C₆ hydrocarbyl group containing 1 to 4 additional heteroatoms selected from the group consisting of O, N, S, P, Se, and combinations thereof, and (b) 80.0 to 90.0 wt % of repeat units corresponding to the reacted form of isoprene, and wherein the peak-average molecular weight of the copolymer is between 45.0 to 65.0 kDa;
II. (a) 5.0 to 10.0 wt % of amine-derivatized alpha-methyl styrene (ADAMS) repeat units according to structure (II):

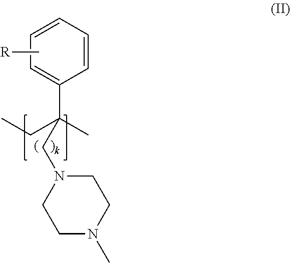

wherein: k is an integer from 1 to 3; R is hydrogen, a phenyl ring co-attached at two neighboring ring carbon positions with the phenyl ring shown so as to form a naphthalene assembly, a phenyl group attached at a single carbon of the phenyl ring shown, a C1-C4 hydrocarbyl group, a C1-C6 hydrocarbyl group containing 1 to 4 additional heteroatoms selected from the group consisting of O, N, S, P, Se, and combinations thereof, and (b) the remaining 90.0 to 95.0 wt % of repeat units corresponding to the reacted form of isoprene, and wherein, the peak-average molecular weight of the copolymer is between 24.0 to 42.0 kDa;
III. (a) 4.0 to 6.0 wt % of amine-derivatized alpha-methyl styrene (ADAMS) repeat units according to structure (III):

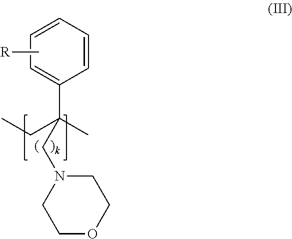

wherein: k is an integer from 1 to 3; R is hydrogen, a phenyl ring co-attached at two neighboring ring carbon positions with the phenyl ring shown so as to form a naphthalene assembly, a phenyl group attached at a single carbon of the phenyl ring shown, a C1-C4 hydrocarbyl group, a C1-C6 hydrocarbyl group containing 1 to 4 additional heteroatoms selected from the group consisting of O, N, S, P, Se, and combinations thereof, and (b) the remaining 94.0 to 96.0 wt % of repeat units corresponding to the reacted form of isoprene, and wherein, the peak-average molecular weight of the copolymer is between 36.0 to 46.0 kDa;
IV. (a) one or more amine-derivatized alpha-methyl styrene (ADAMS) repeat units according to structure (IV):

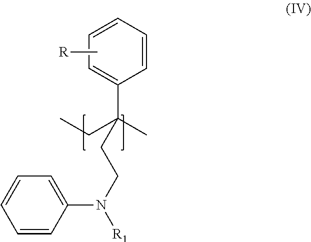

wherein: k is an integer from 1 to 3; R₁ a hydrogen or a benzyl group, R is hydrogen, a phenyl ring co-attached at two neighboring ring carbon positions with the phenyl ring shown so as to form a naphthalene assembly, a phenyl group attached at a single carbon of the phenyl ring shown, a C₁-C₄ hydrocarbyl group, a C₁-C₆ hydrocarbyl group containing 1 to 4 additional heteroatoms selected from the group consisting of O, N, S, P, Se, and combinations thereof, and (b) the remaining repeat units corresponding to the reacted form of isoprene.

This disclosure relates to concentrate compositions comprising or resulting from the admixing of:
(i) from 1 to less than 50 mass % (alternately 5 to 45 mass %, alternately 7 to 40 mass %, alternately 10 to 35 mass %, alternately 10 to 25 mass %), based upon the weight of the composition, of one or more base oil(s);
(ii) from 0.10 to 20 mass % (in particular 0.15 to 10 mass %, alternately 0.20 mass % to 5 mass %, alternately 0.25 to 2 mass %), based upon the weight of the composition, of one or more detergent(s);
(iii) from 0.10 to 20 mass % (in particular 0.15 to 10 mass %, alternately 0.20 mass % to 5 mass %, alternately 0.25 to 2 mass %), based upon the weight of the composition, of one or more dispersant(s) (such as PIBSA-PAM); and
(iv) from 0.10 to 20 mass % (in particular 0.15 to 10 mass %, alternately 0.20 mass % to 5 mass %, alternately 0.25 to 2 mass %), based upon the weight of the composition, of one or more inventive copolymers described herein;
(v) optional additional components, antioxidants, pour point depressants, anti-foam agents, viscosity modifiers, corrosion inhibitors, antiwear agents, extreme pressure additives, demulsifiers, seal compatibility agents, additive diluent base oils, friction modifier(s) (such as, organic FM, such as organic ester, such as fatty acid ester), etc.

In embodiments, the concentrate composition may optionally be absent solvent (such as aliphatic or aromatic solvent) and/or absent functionalized base oil.

This disclosure also relates to concentrate compositions comprising or resulting from the admixing of:
A) from 1 to less than 50 mass % (alternately 5 to 45 mass %, alternately 7 to 40 mass %, alternately 10 to 35 mass %, alternately 10 to 25 mass %), based upon the weight of the concentrate composition, of one or more base oil(s);
B) from 0.10 to 20 mass % (in particular, 0.15 to 10 mass %, alternately 0.20 mass % to 5 mass %, alternately 0.25 to 3 mass %), based upon the weight of the concentrate composition, of one or more inventive copolymers described herein;
C) from 0.1 to 20 wt % (in particular 0.5 to 10 mass %, alternately 2 to 6 mass %), based on total weight of the concentrate composition, of one or more detergent(s) (such as blends of detergents);
D) optionally, from 0.01 to 5 wt % (in particular, 0.1 to 4 mass %, alternately 0.25 to 3 mass %, alternately 0.25 to 0.075 mass %), based on total weight of the concentrate composition, of one or more friction modifier(s) (such as organic friction modifiers, such as glycerol monoeoleate);
E) optionally, from 0.01 to 20 wt % (in particular, 0.01 to 15 mass %, alternately 0.1 to 10 mass %), based on total weight of the concentrate composition, of one or more antioxidant(s) (such as blends of antioxidants);
F) optionally, from 0.01 to 5 wt % (in particular, 0.01 to 3 mass %, alternately 0.1 to 1.5 mass %), based on total weight of the concentrate composition, of one or more pour point depressants (such as blends of pour point depressants);
G) optionally, from 0.001 to 5 wt % (in particular, 0.01 to 3 mass %, alternately 0.02 to 1 mass %), based on total weight of the concentrate composition, of one or more anti-foam agents (such as blends of anti-foam agents);
I) optionally, from 0.01 to 40 wt % (in particular, 0.1 to 30 mass %, alternately 1 to 20 mass %), based on total weight of the concentrate composition, of one or more dispersants (such as blends of dispersants);
K) optionally, from 0.001 to 10 wt % (in particular, 0.1 to 8 mass %, alternately 1 to 5 mass % alternately 0.25 to 0.075 mass %), based on total weight of the lubricating composition, of one or more antiwear agents (such as blends of antiwear agents, such as ZDDP).

Optionally, the concentrate may be absent functionalized oil.

In embodiments, the concentrate composition may optionally be absent solvent (such as aliphatic or aromatic solvent) and/or absent functionalized base oil.

Optionally, the concentrate may be absent phenolic antioxidant.

In embodiments, the concentrate may comprise less than 75 ppm boron, alternately less than 60 ppm boron, alternately from 1 to 70 ppm boron. Alternately, the concentrate may be absent boron.

In embodiments, the concentrate may comprise less than 20 (such as 15, such as 10, such as 5, such as 3, such as 1) mass %, functionalized (such as aminated) polybutene (such as polyisobutylene), such as PIBSA-PAM. In embodiments, the concentrate comprises is substantially free or absent, functionalized (such as aminated) polybutene (such as polyisobutylene), such as PIBSA-PAM.

In embodiments, the concentrate may comprise acylated polymers, such as polyisobutylene succinic acid, optionally, having an Mn of 500 to 50,000 g/mol, such as 600 to 5,000 g/mol, such as 700 to 3000 g/mol. In embodiments, the concentrate may comprise acylated polymers, such as polyisobutylene succinic acid, having an Mn of 500 1600 g/mol, such as 700 to 1200 g/mol.

In embodiments, the concentrate may comprise 20 (such as 15, such as 10, such as 5, such as 3, such as 1) mass % or less block copolymer, such as block, star, random, and/or tapered block copolymer.

In embodiments, the concentrate may be substantially free of or absent block copolymer, such as block, star, random, and/or tapered block copolymer.

In embodiments, the concentrate may comprise 20 mass % or less (such as 15 mass % or less, such as 10 mass % or less, such as 5 mass % or less, such as 3 mass % or less, such as 1) mass % or less styrenic copolymer, such as block, star, random, and/or tapered styrenic block copolymer).

In embodiments, the concentrate may be substantially free of or absent styrenic copolymer, such as block, star, random, and/or tapered sytrenic block copolymer).

In embodiments, the concentrate may comprise less than 20 (such as less than 15, such as 10, such as less than 5, such as less than 3, such as 1) mass % of functionalized diluent, such as functionalized oil.

In embodiments, the concentrate may substantially free of or absent functionalized diluent, such as functionalized oil.

In embodiments, the concentrate may comprise less than 0.5 (such as less than 0.4, such as less than 0.3, such as less than 0.2, such as 0.1, substantially absent, no) wt %, based upon the weight of the concentrate, of secondary hydrocarbyl amine compounds and tertiary hydrocarbyl amine compounds.

In embodiments, the concentrate may be substantially absent, or may comprise no, secondary hydrocarbyl amine compounds and tertiary hydrocarbyl amine compounds.

In embodiments, the concentrate may have a kinematic viscosity at 100° C. of less than 1000 cSt, such as less than 500 cSt, such as less than 200 cSt.

This disclosure also relates to methods of making concentrate compositions comprising combining: (a) from 1 to less than 50 mass % (alternately 5 to 45 mass %, alternately 7 to 40 mass %, alternately 10 to 35 mass %, alternately 10 to 25 mass %) of one or more base oils, based upon the weight of the lubricating composition; and (b) from 0.10 to 20 mass % (in particular 0.2 to 15 mass %, alternately 0.5 mass % to 10 mass %, alternately 1 to 7 mass %), based upon the weight of the composition, of one or more inventive copolymers described herein.

### Lubricating Oil Composition Components and Concentrate Components

### A. Base Oil

The base oil (also referred to as “base stock,” “lubricating oil basestock,” or “oil of lubricating viscosity”) useful herein may be a single oil or a blend of oils, and is typically a large liquid constituent of a lubricating composition, also referred to as a lubricant, into which additives and optional additional oils are blended, for example, to produce a lubricating composition, such as a final lubricant composition, a concentrate, or other lubricating composition.

A base oil may be selected from vegetable, animal, mineral, and synthetic lubricating oils, and mixtures thereof. It may range in viscosity from light distillate mineral oils to heavy lubricating oils, such as those for gas engine oil, mineral lubricating oil, motor vehicle oil, and heavy-duty diesel oil. Generally, the kinematic viscosity at 100° C. (“KV100”) of the base oil ranges from 1 to 30, such as 2 to 25 cSt, such as 5 to 20 cSt, as determined according to ASTM D445-19a, in particular, from 1.0 cSt to 10 cSt, from 1.5 cSt to 3.3 cSt, from 2.7 cSt to 8.1 cSt, from 3.0 cSt to 7.2 cSt, or from 2.5 cSt to 6.5 cSt. Generally, the high temperature high shear (HTHS) viscosity at 150° C. of the base oil ranges from 0.5 to 20 cP such as 1 to 10 cP, such as 2 to 5 cP as determined according to ASTM D4683-20.

Typically, when lubricating oil basestock(s) is used to make a concentrate, it may advantageously be present in a concentrate-forming amount to give a concentrate containing, from 5 wt % to 80 wt %, from 10 wt % to 70 wt %, or from 5 wt % to 50 wt % of active ingredient, based upon the weight of the concentrate.

Common oils useful as base oils include animal and vegetable oils (e.g., castor and lard oil), liquid petroleum oils, and hydrorefined and/or solvent-treated mineral lubricating oils of the paraffinic, naphthenic, and mixed paraffinic-naphthenic types. Oils derived from coal or shale are also useful base oils. Base stocks may be manufactured using a variety of different processes including, but not limited to, distillation, solvent refining, hydrogen processing, oligomerization, esterification, and re-refining.

Synthetic lubricating oils useful herein as base oils include hydrocarbon oils such as homopolymerized and copolymerized olefins, referred to as polyalphaolefins or PAO's or group IV base oils [according to the API EOLCS 1509 definition (American Petroleum Institute Publication 1509, see section E.1.3, 19th edition, January 2021, www.API.org)]. Examples of PAO's useful as base oils include: poly(ethylenes), copolymers of ethylene and propylene, polybutylenes, polypropylenes, propylene-isobutylene copolymers, chlorinated polybutylenes, poly(1-hexenes), poly(1-octenes), poly(1-decenes), homo- or co-polymers of C₈ to C₂₀ alkenes, homo- or co-polymers of C₈, and/or C₁₀, and/or C₁₂ alkenes, C₈/C₁₀ copolymers, C₈/C₁₀/C₁₂ copolymers, and C₁₀/C₁₂ copolymers, and the derivatives, analogues and homologues thereof.

In another embodiment, the base oil may comprise polyalphaolefins comprising oligomers of linear olefins having 6 to 14 carbon atoms, more preferably 8 to 12 carbon atoms, more preferably 10 carbon atoms having a Kinematic viscosity at 100° C. of 10 or more (as measured by ASTM D445); and preferably having a viscosity index (“VI”), as determined by ASTM D2270, of 100 or more, preferably 110 or more, more preferably 120 or more, more preferably 130 or more, more preferably 140 or more; and/or having a pour point of −5° C. or less (as determined by ASTM D97), more preferably −10° C. or less, more preferably −20° C. or less.

In another embodiment polyalphaolefin oligomers useful in the present disclosure may comprise C₂₀ to C₁₅₀₀ paraffins, preferably C₄₀ to C₁₀₀₀ paraffins, preferably C₅₀ to C₇₅₀ paraffins, preferably C₅₀ to C₅₀₀ paraffins. The PAO oligomers are dimers, trimers, tetramers, pentamers, etc., of C₅ to C₁₄ alpha-olefins in one embodiment, and C₆ to C₁₂ alpha-olefins in another embodiment, and C₈ to C₁₂ alpha-olefins in another embodiment. Suitable olefins include 1-pentene, 1-hexene, 1-heptene, 1-octene, 1-nonene, 1-decene, 1-undecene, and 1-dodecene. In one embodiment, the olefin is a combination of 1-octene, 1-decene, and 1-dodecene, or alternately may be substantially 1-decene, and the PAO is a mixture of dimers, trimers, tetramers, and pentamers (and higher) thereof. Useful PAO's are described more particularly in, for example, U.S. Pat. Nos. 5,171,908 and 5,783,531, and in Synthetic Lubricants and High-Performance Functional Fluids 1-52 (Leslie R. Rudnick & Ronald L. Shubkin, ed. Marcel Dekker, Inc. 1999).

PAO's useful in the present disclosure typically possess a number average molecular weight of from 100 to 21,000 g/mol in one embodiment, and from 200 to 10,000 g/mol in another embodiment, and from 200 to 7,000 g/mol in yet another embodiment, and from 200 to 2,000 g/mol in yet another embodiment, and from 200 to 500 g/mol in yet another embodiment. Desirable PAO's are commercially available as SpectraSyn™ Hi-Vis, SpectraSyn™ Low-Vis, SpectraSyn™ plus, SpectraSyn™ Elite PAO's (ExxonMobil Chemical Company, Houston Texas) and Durasyn PAO's from Ineos Oligomers USA LLC.

Synthetic lubricating oils useful as base oils also include hydrocarbon oils such as homopolymerized and copolymerized: alkylbenzenes (e.g., dodecylbenzenes, tetradecylbenzenes, dinonylbenzenes, di(2-ethylhexyl)benzenes); polyphenols (e.g., biphenyls, terphenyls, alkylated polyphenols); and alkylated diphenyl ethers, and alkylated diphenyl sulfides; and the derivatives, analogues, and homologues thereof.

Another suitable class of synthetic lubricating oils useful as base oils comprises the esters of dicarboxylic acids (e.g., phthalic acid, succinic acid, alkyl succinic acids and alkenyl succinic acids, maleic acid, azelaic acid, suberic acid, sebasic acid, fumaric acid, adipic acid, linoleic acid dimer, malonic acid, alkylmalonic acids, alkenyl malonic acids) reacted with a variety of alcohols (e.g., butyl alcohol, hexyl alcohol, dodecyl alcohol, 2-ethylhexyl alcohol, ethylene glycol, diethylene glycol monoether, propylene glycol). Specific examples of these esters include dibutyl adipate, di(2-ethylhexyl) sebacate, di-n-hexyl fumarate, dioctyl sebacate, diisooctyl azelate, diisodecyl azelate, dioctyl phthalate, didecyl phthalate, dieicosyl sebacate, the 2-ethylhexyl diester of linoleic acid dimer, and the complex ester formed by reacting one mole of sebacic acid with two moles of tetraethylene glycol and two moles of 2-ethylhexanoic acid.

Esters useful as synthetic oils herein also include those made from C₅ to C₁₂ monocarboxylic acids and polyols, and polyol ethers such as neopentyl glycol, trimethylolpropane, pentaerythritol, dipentaerythritol, and tripentaerythritol.

Desirable ester base oils are commercially available as Esterex™ Esters (ExxonMobil Chemical Company, Houston, Texas).

Silicon-based oils such as the polyalkyl-, polyaryl-, polyalkoxy- or polyaryloxysilicone oils and silicate oils comprise another useful class of synthetic lubricants useful herein; such oils include tetraethyl silicate, tetraisopropyl silicate, tetra-(2-ethylhexyl) silicate, tetra-(4-methyl-2-ethylhexyl)silicate, tetra-(p-tert-butyl-phenyl) silicate, hexa-(4-methyl-2-ethylhexyl)disiloxane, poly(methyl)siloxanes, and poly(methylphenyl)-siloxanes.

Other synthetic lubricating oils useful herein include liquid esters of phosphorous-containing acids (e.g., tricresyl phosphate, trioctyl phosphate, diethyl ester of decylphosphonic acid) and polymeric tetrahydrofurans.

Unrefined, refined, and re-refined oils can be used in the lubricating compositions of the present disclosure. Unrefined oils are those obtained directly from a natural or synthetic source without further purification treatment. For example, a shale oil obtained directly from retorting operations, a petroleum oil obtained directly from distillation, or an ester oil obtained directly from an esterification process and used without further treatment is considered an unrefined oil. Refined oils are similar to the unrefined oils except they have been further treated in one or more purification steps to improve one or more properties. Many such purification techniques, such as distillation, solvent extraction, acid or base extraction, filtration, and percolation are used by those in the art. Re-refined oils are oils obtained by processes similar to those used to obtain refined oils where the refining processes are applied to previously refined oils which have been previously used in service. Such re-refined oils are also referred to as reclaimed or reprocessed oils and often are additionally processed for removal of spent additive and oil breakdown products. A re-refined base oil is preferably substantially free from materials introduced through manufacturing, contamination, or previous use.

Other examples of useful base oils are gas-to-liquid (“GTL”) base oils, i.e., the base oil is an oil derived from hydrocarbons made from synthesis gas (“syn gas”) containing H2 and CO using a Fischer-Tropsch catalyst. These hydrocarbons typically require further processing in order to be useful as a base oil. For example, they may, by methods known in the art, be hydroisomerized; hydrocracked and hydroisomerized; dewaxed; or hydroisomerized and dewaxed. For further information on useful GTL base oils and blends thereof, please see U.S. Pat. No. 10,913,916 (col 4, ln 62 to col 5, ln 60) and U.S. Pat. No. 10,781,397 (col 14, ln 54 to col 15, ln 5, and col 16, ln 44 to col 17, ln 55).

In particular, oils from renewable sources, i.e., based in part on carbon and energy captured from the environment, such as biological sources, are useful herein.

The various base oils are often categorized as Group I, II, III, IV, or V according to the API EOLCS 1509 definition (American Petroleum Institute Publication 1509, see section E.1.3, 19th edition, January 2021, www.API.org). Generally speaking, Group I base stocks have a viscosity index of between about 80 to 120 and contain greater than about 0.03% sulfur and/or less than about 90% saturates. Group II base stocks have a viscosity index of between about 80 to 120 and contain less than or equal to about 0.03% sulfur and greater than or equal to about 90% saturates. Group III base stocks have a viscosity index greater than about 120 and contain less than or equal to about 0.03% sulfur and greater than about 90% saturates. Group IV base stocks includes polyalphaolefins (PAO). Group V base stocks include base stocks not included in Groups I-IV. (Viscosity index measured by ASTM D 2270, saturates is measured by ASTM D2007, and sulfur is measured by ASTM D5185, D2622, ASTM D4294, ASTM D4927, and ASTM D3120).

Base oils for use in the formulated lubricating compositions useful in the present disclosure are any one, two, three, or more of the variety of oils described herein. In desirable embodiments, base oils for use in the formulated lubricating compositions useful in the present disclosure are those described as API Group I, Group II, Group III (including Group III+), Group IV, and Group V oils and mixtures thereof, preferably API Group II, Group III, Group IV, and Group V oils and mixtures thereof, more preferably the Group III, Group III+, IV, and Group V base oils due to their exceptional volatility, stability, viscometric, and cleanliness features. Minor quantities of Group I basestock, such as the amount used to dilute additives for blending into formulated lube oil products, can be tolerated but are typically kept to a minimum, e.g., amounts only associated with their use as diluent/carrier oil for additives used on an “as-received” basis. In regard to the Group II stocks, it is more useful that the Group II base stock be in the higher quality range associated with that stock, i.e., a Group II stock having a viscosity index in the range from 100 to 120.

The base oil useful herein may be selected from any of the synthetic, natural, or re-refined oils (such as those typically used as crankcase lubricating oils for spark-ignited and compression-ignited engines). Mixtures of synthetic and/or natural and/or re-refined base oils may be used if desired. Multi-modal mixtures (such as bi- or tri-modal mixtures) of Group I, II, III, IV, and/or V base stocks may be used if desired.

The base oil or base oil blend used herein conveniently has a kinematic viscosity at 100° C. (KV100, as measured according to ASTM D445-19a, and reported in units of centistoke (cSt) or it its equivalent, mm2/s), of about 2 to about 40 cSt, alternately of 3 to 30 cSt, alternately 4 to 20 cSt at 100° C., alternately 5 to 10 cSt, alternately the base oil or base oil blend may have a kinematic viscosity at 100° C. of 2 to 20 cSt, of 2.5 to 2 cSt, and preferably of about 2.5 cSt to about 9 cSt.

The base oil or base oil blend preferably has a saturate content of at least 65 mass %, more preferably at least 75 mass %, such as at least 85 mass %, such as at least than 90 mass % as determined by ASTM D2007.

Preferably, the base oil or base oil blend will have a sulfur content of less than 1 mass %, preferably less than 0.6 mass %, most preferably less than 0.4 mass %, such as less than 0.3 mass %, based on the total mass of the lubricating composition, as measured by ASTM D5185.

In embodiments, the volatility of the base oil or base oil blend, as measured by the Noack test (ASTM D5800, procedure B), is less than or equal to 30 mass %, such as less than or equal to 25 mass %, such as less than or equal to 20 mass %, such as less than or equal to 16 mass %, such as less than or equal to 12 mass %, such as less than or equal to 10 mass %, based on the total mass of the lubricating composition.

In embodiments, the viscosity index (VI) of the base oil is at least 95, preferably at least 110, more preferably at least 120, even more preferably at least 125, most preferably from about 130 to 240, in particular from about 105 to 140 (as determined by ASTM D2270).

The base oil may be provided in a major amount, in combination with a minor amount of one or more additive components as described hereinafter, constituting a lubricant. This preparation may be accomplished by adding the additives directly to the oil or by adding the one or more additives in the form of a concentrate thereof to disperse or dissolve the additive(s). Additives may be added to the oil by any method known to those skilled in the art, either before, at the same time as, or after addition of other additives.

The base oil may be provided in a minor amount, in combination with minor amounts of one or more additive components as described hereinafter, constituting an additive concentrate. This preparation may be accomplished by adding the additives directly to the oil or by adding the one or more additives in the form of a solution, slurry or suspension thereof to disperse or dissolve the additive(s) in the oil. Additives may be added to the oil by any method known to those skilled in the art, either before, at the same time as, or after addition of other additives.

The base oil typically constitutes the major component of an engine oil lubricant composition of the present disclosure and typically is present in an amount ranging from about 50 to about 99 wt %, preferably from about 70 to about 95 wt %, and more preferably from about 80 to about 95 wt %, based on the total weight of the composition.

Typically, one or more base oils are present in the lubricating composition in an amount of 32 wt % or more, alternately 55 wt % or more, alternately 60 wt % or more, alternately 65 wt % or more, based on the total weight of the lubricating composition. Typically, one or more base oils are present in the lubricating composition at an amount of 98 wt % or less, more preferably 95 wt % or less, even more preferably 90 wt % or less. Alternately, one or more base oils are present in the lubricating composition at from 1 to 99 mass %, alternately 50 to 97 mass %, alternately to 60 to 95 mass %, alternately 70 to 95 mass %, based upon the weight of the lubricating composition.

The base oils and blends thereof described above are also useful for making concentrates as well as for making lubricants therefrom.

Concentrates constitute a convenient means of handling additives before their use, as well as facilitating solution or dispersion of additives in lubricants. When preparing a lubricant that contains more than one type of additive (sometime referred to as “additive components”), each additive may be incorporated separately, each in the form of a concentrate. In many instances, however, it is convenient to provide a so-called additive “package” (also referred to as an “addpack”) comprising one or more additives/co-additives, such as described hereinafter, in a single concentrate.

Typically, one or more base oils are present in the concentrate composition in an amount of 50 wt % or less, alternately 40 wt % or less, alternately 30 wt % or less, alternately 20 wt % or less, based on the total weight of the concentrate composition. Typically, one or more base oils are present in the concentrate composition at an amount of 0.1 to 49 mass %, alternately 5 to 40 mass %, alternately to 10 to 30 mass %, alternately 15 to 25 mass %, based upon the weight of the concentrate composition.

In embodiments, the acylation/functionalization reactions described herein may take place in the presence of base oil diluent. As a side product, functionalized base oil can be produced. The oil may become acylated and/or functionalized itself. For example, maleated base oil or aminated base oil may be present after the functionalization reactions described herein.

It is contemplated that the functionalized base oil may comprise the acylated oil.

It is contemplated that the functionalized base oil may comprise the reaction product of the acylated oil with an amine to form an amide, imide or combination thereof.

It is contemplated that the functionalized base oil may comprise both acylated oil and reaction product of the acylated oil with an amine to form an amide, imide or combination thereof.

In embodiments the lubricating oil composition and/or concentrate compositions may comprise functionalized base oil, such as acylated oil and/or reaction product of the acylated oil with an amine or alcohol to form an amide, imide, ester, or combination thereof, in an amount of 40 wt % or less, alternately 20 wt % or less, alternately 10 wt % or less, alternately 5 wt % or less, based on the total weight of the concentrate composition. Typically, one or more functionalized base oil, such as acylated oil and/or reaction product of the acylated oil with an amine or alcohol to form an amide, imide, ester, or combination thereof, are present in the concentrate at an amount of 0.01 to 40 mass %, alternately 0.1 to 20 mass %, alternately to 1 to 10 mass alternately 1.5 to 5 mass %, based upon the weight of the concentrate composition.

Typically, one or more functionalized base oil, such as acylated oil and/or reaction product of the acylated oil with an amine or alcohol to form an amide, imide, ester, or combination thereof, are present in the lubricating oil composition at an amount of 0.01 to 40 mass %, alternately 0.1 to 20 mass %, alternately to 1 to 10 mass %, alternately 1.5 to 5 mass %, based upon the weight of the lubricating oil composition.

In embodiments, functionalized oil may be present in a lubricating oil composition at 3 mass % or less, preferably 2 mass % or less, preferably 1 mass % or less, preferably at 0.1 mass % or less, preferably at 0 mass %, based upon the weight of the lubricating oil composition.

In embodiments, functionalized oil may be present in a concentrate composition at 3 mass % or less, preferably 2 mass % or less, preferably 1 mass % or less, preferably at 0.1 mass % or less, preferably at 0 mass %, based upon the weight of the concentrate composition.

In embodiments, the acylation/functionalization reactions described herein may take place in solvent-containing media. As a side product, functionalized solvent can be produced. The solvent may become acylated and/or functionalized itself. In embodiments, acylated and/or functionalized solvent may be present in a concentrate composition at 3 mass % or less, preferably 2 mass % or less, preferably 1 mass % or less, preferably at 0.1 mass % or less, preferably at 0 mass %, based upon the weight of the concentrate composition. In embodiments, functionalized solvent may be present in a lubricating oil composition at 3 mass % or less, preferably 2 mass % or less, preferably 1 mass % or less, preferably at 0.1 mass % or less, preferably at 0 mass %, based upon the weight of the lubricating oil composition.

### B. Functionalized Polymer

This disclosure relates to a functionalized polymer comprising a polymer that prior to functionalization has an Mn of about 10,000 g/mol or more, such as 20,000 g/mol or more, such as 25,000 g/mol or more, such as 30,000 g/mol or more, such as 35,000 g/mol or more (GPC-PS). Alternately, functionalized polymer comprises a polymer that prior to functionalization has an Mn of 10,000 to 300,000 g/mol, such as 20,000 to about 150,000 g/mol, such as 30,000 to about 125,000 g/mol, such as 35,000 to about 100,000 g/mol, such as 40,000 to 80,000 g/mol (GPC-PS). The polymer prior to functionalization may have an Mw/Mn of less than 2 (such as less than 1.6, such as less than 1.5, such as 1.4 or less, such as from 1 to 1.3, such as from 1.0 to 1.25, such as from 1.0 to 1.2, such as 1.0 to 1.15, such as from 1.0 to 1.1 as determined by GPC-PS). The polymer prior to functionalization may comprise repeat units of one or more olefins having 4 to 5 carbon atoms (preferably conjugated dienes having 4 to 5 carbon atoms). Prior to functionalization the C₄₋₅ polymer is preferably fully or partially saturated (such as fully or partially hydrogenated). The functionalized polymer may be obtained by reacting the C₄₋₅ polymer with an acylating agent to form acylated polymer and then reacting acylated polymer with an amine or alcohol to form an amide, imide, ester, or combination thereof. The functionalized polymer may also be obtained by reacting an acylated C₄₋₅ polymer (such as a commercially available maleated fully or partially hydrogenated C₄₋₅ polymer) with an amine to form an amide, imide or combination thereof.

This disclosure further relates to amide, imide, and/or ester functionalized saturated (such as hydrogenated) polymers of C₄₋₅ conjugated dienes described herein obtained by reacting fully or partially saturated (such as fully or partially hydrogenated) polymers of C₄₋₅ conjugated dienes having an Mw/Mn of less than 2, with an acylating agent, such as maleic acid or maleic anhydride and thereafter reacting the acylated polymer with an amine (such as a polyamine) to form an imide, amide or combination thereof.

This disclosure relates to polymers containing one or more pendant amine groups and comprising or resulting from the admixing of: at least partially (preferably completely) hydrogenated C₄₋₅ olefin polymers with an acylating agent, such as maleic acid or maleic anhydride, and thereafter reacting the acylated polymer with a polyamine to form an imide, amide or combination thereof.

In embodiments, the functionalized polymer is not prepared in aromatic solvent (such as benzene or toluene), or aromatic solvent is present at 2 wt % or less (such as 1 wt % or less, such as 0.5 wt % or less), based upon the weight of solvent, diluent, and polymer.

In embodiments, the functionalized polymer is not prepared in an alkylated naphthylenic solvent, or alkylated naphthylenic solvent is present at 5 wt % or less (such as 3 wt % or less, such as 1 wt % or less), based upon the weight of solvent, diluent, and polymer.

The polymer useful herein to prepare the functionalized polymer may be a homopolymer of butadiene, isoprene, or the like.

In embodiments, the polymer useful herein to prepare the functionalized polymer may be a homopolymer of isoprene, or a copolymer of isoprene and less than 5 mol % (such as less than 3 mol %, such as less than 1 mol %, such as less than 0.1 mol %) comonomer.

The polymer useful herein to prepare the functionalized polymer may be copolymer of isoprene and one or more of styrene, methyl-styrene, 2,3-dimethyl-butadiene, 2-methyl-1,3-pentadiene, myrcene, 3-methyl-1,3-pentadiene, 4-methyl-1,3-pentadiene, 2-phenyl-1,3-butadiene, 2-phenyl-1,3-pentadiene, 3-phenyl-1,3 pentadiene, 2,3-dimethyl-1,3-pentadiene, 2-hexyl-1,3-butadiene, 3-methyl-1,3-hexadiene, 2-benzyl-1,3-butadiene, 2-p-tolyl-1,3-butadiene 1,3-butadiene, 1,3-pentadiene, 1,3-hexadiene, 1,3-heptadiene, 2,4-heptadiene, 1,3-octadiene, 2,4-octadiene, 3,5-octadiene, 1,3-nonadiene, 2,4-nonadiene, 3,5-nonadiene, 1,3-decadiene, 2,4-decadiene, and 3,5-decadiene, [optionally the comonomer(s) are present at less than 20 mol %, less than 5 mol %, such as less than 3 mol %, such as less than 1 mol %, such as less than 0.1 mol %].

Generally, the polymerized conjugated diene polymer useful herein to prepare the functionalized polymer includes a mixture of 1,4- and 1,2-insertions (a.k.a. 2,1-insertions; for butadiene, 1,2-insertions are the same as 3,4-insertions). As measured by 1H NMR, the polymerized conjugated diene polymer useful herein to prepare the functionalized polymer contains at least about 50% of 1,4-insertions, such as at least about 75% of 1,4 insertions, such as at least about 80% of 1,4 insertions, such as at least about 90% of 1,4 insertions, such as at least about 95% of 1,4 insertions, such as at least 98% of 1,4 insertions, based upon the total of the 2,1 insertions, 1,4 insertions, and 3,4 insertions of isoprene. For purposes of this disclosure: 1) the phrase “1,4 insertion” includes 1,4 and 4,1 insertions, 2) the phrase “2,1 insertion” includes 2,1 and 1,2 insertions, and 3) the phrase “3,4 insertion” includes 3,4 and 4,3 insertions.

Optionally, styrene repeat units may be absent in the polymer useful herein to prepare the functionalized polymer. Optionally, styrene repeat units may be absent in the functionalized hydrogenated/saturated polymers.

Optionally, butadiene repeat units may be absent in the polymer useful herein to prepare the functionalized polymer. Optionally, butadiene repeat units may be absent in the functionalized hydrogenated/saturated polymers.

Optionally, the polymer useful herein to prepare the functionalized polymer may be not homopolybutylene. Optionally, the functionalized hydrogenated/saturated polymer may be not homopolybutylene.

Optionally, the polymer useful herein to prepare the functionalized polymer may be not homopolyisobutylene. Optionally, the functionalized hydrogenated/saturated polymer may be not homopolyisobutylene.

Optionally, the polymer useful herein to prepare the functionalized polymer may not be a copolymer of isoprene and butadiene. Optionally, the functionalized hydrogenated/saturated polymer may not be a copolymer of isoprene and butadiene.

The polymer useful herein to prepare the functionalized polymer and/or the functionalized polymer may be homopolymer or copolymer. The copolymer may be a random copolymer, a tapered block copolymer, a star copolymer, or a block copolymer. Block copolymers are formed from a monomer mixture comprising one or more first monomers (such as isobutylene), wherein, for example, a first monomer forms a discrete block of the polymer joined to a second discrete block of the polymer formed from a second monomer (such as butadiene). While block copolymers have substantially discrete blocks formed from the monomers, a tapered block copolymer may be composed of, at one end, a relatively pure first monomer and, at the other end, a relatively pure second monomer. The middle of the tapered block copolymer may be more of a gradient composition of the two monomers.

The polymer useful herein to prepare the functionalized polymer may typically have an Mn of 20,000 to 150,000 g/mol, alternately 20,000 to about 150,000 g/mol, alternately 30,000 to about 125,000 g/mol, alternately 35,000 to about 100,000 g/mol, alternately 40,000 to 80,000 g/mol (GPC-PS).

Polymers useful herein to prepare the functionalized polymers may typically have an Mw/Mn (as determined by GPC-PS) of 1 to 2, alternately greater than 1 to less than 2, alternately 1.1 to 1.8, alternately 1.2 to 1.5. Alternately, the polymers useful herein to prepare the functionalized polymers may typically have an Mw/Mn of 1 or greater than 1 to less than 2 (such as less than 1.8, such as less than 1.7, such as less than 1.6, such as less than 1.5, such as less than 1.4, such as less than 1.3, such as less than 1.2, such as less than 1.15, such as less than 1.12, such as less than 1.10).

The polymers used to prepare the functionalized polymers may have an Mz (as determined by GPC-PS) of 20,000 to 150,000 g/mol, alternately 20,000 to about 150,000 g/mol, alternately 30,000 to about 125,000 g/mol, alternately 35,000 to about 100,000 g/mol, alternately 40,000 to 80,000 g/mol, alternately 40,000 to 60,000 g/mol (GPC-PS).

Polymers useful herein to prepare the functionalized polymers may have a glass transition temperature (Tg) of −25° C. or less, such as −40° C. or less, such as −50° C. or less, as determined by Differential Scanning calorimetry (DSC) using a Perkin Elmer or TA Instrument Thermal Analysis System (sample is heated from ambient to 210° C. at 10° C./minute and held at 210° C. for 5 minutes, then cooled down to −40° C. at 10° C./minute and held for 5 minutes.)

Polymers useful herein to prepare the functionalized polymers typically have a residual unsaturation of less than 3%, such less than 2%, such less than 1%, such as less than 0.5%, such as less than 0.25% based upon number of double bonds in the non-hydrogenated polymer.

Polymers useful herein to prepare the functionalized polymers typically have a residual metal (such as Li, Co, and Al) content of less than 100 ppm, such less than 50 ppm, such as less than 25 ppm, such as less than 10 ppm, such as less than 5 ppm.

### Hydrogenation

The C₄₋₅ polymer useful herein to prepare the functionalized polymer can be hydrogenated partially or completely by any hydrogenating agent known to one of ordinary skill in the art. For example, a saturated or partially saturated polymer can be prepared by (a) providing a C₄₋₅ polymer containing unsaturations (such as double or triple bonds); and (b) hydrogenating at least a portion or all of the unsaturations (such as double or triple bonds) in the polymer in the presence of a hydrogenation reagent. In some embodiments, the polymer is fully hydrogenated. In some embodiments, the polymer is partially hydrogenated. In some embodiments, the polymer is saturated (hydrogenated) at 50% or more, such as 60% or more, such as 70% or more, such as 80% or more, such as 90% or more, such as 95% or more, such as 98% or more, such as 99% or more, such as from 50 to 100% saturated (hydrogenated), as determined by ozone adsorption method described in Martino N. Smits and Dirkman Hoefman, Quantative Determination of Olefinic Unsaturation by Measurement of Ozone Absorption Analytical Chemistry Vol 44, No. 9, pg. 1688, 1972, Martino N. Smits.

In embodiments, the hydrogenation reagent can be hydrogen in the presence of a hydrogenation catalyst. In some embodiments, the hydrogenation catalyst is Pd, Pd/C, Pt, PtO2, Ru(PPh3)2Cl2, Raney nickel, or a combination thereof. In embodiments, the catalyst is a Pd catalyst. In another embodiment, the catalyst is 5% Pd/C. In a further embodiment, the catalyst may comprise or be 10% Pd/C in a high-pressure reaction vessel and the hydrogenation reaction is allowed to proceed until completion. Generally, after completion, the reaction mixture can be washed, concentrated, and dried to yield the corresponding hydrogenated product. Alternatively, any reducing agent that can reduce a C═C bond to a C—C bond can also be used. For example, the olefin polymer can be hydrogenated by treatment with hydrazine in the presence of a catalyst, such as 5-ethyl-3-methyllumiflavinium perchlorate, under an oxygen atmosphere to give the corresponding hydrogenated products. The reduction reaction with hydrazine is disclosed in Imada et al., J Am. Chem. Soc., 127, pp. 14544-14545, (2005), which is incorporated herein by reference.

### Acylation

The fully or partially saturated (hydrogenated) polymer may be chemically modified (functionalized) to provide a polymer having at least one polar functional group, such as, but not limited to, halogen, epoxy, hydroxy, amino, nitrilo, mercapto, imido, carboxy, and sulfonic acid groups of combinations thereof. The functionalized polymers can be further modified to give a more desired type of functionality. In a preferred case, the fully or partially hydrogenated polymer is functionalized by a method, which includes reacting the fully or partially hydrogenated polymer with an unsaturated carboxylic acid (or derivative thereof, such as maleic anhydride) to provide an acylated polymer (which may then be further functionalized as described below).

In some embodiments, a carboxylic acid functionality or a reactive equivalent thereof is grafted onto the polymer to form an acylated polymer. An ethylenically unsaturated carboxylic acid material is typically grafted onto the polymer backbone. These materials which are attached to the polymer typically contain at least one ethylenic bond (prior to reaction) and at least one, such as two, carboxylic acid (or its anhydride) groups or a polar group which is convertible into said carboxyl groups by oxidation or hydrolysis. Maleic anhydride or a derivative thereof is suitable. It grafts onto the polymer, to give two carboxylic acid functionalities. Examples of additional unsaturated carboxylic materials include itaconic anhydride, or the corresponding dicarboxylic acids, such as maleic acid, fumaric acid and their esters, as well as cinnamic acid and esters thereof.

The ethylenically unsaturated carboxylic acid material may be grafted onto the polymer in a number of ways. It may be grafted onto the polymer in solution or in essentially pure (molten) form with or without using a radical initiator. Free-radical induced grafting of ethylenically unsaturated carboxylic acid materials may also be conducted in solvents, such as hexane or mineral oil. It may be carried out at an elevated temperature in the range of 100° C. to 250° C., e.g., 120° C. to 190° C., or 150° C. to 180° C., e.g., above 160° C.

The free-radical initiators which may be used include peroxides, hydroperoxides, and azo compounds, typically those which have a boiling point greater than about 100° C. and which decompose thermally within the grafting temperature range to provide free radicals. Representative of these free-radical initiators include azobisisobutyronitrile and 2,5-dimethyl-hex-3-yne-2,5-bis-tertiary-butyl peroxide. The initiator may be used in an amount of 0.005% to 1% by weight based on the weight of the reaction mixture solution. The grafting may be carried out in an inert atmosphere, such as under nitrogen blanketing. The resulting acylated polymer intermediate is characterized by having carboxylic acid acylating functions as a part of its structure.

In embodiments, the acylated polymer may have 2 or more anhydride groups per polymer molecule and may exhibit less than 10% gel. Alternately, the acylated polymer may have less than 2 anhydride groups per polymer molecule and may exhibit less than 10% gel. (See also col 17, ln 14-col 18, ln 11 of U.S. Pat. No. 5,429,758).

Alternately, in some embodiments, the acylated polymer may have a gel content of less than about 5 wt %, less than 3 wt %, less than 2 wt %, less than 1 wt %, less than 0.5 wt %, less than 0.1 wt %, or 0 wt %, where the gel content is measured by determining the amount of material that is extractable from the polymer by using boiling xylene (or cyclohexane) as an extractant. The percent of soluble and insoluble (gel) material in a polymer composition is determined by soaking a nominally 0.5 mm thick thin film specimen of polymer for 48 hours in cyclohexane at 23° C. or refluxing the thin film specimen in boiling xylene for one half hour, removing the solvent, weighing the dried residue and calculating the amount of soluble and insoluble (gel) material. This method is generally described in U.S. Pat. No. 4,311,628, which is incorporated herein by reference. For purposes of this disclosure, gel content is measured using boiling xylene, unless the sample is not soluble in xylene, then the cyclohexane method is used.

In embodiments, the acylated polymer may have a Saponification Number (SAP) of 5 g/KOH or more, such as 10 g/KOH or more, such as 20 g/KOH or more, such as 30 g/KOH or more, such as 50 g/KOH or more, such as 10 to 60 g/KOH, such as 20 to 40 g/KOH as determined by ASTM D94.

In embodiments, the acylated polymer composition may have less than 5 wt % unreacted acylating agent (such as maleic anhydride), such as less than 4 wt %, such as less than 3 wt %, such as less than 1 wt %, such as less than 0.5 wt %, such as less than 0.25 wt %, such as less than 0.1 wt %, based upon the weight of the acylated polymer composition (i.e., polymer, acylating agent, and diluent).

In embodiments, the acylation reactions described herein may take place in base oil diluent. As a side product, functionalized base oil can be produced. The oil may become acylated itself. For example, maleated base oil may be present after the acylation reactions described herein.

It is contemplated that the functionalized base oil may comprise the acylated oil and/or the reaction product of the acylated oil with an amine to form an amide, imide or combination thereof.

Preferably, the acylated oil and/or reaction product of the acylated oil with an amine or alcohol to form an amide, imide, ester, or combination thereof, may be present in a concentrate in an amount of 40 wt % or less, alternately 20 wt % or less, alternately 10 wt % or less, alternately 5 wt % or less, alternately 3 mass % or less, preferably 2 mass % or less, preferably 1 mass % or less, preferably at 0.1 mass % or less, preferably at 0 mass % (such as 0 to 40 mass %, alternately 0.01 to 40 mass %, alternately 0.1 to 20 mass %, alternately to 1 to 10 mass %, alternately 1.5 to 5 mass %) based upon the weight of the concentrate composition.

Preferably one or more functionalized base oils, such as acylated oil and/or reaction product of the acylated oil with an amine or alcohol to form an amide, imide, ester, or combination thereof, may be present in the lubricating oil composition at an amount of 0.01 to 40 mass %, alternately 0.1 to 20 mass %, alternately to 1 to 10 mass % alternately 1.5 to 5 mass %, (such as at 3 mass % or less, preferably 2 mass % or less, preferably 1 mass % or less, preferably at 0.1 mass % or less, preferably at 0 mass %), based upon the weight of the lubricating oil composition.

In embodiments, the acylation reactions described herein take place in solvent containing media. As a side product, acylated/functionalized solvent can be produced. In embodiments, acylated and/or functionalized solvent may be present in a concentrate composition at 3 mass % or less, preferably 2 mass % or less, preferably 1 mass % or less, preferably at 0.1 mass % or less, preferably at 0 mass %, based upon the weight of the concentrate composition. In embodiments, functionalized solvent may be present in a lubricating oil composition at 3 mass % or less, preferably 2 mass % or less, preferably 1 mass % or less, preferably at 0.1 mass % or less, preferably at 0 mass %, based upon the weight of the lubricating oil composition.

In embodiments, the acylating agent may be added in such a way as to minimize side reactions (such as reaction with base oil or other diluent present in the reaction vessel).

In embodiments, the acylating reaction may occur where the acylating agent (such as maleic acid or maleic anhydride) is added in a continuous or semi-continuous (such as intermittent) stream (such as, for example, in controlled relatively equal portions over the reaction time, or larger and/or smaller portions at different points in the reaction) to minimize functionalized base oil and other side reactions. As an example, the acylating agent may be added in a continuous fashion where the amounts of polymer and acylating agents are added in controlled stoichiometric amounts. As another example, the polymer may be added to a reaction vessel in batch fashion and the acylating agent added slowly or in a semi-continuous fashion (such as adding the acylating agent in 2 or more, such as 5 or more, such as 10 or more, such as 20 or more, such as 30 or more, such as 40 or more, such as 50 or more, such as 60 or more discrete amounts or portions). Alternately, the polymer can be added to the reaction vessel in X number of portions and the acylating agent added in 1.5× or more (such as 2× or more, such as 5× or more, such as 10× or more, such as 20× or more, such as 30× or more, such as 40× or more, such as 50× or more, such as 60× or more) number of portions. This same effect may also be achieved by diluting or concentrating a polymer solution and/or the acylating agent solution to the same or different extents.

Preferably, the acylating agent may be added in such a way as to minimize side reactions, such as in a continuous or semi-continuous fashion.

The reaction may also be run so as to minimize side reactions by using high concentrations of the polymer in diluent, such as 45 wt % or more, or 50 wt % or more, or 55 wt % or more, or 60 wt % or more in batch, semi-continuous, or continuous reactor operations. For example, the polymer (such as a hydrogenated isoprene polymer, such as hydrogenated homo-polyisoprene) may be introduced into batch, semi-continuous, or continuous reactor operations as solution or suspension (such as a slurry) in diluent (such as oil (e.g., base oil, such as a Group I, II, III, IV, and/or V base oil, such as a Group II and/or Group III base oil) or alkane solvent or diluent or a combination thereof), where the polymer may be present in the solution or suspension at 45 wt % or more (or 50 wt % or more, or 55 wt % or more, or 60 wt % or more), based upon the weight of the polymer and diluent.

In embodiments, the side reactions may be minimized by: 1) adding the acylating agent in a continuous or semi-continuous fashion, and/or 2) the polymer is introduced into batch, semi-continuous or continuous reactor operations as solution or suspension in diluent where the polymer is present at 45 wt % or more, based upon the weight of the polymer and diluent.

In embodiments, side reactions are minimized, optionally by adding the acylating agent in a continuous or semi-continuous fashion, and/or by introducing the fully or partially hydrogenated polymer (such as isoprene polymer) into batch, semi-continuous, or continuous reactor operations as solution or suspension in diluent, said solution or suspension comprising 45 wt % or more (or 50 wt % or more, or 55 wt % or more, or 60 wt % or more), of the fully or partially hydrogenated polymer, based upon the weight of the fully or partially hydrogenated polymer and diluent.

In embodiments, side reactions are minimized, optionally by adding the acylating agent in a continuous or semi-continuous fashion, and by introducing the fully or partially hydrogenated polymer (such as isoprene polymer) into batch, semi-continuous, or continuous reactor operations as solution or suspension in diluent, said solution or suspension comprising 45 wt % or more (or 50 wt % or more, or 55 wt % or more, or 60 wt % or more), of the fully or partially hydrogenated polymer, based upon the weight of the fully or partially hydrogenated polymer and diluent.

### Functionalization

In embodiments, the acylated polymer may be reacted with an alcohol or an amine to form an amide, imide, ester or combinations thereof. The reaction may consist of condensation to form an imide, an amide, a half-amide, amide-ester, diester, or an amine salt. A primary amino group will typically condense to form an amide or, in the case of maleic anhydride, an imide. It is noted the amine may have a single primary amino group or multiple primary amino groups.

Suitable amines may include one or more aromatic amines, such as amines wherein a carbon atom of the aromatic ring structure is attached directly to the amino nitrogen. The amine may also be aliphatic. In embodiments aliphatic amines can be used alone or in combination with each other or in combination with aromatic amines. The amount of aromatic amine may, in some embodiments, be a major or minor amount compared with the amount of the non-aromatic amines, or in some instances, the composition may be substantially free of aromatic amine. Alternately, the composition may be substantially free of aliphatic amine.

Examples of aromatic amines which may be used herein include one or more N-arylphenylenediamine(s) represented by the formula:

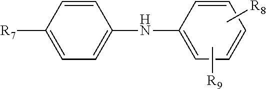

wherein R₇ is H, —NHaryl, —NHalkaryl, or a branched or straight chain hydrocarbyl radical having from about 4 to about 24 carbon atoms selected from alkyl, alkenyl, alkoxyl, aralkyl or alkaryl; R₉ is —NH₂, —(NH(CH₂)ₙ)ₘNH₂, —NHalkyl, NHaralkyl, CH₂-aryl-NH₂, in which n and m each have a value from about 1 to about 10; and R₈ is hydrogen, or alkyl, alkenyl, alkoxyl, aralkyl, or alkaryl, having from about 4 to about 24 carbon atoms.

Suitable N-arylphenylenediamines include N-phenylphenylenediamines (NPPDA), for example, N-phenyl-4,4-phenylenediamine, N-phenyl-1,3-phenylenediamine, and N-phenyl-1,2-phenylenediamine and N-naphthyl-1,4-phenylenediamine. Other derivatives of NPPDA may also be included, such as N-propyl-N′-phenylphenylenediamine.

In embodiments, the amine reacted with the acylated polymer is an amine having at least 3 or 4 aromatic groups and may be represented by the following formula:

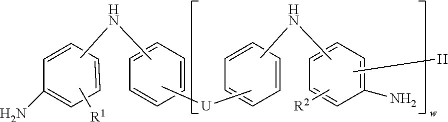

wherein independently each variable, R¹ may be hydrogen or a C₁ to C₅ alkyl group (typically hydrogen); R² may be hydrogen or a C₁ to C₅ alkyl group (typically hydrogen); U may be an aliphatic, alicyclic or aromatic group, with the proviso that when U is aliphatic, the aliphatic group may be linear or branched alkylene group containing 1 to 5, or 1 to 2 carbon atoms; and w may be 1 to 10, or 1 to 4, or 1 to 2 (typically 1).

Other examples of aromatic amines include aniline, N-alkylanilines such as N-methyl aniline, and N-butylaniline, di-(para-methylphenyl)amine, naphthylamine, 4-aminodiphenylamine, N,N-dimethylphenylenediamine, 4-(4-nitro-phenylazo)aniline (disperse orange 3), sulfamethazine, 4-phenoxyaniline, 3-nitroaniline, 4-aminoacetanilide, 4-amino-2-hydroxy-benzoic acid phenyl ester (phenyl amino salicylate), N-(4-amino-5-methoxy-2-methyl-phenyl)-benzamide (fast violet B), N-(4-amino-2,5-dimethoxy-phenyl)-benzamide (fast blue RR), N-(4-amino-2,5-diethoxy-phenyl)-benzamide (fast blue BB), N-(4-amino-phenyl)-benzamide and 4-phenylazoaniline. Suitable amines are referenced in U.S. Pat. No. 7,790,661 and are hereby incorporated by reference.

In embodiments, the compound condensing with the acylated polymer can be represented by the following formulas:

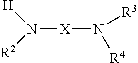

wherein X is an alkylene group containing about 1 to about 4 carbon atoms; R², R³ and R⁴ are hydrocarbyl groups.

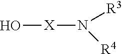

wherein X is an alkylene group containing about 1 to about 4 carbon atoms; R³ and R⁴ are hydrocarbyl groups.

Alternately, the amine may be an amine having at least 4 aromatic groups and an aldehyde (such as formaldehyde). The aromatic amine may be represented by formula:

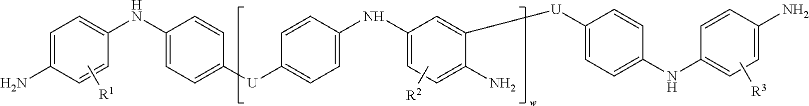

wherein, R¹ is hydrogen or a C₁₋₅ alkyl group (typically hydrogen); R² is hydrogen or a C₁₋₅ alkyl group (typically hydrogen); U is an aliphatic, alicyclic or aromatic group, optionally with the proviso that when U is aliphatic, the aliphatic group may be linear or branched alkylene group containing 1, 2, 3, 4, or 5, or 1 to 2 carbon atoms; and w is 0, 1, 2, 3, 4, 5, 6, 7, 8, or 9, such as 0, 1, 2, or 3 or 0 or 1 (typically 0). For further information on such amines see, e.g., US 2017/0073606, page 5 paragraph [0064]-[0070] and European Patent No. 2 401 348.

Examples of compounds capable of condensing with the acylating agent and further having a tertiary amino group can include but are not limited to: dimethylaminopropylamine, N,N-dimethyl-aminopropy-lamine, N,N-diethyl-aminopropylamine, N,N-dimethyl-ami-noethylamine ethylenediamine, 1,2-propylenediamine, 1,3-propylenediamine, the isomeric butylenediamines, pentanediamines, hexanediamines, heptanediamines, diethylenetriamine, dipropylenetriamine, dibutylenetriamine, triethylenetetraamine, tetraethylene pentaamine, pentaethylenehexaamine, hexamethylenetetramine, and bis (hexamethylene) triamine, the diaminobenzenes, the diaminopyridines or mixtures thereof. The compounds capable of condensing with the acylating agent and further having a tertiary amino group can further include aminoalkyl substituted heterocyclic compounds such as 1-(3-aminopropyl)imidazole and 4-(3-aminopropyl)morpholine, 1-(2-aminoethyl)piperidine, 3,3-di-amino-N-methyldipropylamine, 3′,3-aminobis(N,N-dimethylpropylamine). Another example of compounds capable of condensing with the acylating agent and having a tertiary amino group include alkanolamines including, but not limited to, triethanolamine, trimethanolamine, N,N-dimethylaminopropanol, N,N-di-ethylaminopropanol, N,N-diethylaminobutanol, N,N,N-tris (hydroxyethyl)amine, N,N,N-tris(hydroxymethyl)amine.

In embodiments, the polymer may be reacted with a polyether aromatic compound. Typically, the polyether aromatic compound will have at least two functional groups, each capable of reacting with a monocarboxylic acid or ester thereof, or dicarboxylic acid, anhydride or ester thereof, or mixtures thereof. In embodiments, the polyether aromatic compound is derived from an aromatic compound containing at least one amine group and wherein the poly ether is capable of reacting with a monocarboxylic acid or ester thereof, or dicarboxylic acid, anhydride or ester thereof.

Examples of suitable polyether aromatic amines include compounds having the following structure:

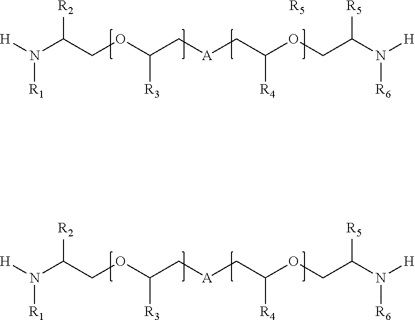

in which A represents an aromatic aminic moiety wherein the ether groups are linked through at least one amine group on the aromatic moiety; R₁ and R₆ are independently hydrogen, alkyl, alkaryl, aralkyl, or aryl or mixtures thereof; R₂, R₃, R₄, and R₅ are independently hydrogen or alkyl containing from about 1 to about 6 carbon atoms of mixtures thereof, and a and x are independently integers from about 1 to about 50.

The acylated polymer may be reacted with a polyether amine or polyether polyamine. Typical polyether amine compounds contain at least one ether unit and are chain terminated with at least one amine moiety. The polyether polyamines can be based on polymers derived from C₂-C₆ epoxides such as ethylene oxide, propylene oxide, and butylene oxide. Examples of polyether polyamines are sold under the Jeffamine™ brand and are commercially available from Hunstman Corporation.

Amines useful herein for combination with the acylated polymer include one or more of: N-phenyldiamines (such as N-phenyl-1,4-phenylenediamine, N-phenyl-p-phenylenediamine (a.k.a. 4-amino-diphenylamine, ADPA), N-phenyl-1,3-phenylenediamine, N-phenyl-1,2-phenylenediamine), nitroaniline (such as 3-nitroaniline), N-phenylethane-diamine (such as N1-phenylethane-1,2-diamine), N-aminophenylacetamide (such as N-(4-aminophenyl)acetamide), morpholinopropanamine (such as 3-morpholinopropan-1-amine), and aminoethylpiperazine (such as 1-(2-aminoethyl)piperazine).

In embodiments, the functionalization (such as amination) reactions described herein may take place in diluent (such as base oil or alkane solvent). As a side product, functionalized diluent (such as functionalized base oil) can be produced. It is contemplated that the functionalized diluent (such as functionalized base oil) may comprise reaction product of the acylated diluent (such as acylated base oil) with an amine to form an amide, imide or combination thereof.

Preferably, the reaction product of the acylated diluent (such as acylated oil) with an amine or alcohol to form an amide, imide, ester, or combination thereof, may be present in a concentrate in an amount of 40 wt % or less, alternately 20 wt % or less, alternately 10 wt % or less, alternately 5 wt % or less, alternately 3 mass % or less, preferably 2 mass % or less, preferably 1 mass % or less, preferably at 0.1 mass % or less, preferably at 0 mass % (such as 0 to 40 mass %, alternately 0.01 to 40 mass %, alternately 0.1 to 20 mass %, alternately to 1 to 10 mass %, alternately 1.5 to 5 mass %) based upon the weight of the concentrate composition.

Preferably one or more functionalized base oils, such as the reaction product of the acylated diluent (such as acylated base oil) with an amine or alcohol to form an amide, imide, ester, or combination thereof, may be present in the lubricating oil composition at an amount of 0.01 to 40 mass %, alternately 0.1 to 20 mass %, alternately to 1 to 10 mass %, alternately 1.5 to 5 mass %, (such as at 3 mass % or less, preferably 2 mass % or less, preferably 1 mass % or less, preferably at 0.1 mass % or less, preferably at 0 mass %), based upon the weight of the lubricating oil composition.

In embodiments, the functionalization (such as amination) reactions described herein may take place in solvent-containing media. As a side product, functionalized solvent can be produced. In embodiments, the functionalized solvent may be present in a concentrate composition at 3 mass % or less, preferably 2 mass % or less, preferably 1 mass % or less, preferably at 0.1 mass % or less, preferably at 0 mass %, based upon the weight of the concentrate composition. In embodiments, functionalized solvent may be present in a lubricating oil composition at 3 mass % or less, preferably 2 mass % or less, preferably 1 mass % or less, preferably at 0.1 mass % or less, preferably at 0 mass %, based upon the weight of the lubricating oil composition.

In embodiments, the acylated base oil/solvent may be removed prior to functionalization.

### Functionalized Polymer

The functionalized polymer may be a homopolymer of C₄ or C₅ olefins, such as butadiene and isoprene.

In embodiments, the functionalized polymer may be a homopolymer of isoprene, or a copolymer of isoprene and less than 5 mol % (such as less than 3 mol %, such as less than 1 mol %, such as less than 0.1 mol %) comonomer.

The functionalized polymer may comprise or be a copolymer of isoprene and one or more of styrene, methyl-styrene, 2,3-dimethyl-butadiene, 2-methyl-1,3-pentadiene, myrcene, 3-methyl-1,3-pentadiene, 4-methyl-1,3-pentadiene, 2-phenyl-1,3-butadiene, 2-phenyl-1,3-pentadiene, 3-phenyl-1,3 pentadiene, 2,3-dimethyl-1,3-pentadiene, 2-hexyl-1,3-butadiene, 3-methyl-1,3-hexadiene, 2-benzyl-1,3-butadiene, 2-p-tolyl-1,3-butadiene 1,3-butadiene, 1,3-pentadiene, 1,3-hexadiene, 1,3-heptadiene, 2,4-heptadiene, 1,3-octadiene, 2,4-octadiene, 3,5-octadiene, 1,3-nonadiene, 2,4-nonadiene, 3,5-nonadiene, 1,3-decadiene, 2,4-decadiene, and 3,5-decadiene, (optionally the comonomer(s) are present at less than 20 mol %, less than 5 mol %, such as less than 3 mol %, such as less than 1 mol %, such as less than 0.1 mol %)

In embodiments, the functionalized polymer comprises 10 (such as 9, such as 8, such as 7, such as 6, such as 5, such as 4, such as 3, such as 2, such as 1) wt %, or less, based upon the weight of the functionalized polymer, of styrene monomer.

In embodiments, styrene repeat units may be absent in the functionalized polymer.

In embodiments, the functionalized polymer may be a block or taperered block copolymer that does not comprise a styrene block.

In embodiments, the functionalized polymer may be a block or taperered block copolymer comprising (or consisting of or consisting essentially of) isoprene.

In embodiments, the functionalized polymer may be a block or taperered block copolymer comprising 50 wt % or more isoprene, based upon the weight of the copolymer.

In embodiments, the functionalized polymer may be a block or taperered block copolymer comprising (or consisting of or consisting essentially of) C₄0.5 conjugated diene, preferably comprising 50 (such as 60, such as 70, such as 80, such as 90, such as 95, such as 98) wt % or more C₄₋₅ conjugated diene, based upon the weight of the copolymer.

In embodiments, the functionalized polymer may be a copolymer comprising 50 (such as 60, such as 70, such as 80, such as 90, such as 95, such as 98) wt % or more isoprene, based upon the weight of the copolymer.

In embodiments, the functionalized polymer may be a copolymer comprising 50 (such as 60, such as 70, such as 80, such as 90, such as 95, such as 98) wt % or more butadiene, based upon the weight of the copolymer.

In embodiments, the functionalized polymer may be a copolymer comprising 50 (such as 60, such as 70, such as 80, such as 90, such as 95, such as 98) wt % or more butadiene and isoprene, based upon the weight of the copolymer.

In embodiments, the functionalized polymer may be a di-block copolymer comprising at least one block of isoprene homo- or co-polymer.

Optionally, butadiene repeat units may be absent in the functionalized polymer.

Optionally, the functionalized polymer may be not homopolyisobutylene.

Optionally, the functionalized polymer may be not a copolymer of isoprene and butadiene.

Generally, the polymerized conjugated diene in the functionalized polymer includes monomer units that have been inserted in the growing polymer chain by conjugated addition and non-conjugated addition In embodiments the functionalized polymer contains at least about 50% of by conjugated addition insertions, such as at least about 75% of by conjugated addition insertions, such as about 80% of by conjugated addition insertions, such as from about 85% to about 100% of by conjugated addition insertions, based upon the total number of by conjugated addition and non-conjugated insertions, as measured by ¹³C NMR.

The insertion of isoprene most often occurs by 2,1 insertions, 1,4 insertions (trans and cis), and 3,4 insertions of isoprene. (Measurements of the insertion geometry are determined by ¹H NMR.) As measured by ¹H NMR, the functionalized isoprene polymer contains at least about 50% of 1,4-insertions, such as at least about 75% of 1,4 insertions, such as at least about 80% of 1,4 insertions, such as at least about 90% of 1,4 insertions, such as at least about 95% of 1,4 insertions, such as at least 98% of 1,4 insertions, based upon the total of the 2,1 insertions, 1,4 insertions, and 3,4 insertions of isoprene. For purposes of this disclosure: 1) the phrase “1,4 insertion” includes 1,4 and 4,1 insertions, 2) the phrase “2,1 insertion” includes 2,1 and 1,2 insertions, and 3) the phrase “3,4 insertion” includes 3,4 and 4,3 insertions.

The functionalized polymer may be homopolymer or copolymer. Optionally, the functionalized polymer comprises a homopolymer or copolymer of isoprene. The copolymer may be a random copolymer, a tapered block copolymer, a star copolymer, or a block copolymer.

The functionalized polymer may typically have an Mn of 20,000 to 150,000 g/mol, alternately 20,000 to about 150,000 g/mol, alternately 30,000 to about 125,000 g/mol, alternately 35,000 to about 100,000 g/mol, alternately 40,000 to 80,000 g/mol (GPC-PS).

The polymer prior to functionalization may typically have an Mn/Mw (GPC-PS) of 1.0 to 2, such as 1.1 to 1.5, such as 1.1 to 1.3, such as 1.1 to 1.2. As functionalization occurs, Mw/Mn broadening may occur.

The functionalized polymer may typically have an Mw/Mn (GPC-PS) of 1 to 3, alternately 1 to 2, alternately greater than 1 to less than 2, alternately 1.05 to 1.9, alternately 1.10 to 1.8, alternately 1.10 to 1.7, alternately 1.12 to 1.6, alternately 1.13 to 1.5, alternately 1.15 to 1.4, alternately 1.15 to 1.3. Alternately, the functionalized polymer may typically have an Mw/Mn of 1 or greater than 1 to less than 2 (such as less than 1.8, such as less than 1.7, such as less than 1.6, such as less than 1.4, such as less than 1.2, such as less than 1.15, such as less than 1.12, such as less than 1.10).

In embodiments, the functionalized polymer may have a Saponification Number (SAP) of 25 (such as 28, such as 30, such as 32, such as 34) mgKOH/g or more, as determined by ASTM D94.

In embodiments, the functionalized polymer may contribute 17% or more (such as 20% or more, such as 17 to 40%, such as 20 to 30%) to the Saponification Number of the lubricating oil composition.

In embodiments, the functionalized polymer may have an average functionality of 1.4 to 20 FG grafts/polymer chain, such as 1.4 to 15 FG grafts/polymer chain, such as 3 to 12.5 FG grafts/polymer chain, such as 4 to 10 FG grafts/polymer chain, as determined by GPC-PS.

The functionalized polymer may have an average functionality of 15 (such as 14, 13, 12, 11, 10, 9, 8, 7, or 6) or less FG grafts/polymer chain, as determined by GPC-PS.

The functionalized polymer may have an average functionality of 1 (such as 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, or 4.0) or more FG grafts/polymer chain, as determined by GPC-PS.

The functionalized polymer may have an average functionality from 1 (such as 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, or 4.0) to 15 (such as 14, 13, 12,11, 10, 9, 8, 7, or 6) FG grafts/polymer chain, as determined by GPC-PS.

In embodiments, the functionalized polymer may have an aromatic content of 5% or less, such as 3% ore less, such as 1% or less, such as 0%, based upon the weight of the polymer.

In embodiments, the functionalized polymer may comprise acylated polymers of branched C₄₋₅ monomers having an Mn of 20,000 to 500,000 g/mol having an Mw/Mn of 2 or less, such as from 1 to 2.0, as determined by GPC-PS.

In embodiments, the functionalized polymer may have a number average molecular weight (Mn) of 20,000 (such as 25,000, such as 30,000, such as 35,000 such as 40,000) g/mol or more, as determined by GPC-PS.

In embodiments, the functionalized polymer may have a weight average molecular weight (Mw) of 50,000 (such as 40,000, such as 35,000) g/mol or less, as determined by GPC-PS. In embodiments, the functionalized polymer may have a weight average molecular weight (Mw) of 1000 to 50,000 g/mol, such as 5000 to 40,000 g/mol as determined by GPC-PS.

In embodiments, the functionalized polymer may have a z average molecular weight (Mz) of 5000 to 150,000 g/mol, such as 10,000 to 150,000 g/mol, such as 15,000 to 70,000 g/mol, such as 20,000 to 150,000 g/mol, alternately 20,000 to about 150,000 g/mol, alternately 30,000 to about 125,000 g/mol, alternately 35,000 to about 100,000 g/mol, alternately 40,000 to 80,000 g/mol, alternately 40,000 to 60,000 g/mol (GPC-PS).

In embodiments, the functionalized polymer may have a gel content of less than about 5 wt %, less than 3 wt %, less than 2 wt %, less than 1 wt %, less than 0.5 wt %, less than 0.1 wt %, or 0 wt %, where the gel content is measured by determining the amount of material that is extractable from the polymer by using boiling xylene (or cyclohexane) as an extractant. The percent of soluble and insoluble (gel) material in a polymer composition is determined as described herein.

In embodiments, the functionalized polymer may have a Functionality Distribution (Fd) value of 3.5 or less (such as 3.4 or less, such as from 1 to 3.3, such as from 1.1 to 3.2, such as from 1.2 to 3.0, such as 1.4 to 2.9, as determined by GPC-PS). Functionality Distribution (Fd) value is determined as set out in the Example section below and an average functionality of 1.4 to 20 FG grafts/polymer chain, such as 1.4 to 15 FG grafts/polymer chain, such as 3 to 12.5 FG grafts/polymer chain, such as 4 to 10 FG grafts/polymer chain, as determined by GPC-PS.

This disclosure relates to amide, imide, and/or ester functionalized hydrogenated/saturated polymers comprising (consisting essentially of or consisting of) C₄₋₅ olefins having an Mw/Mn of less than 2, a Functionality Distribution (Fd) value of 3.5 or less (such as 3.4 or less, such as from 1 to 3.3, such as from 1.1 to 3.2, such as from 1.2 to 3.0, such as 1.4 to 2.9, as determined by GPC-PS), and wherein, if the polymer prior to functionalization is a C₄ olefin polymer such as polyisobutylene, polybutadiene, or a copolymer thereof (preferably a polyisobutylene or a copolymer of isobutylene and butadiene), then the C₄ olefin polymer has an Mn of 10,000 g/mol or more (GPC-PS), and if the polymer prior to functionalization is a C₄/C₅ copolymer of isoprene and butadiene, then the Mn of the copolymer is greater than 25,000 Mn (GPC-PS).

This disclosure also relates to amide, imide, and/or ester functionalized hydrogenated/saturated polymers comprising 90 mol % or more isoprene repeat units, having an Mw/Mn of less than 2, a Functionality Distribution (Fd) value of 3.5 or less (such as 3.4 or less, such as from 1 to 3.3, such as from 1.1 to 3.2, such as from 1.2 to 3.0, such as 1.4 to 2.9, as determined by GPC-PS), and wherein the polymer prior to functionalization has an Mn of 30,000 g/mol or more (GPC-PS).

This disclosure also relates to amide, imide, and/or ester functionalized hydrogenated/saturated homopolymers of isoprene having an Mw/Mn of less than 2, a Functionality Distribution (Fd) value of 3.5 or less (such as 3.4 or less, such as from 1 to 3.3, such as from 1.1 to 3.2, such as from 1.2 to 3.0, such as 1.4 to 2.9, as determined by GPC-PS), and wherein the polymer prior to functionalization has an Mn of 30,000 g/mol or more (as determined by GPC-PS).

The lubricating composition according to the present disclosure may further comprise one or more additives such as detergents, friction modifiers, antioxidants, pour point depressants, anti-foam agents, viscosity modifiers, dispersants, corrosion inhibitors, antiwear agents, extreme pressure additives, demulsifiers, seal compatibility agents, additive diluent base oils, etc. Specific examples of such additives are described in, for example, Kirk-Othmer Encyclopedia of Chemical Technology, third edition, volume 14, pp. 477-526, and several are discussed in further detail below.

### C. Detergents

The lubricating composition may comprise one or more metal detergents (such as blends of metal detergents) also referred to as a “detergent additive.” Metal detergents typically function both as detergents to reduce or remove deposits and as acid neutralizers or rust inhibitors, thereby reducing wear and corrosion and extending engine life. Detergents generally comprise a polar head with a long hydrophobic tail, with the polar head comprising a metal salt of an acidic organic compound. The salts may contain a substantially stoichiometric amount of the metal in which case they are usually described as normal or neutral salts, and would typically have a total base number (“TBN” as measured by ASTM D2896) of up to 150 mgKOH/g, such as from 0 to 80 (or 5-30) mgKOH/g. A large amount of a metal base may be incorporated by reacting excess metal compound (e.g., an oxide or hydroxide) with an acidic gas (e.g., carbon dioxide). Such detergents, sometimes referred to as overbased, may have a TBN of 100 mgKOH/g or more (such as 200 mgKOH/g or more), and typically will have a TBN of 250 mgKOH/g or more, such as 300 mgKOH/g or more, such as from 200 to 800 mgKOH/g, 225 to 700 mgKOH/g, 250 to 650 mgKOH/g, or 300 to 600 mgKOH/g, such as 150 to 650 mgKOH/g.

Suitable detergents include, oil-soluble neutral and overbased sulfonates, phenates, sulfurized phenates, thiophosphonates, salicylates, naphthenates and other oil-soluble carboxylates of a metal, particularly the alkali metals (Group 1 metals, e.g., Li, Na, K, Rb) or alkaline earth metals (Group 2 metals, e.g., Be, Mg, Ca, Sr, Ba), particularly, sodium, potassium, lithium, calcium, and magnesium, such as Ca and/or Mg. Furthermore, the detergent may comprise a hybrid detergent comprising any combination of sodium, potassium, lithium, calcium, or magnesium salts of sulfonates, phenates, sulfurized phenates, thiophosphonates, salicylates, and naphthenates or other oil-soluble carboxylates of a Group 1 and/or 2 metal.

Preferably, the detergent additive(s) useful in the present disclosure comprises calcium and/or magnesium metal salts. The detergent may be a calcium and/or magnesium carboxylate (e.g., salicylates), sulfonate, or phenate detergent. More preferably, the detergent additives are selected from magnesium salicylate, calcium salicylate, magnesium sulfonate, calcium sulfonate, magnesium phenate, calcium phenate, and hybrid detergents comprising two, three, four, or more of more of these detergents and/or combinations thereof.

The metal-containing detergent may also include “hybrid” detergents formed with mixed surfactant systems including phenate and/or sulfonate components, e.g., phenate/salicylates, sulfonate/phenates, sulfonate/salicylates, sulfonates/phenates/salicylates, as described, for example, in U.S. Pat. Nos. 6,429,178; 6,429,179; 6,153,565; and 6,281,179. Where, for example, a hybrid sulfonate/phenate detergent is employed, the hybrid detergent would be considered equivalent to amounts of distinct phenate and sulfonate detergents introducing like amounts of phenate and sulfonate soaps, respectively.

The overbased metal-containing detergent may be sodium salts, calcium salts, magnesium salts, or mixtures thereof of the phenates, sulfur-containing phenates, sulfonates, salixarates, and salicylates. Overbased phenates and salicylates typically have a total base number of 180 to 650 mgKOH/g, such as 200 to 450 TBN mgKOH/g. Overbased sulfonates typically have a total base number of 250 to 600 mgKOH/g, or 300 to 500 mgKOH/g. In embodiments, the sulfonate detergent may be predominantly a linear alkylbenzene sulfonate detergent having a metal ratio of at least 8 as is described in paragraphs [0026] to [0037] of US Patent Application Publication No. 2005/065045 (and granted as U.S. Pat. No. 7,407,919). The overbased detergent may be present at 0 wt % to 15 wt %, or 0.1 wt % to 10 wt %, or 0.2 wt % to 8 wt %, or 0.2 wt % to 3 wt %, based upon of the lubricating composition. For example, in a heavy-duty diesel engine, the detergent may be present at 2 wt % to 3 wt % of the lubricating composition. For a passenger car engine, the detergent may be present at 0.2 wt % to 1 wt % of the lubricating composition.

The detergent additive(s) may comprise one or more magnesium sulfonate detergents. The magnesium detergent may be a neutral salt or an overbased salt. Suitably the magnesium detergent is an overbased magnesium sulfonate having a TBN of from 80 to 650 mgKOH/g (ASTM D2896), such as 200 to 500 mgKOH/g, such as 240 to 450 mgKOH/g.

Alternately, the detergent additive(s) is a magnesium salicylate. Suitably the magnesium detergent is a magnesium salicylate having TBN of from 30 to 650 mgKOH/g (ASTM D2896), such as 50 to 500 mgKOH/g, such as 200 to 500 mgKOH/g, such as 240 to 450 mgKOH/g or alternately of 150 mgKOH/g or less, such as 100 mgKOH/g or less.

Alternately, the detergent additive(s) is a combination of magnesium salicylate and magnesium sulfonate.

The magnesium detergent provides the lubricating composition thereof with from 200-4000 ppm of magnesium atoms, suitably from 200-2000 ppm, from 300 to 1500 or from 450-1200 ppm of magnesium atoms (ASTM D5185).

The detergent composition may comprise (or consist of) a combination of one or more magnesium sulfonate detergents and one or more calcium salicylate detergents.

The combination of one or more magnesium sulfonate detergents and one or more calcium salicylate detergents provides the lubricating composition thereof with: 1) from 200-4000 ppm of magnesium atoms, suitably from 200-2000 ppm, from 300 to 1500 ppm or from 450-1200 ppm of magnesium atoms (ASTM D5185), and 2) at least 500 ppm, preferably at least 750 ppm, more preferably at least 900 ppm of atomic calcium, such as from 500-4000 ppm, preferably from 750-3000 ppm, more preferably from 900-2000 ppm atomic calcium (ASTM D5185).

The detergent may comprise one or more calcium detergents such as calcium carboxylate (e.g., salicylate), sulfonate, or phenate detergent.

Suitably the calcium detergent has a TBN of from 30 to 700 mgKOH/g (ASTM D2896), such as 50 to 650 mgKOH/g, such as 200 to 500 mgKOH/g, such as 240 to 450 mgKOH/g or alternately of 150 mgKOH/g or less, such as 100 mgKOH/g or less, or 200 mgKOH/g or more, or 300 mgKOH/g or more, or 350 mgKOH/g or more.

Suitably, the calcium detergent is a calcium salicylate, sulfonate, or phenate having a TBN of from 30 to 700 mgKOH/g, 30 to 650 mgKOH/g (ASTM D2896), such as 50 to 650 mgKOH/g, such as 200 to 500 mgKOH/g, such as 240 to 450 mgKOH/g or alternately of 150 mgKOH/g or less, such as 100 mgKOH/g or less, or 200 mgKOH/g or more, or 300 mgKOH/g or more, or 350 mgKOH/g or more.

Calcium detergent is typically present in amount sufficient to provide at least 500 ppm, preferably at least 750 more preferably at least 900 ppm atomic calcium to the lubricating oil composition (ASTM D5185). If present, any calcium detergent is suitably present in amount sufficient to provide no more than 4000 ppm, preferably no more than 3000 ppm, more preferably no more than 2000 ppm atomic calcium to the lubricating oil composition (ASTM D5185). If present, any calcium detergent is suitably present in amount sufficient to provide at from 500-4000 ppm, preferably from 750-3000 ppm more preferably from 900-2000 ppm atomic calcium to the lubricating oil composition (ASTM D5185).

Suitably the total atomic amount of metal from detergent in the lubrication composition according to all aspects of the disclosure is no more than 5000 ppm, preferably no more than 4000 pm and more preferably no more than 2000 ppm (ASTM D5185). The total amount of atomic metal from detergent in the lubrication oil composition according to all aspects of the disclosure is suitably at least 500 ppm, preferably at least 800 ppm and more preferably at least 1000 ppm (ASTM D5185). The total amount of atomic metal from detergent in the lubrication oil composition according to all aspects of the disclosure is suitably from 500 to 5000 ppm, preferably from 500 to 3000 ppm and more preferably from 500 to 2000 ppm (ASTM D5185).

Sulfonate detergents may be prepared from sulfonic acids which are typically obtained by the sulfonation of alkyl substituted aromatic hydrocarbons, such as those obtained from the fractionation of petroleum or by the alkylation of aromatic hydrocarbons. Examples include those obtained by alkylating benzene, toluene, xylene, naphthalene, diphenyl, or their halogen derivatives such as chlorobenzene, chlorotoluene, and chloronaphthalene. The alkylation may be carried out in the presence of a catalyst with alkylating agents having from about 3 to more than 70 carbon atoms. The alkaryl sulfonates usually contain from about 9 to about 80 or more carbon atoms, preferably from about 16 to about 60 carbon atoms per alkyl substituted aromatic moiety. The oil soluble sulfonates or alkaryl sulfonic acids may be neutralized with oxides, hydroxides, alkoxides, carbonates, carboxylate, sulfides, hydrosulfides, nitrates, borates and ethers of the metal. The amount of metal compound is chosen having regard to the desired TBN of the final product, but typically ranges from about 100 to 220 mass % (preferably at least 125 mass %) of that stoichiometrically required.

Metal salts of phenols and sulfurized phenols are prepared by reaction with an appropriate metal compound such as an oxide or hydroxide and neutral or overbased products may be obtained by methods well known in the art. Sulfurized phenols may be prepared by reacting a phenol with sulfur or a sulfur-containing compound such as hydrogen sulfide, sulfur monohalide, or sulfur dihalide, to form products which are generally mixtures of compounds in which 2 or more phenols are bridged by sulfur-containing bridges.

Carboxylate detergents, e.g., salicylates, can be prepared by reacting an aromatic carboxylic acid (such as a C₅₋₁₀₀, C₉₋₃₀, C₁₄₋₂₄ alkyl-substituted hydroxy-benzoic acid) with an appropriate metal compound such as an oxide or hydroxide and neutral or overbased products may be obtained by methods well known in the art. The aromatic moiety of the aromatic carboxylic acid can contain heteroatoms, such as nitrogen and oxygen. Preferably, the moiety contains only carbon atoms; more preferably the moiety contains six or more carbon atoms; for example, benzene is a preferred moiety. The aromatic carboxylic acid may contain one or more aromatic moieties, such as one or more benzene rings, either fused or connected via alkylene bridges.

Preferred substituents in oil-soluble salicylic acids are alkyl substituents. In alkyl-substituted salicylic acids, the alkyl groups advantageously contain 5 to 100, preferably 9 to 30, especially 14 to 20, carbon atoms. Where there is more than one alkyl group, the average number of carbon atoms in all of the alkyl groups is preferably at least 9 to ensure adequate oil solubility.

In embodiments, the ratio of atomic detergent metal to atomic molybdenum in the lubricating oil composition may be less than 3:1, such as less than 2:1.

Further, as metal organic and inorganic base salts, which are used as detergents can contribute to the sulfated ash content of a lubricating oil composition, in embodiments of the present disclosure, the amounts of such additives are minimized. In order to maintain a low sulfur level, salicylate detergents can be used and the lubricating composition herein may comprise one or more salicylate detergents (said detergents are preferably used in amounts in the range of 0.05 to 20.0 wt %, more preferably from 1.0 to 10.0 wt % and most preferably in the range of from 2.0 to 5.0 wt %, based on the total weight of the lubricating composition).

The total sulfated ash content of the lubricating composition herein is typically not greater than 2.0 wt %, alternately at a level of not greater than 1.0 wt % and alternately at a level of not greater than 0.8 wt %, based on the total weight of the lubricating composition as determined by ASTM D874.

Furthermore, it is useful that each of the detergents, independently, have a TBN (total base number) value in the range of from 10 to 700 mgKOH/g, 10 to 500 mgKOH/g, alternately in the range of from 100 to 650, alternately in the range of from 10 to 500 mgKOH/g, alternately in the range of from 30 to 350 mgKOH/g, and alternately in the range of from 50 to 300 mgKOH/g, as measured by ISO 3771.

The sulfonate detergents (such as Ca and/or Mg sulfonate detergents) may be present in an amount to deliver 0.1 wt % to 1.5 wt %, or 0.15 to 1.2 wt %, or 0.2 wt % to 0.9 wt % sulfonate soap to the lubricant composition.

The salicylate detergents (such as Ca and/or Mg salicylate detergents) are present in an amount to deliver 0.3 wt % to 1.4 wt %, or 0.35 wt % to 1.2 wt %, or 0.4 wt % to 1.0 wt % salicylate soap to the lubricant composition.

The sulfonate soap may be present in an amount 0.2 wt % to 0.8 wt % of the lubricant composition, and the salicylate soap may be present in an amount 0.3 wt % to 1.0 wt % of the lubricant composition.

The total of all alkaline earth metal detergent soap may be present in an amount 0.6 wt % to 2.1 wt %, or 0.7 wt % to 1.4 wt % of the lubricant composition.

Typically, lubricating compositions formulated for use in heavy-duty diesel engines comprise detergents at from about 0.1 to about 10 mass %, alternately from about 0.5 to about 7.5 mass %, alternately from about 1 to about 6.5 mass %, based on the lubricating composition.

Typically, lubricating compositions formulated for use in a passenger-car engines comprise detergents at from about 0.1 to about 10 mass %, alternately from about 0.5 to about 7.5 mass %, alternately from about 1 to about 6.5 mass %, based on the lubricating composition.

Typically, lubricating compositions formulated for use in a drive train (e.g., transmissions) comprise detergents at from about 0.1 to about 10 mass %, alternately from about 0.5 to about 7.5 mass %, alternately from about 2 to about 6.5 mass %, based on the lubricating composition.

### D. Friction Modifiers

A friction modifier is any material or materials that can alter the coefficient of friction of a surface lubricated by any lubricant or fluid-containing such material(s). Friction modifiers, also known as friction reducers, or lubricity agents or oiliness agents, and other such agents that change the ability of base oils, formulated lubricating compositions, or functional fluids, to modify the coefficient of friction of a lubricated surface may be effectively used in combination with the base oils or lubricating compositions of the present disclosure if desired. Friction modifiers that lower the coefficient of friction are particularly advantageous in combination with the base oils and lubricating compositions of this disclosure.

Illustrative friction modifiers may include, for example, organometallic compounds or materials, or mixtures thereof. Illustrative organometallic friction modifiers useful in the lubricating oil formulations of this disclosure include, for example, tungsten and/or molybdenum compounds, such as molybdenum amine, molybdenum diamine, an organotungstenate, a molybdenum dithiocarbamate, molybdenum dithiophosphates, molybdenum amine complexes, molybdenum carboxylates, and the like, and mixtures thereof. Examples of useful molybdenum-containing compounds may conveniently include molybdenum dithiocarbamates, trinuclear molybdenum compounds, for example, as described in PCT Publication No. WO 98/26030, sulfides of molybdenum and molybdenum dithiophosphate.

Other known friction modifiers comprise oil-soluble organo-molybdenum compounds. Such organo-molybdenum friction modifiers may also provide antioxidant and antiwear credits to a lubricating oil composition. Examples of such oil-soluble organo-molybdenum compounds include dithiocarbamates, dithiophosphates, dithiophosphinates, xanthates, thioxanthates, sulfides, and the like, and mixtures thereof. Particularly preferred are molybdenum dithiocarbamates, dialkyldithiophosphates, alkyl xanthates and alkylthioxanthates.

Additionally, the molybdenum compound may be an acidic molybdenum compound. These compounds will react with a basic nitrogen compound as measured by ASTM test D664 or D2896 titration procedure and are typically hexavalent. Included are molybdic acid, ammonium molybdate, sodium molybdate, potassium molybdate, and other alkali metal molybdates and other molybdenum salts, e.g., hydrogen sodium molybdate, MoOC₁₄, MoO2Br2, Mo2O3C₁₆, molybdenum trioxide or similar acidic molybdenum compounds.

Among the molybdenum compounds useful in the compositions of this disclosure are organo-molybdenum compounds of the formula Mo(R″OCS₂)₄ and Mo(R″SCS₂)₄, wherein R″ is an organo group selected from the group consisting of alkyl, aryl, aralkyl and alkoxyalkyl, generally of from 1 to 30 carbon atoms, and preferably 2 to 12 carbon atoms and most preferably alkyl of 2 to 12 carbon atoms. Especially preferred are the dialkyldithiocarbamates of molybdenum.

Another group of organo-molybdenum compounds useful in the lubricating compositions of this disclosure are trinuclear molybdenum compounds, especially those of the formula Mo3 SkLnQz and mixtures thereof wherein the L are independently selected ligands having organo groups with a sufficient number of carbon atoms to render the compound soluble or dispersible in the oil, n is from 1 to 4, k varies from 4 to 7, Q is selected from the group of neutral electron-donating compounds such as water, amines, alcohols, phosphines, and ethers, and z ranges from 0 to 5 and includes non-stoichiometric values. At least 21 carbon atoms should be present among all the ligand/organo groups, such as at least 25, at least 30, or at least 35, carbon atoms.

Lubricating oil compositions useful in all aspects of the present disclosure preferably contain at least 10 ppm, at least 30 ppm, at least 40 ppm and more preferably at least 50 ppm molybdenum. Suitably, lubricating oil compositions useful in all aspects of the present disclosure contain no more than 1000 ppm, no more than 750 ppm, or no more than 500 ppm of molybdenum. Lubricating oil compositions useful in all aspects of the present disclosure preferably contain from 10 to 1000, such as 30 to 750 or 40 to 500, ppm of molybdenum (measured as atoms of molybdenum).

For more information or useful friction modifiers containing Mo, please see U.S. Pat. No. 10,829,712 (col 8, ln 58 to col 11, In 31).

Ashless friction modifiers may be present in the lubricating oil compositions of the present disclosure and are known generally and include esters formed by reacting carboxylic acids and anhydrides with alkanols and amine-based friction modifiers. Other useful friction modifiers generally include a polar terminal group (e.g., carboxyl or hydroxyl) covalently bonded to an oleophilic hydrocarbon chain. Esters of carboxylic acids and anhydrides with alkanols are described in U.S. Pat. No. 4,702,850. Examples of other conventional organic friction modifiers are described by M. Belzer in the “Journal of Tribology” (1992), Vol. 114, pp. 675-682 and M. Belzer and S. Jahanmir in “Lubrication Science” (1988), Vol. 1, pp. 3-26. Typically, the total amount of organic ashless friction modifier in a lubricant according to the present disclosure does not exceed 5 mass %, based on the total mass of the lubricating oil composition and preferably does not exceed 2 mass % and more preferably does not exceed 0.5 mass %.

Illustrative friction modifiers useful in the lubricating compositions described herein include, for example, alkoxylated fatty acid esters, alkanolamides, polyol fatty acid esters, borated glycerol fatty acid esters, fatty alcohol ethers, and mixtures thereof.

Illustrative alkoxylated fatty acid esters include, for example, polyoxyethylene stearate, fatty acid polyglycol ester, and the like. These can include polyoxypropylene stearate, polyoxybutylene stearate, polyoxyethylene isosterate, polyoxypropylene isostearate, polyoxyethylene palmitate, and the like.

Illustrative alkanolamides include, for example, lauric acid diethylalkanolamide, palmic acid diethylalkanolamide, and the like. These can include oleic acid diethyalkanolamide, stearic acid diethylalkanolamide, oleic acid diethylalkanolamide, polyethoxylated hydrocarbylamides, polypropoxylated hydrocarbylamides, and the like.

Illustrative polyol fatty acid esters include, for example, glycerol monooleate, saturated mono-, di-, and tri-glyceride esters, glycerol monostearate, and the like. These can include polyol esters, hydroxyl-containing polyol esters, and the like.

Illustrative borated glycerol fatty acid esters include, for example, borated glycerol monooleate, borated saturated mono-, di-, and tri-glyceride esters, borated glycerol monosterate, and the like. In addition to glycerol polyols, these can include trimethylolpropane, pentaerythritol, sorbitan, and the like. These esters can be polyol monocarboxylate esters, polyol dicarboxylate esters, and on occasion polyoltricarboxylate esters. Preferred can be the glycerol monooleates, glycerol di-oleates, glycerol tri-oleates, glycerol mono-oleates, glycerol di-stearates, and glycerol tri-stearates and the corresponding glycerol mono-palmitates, glycerol di-palmitates, and glycerol tri-palmitates, and the respective isostearates, linoleates, and the like. Ethoxylated, propoxylated, and/or butoxylated fatty acid esters of polyols, especially using glycerol as underlying polyol are useful herein.

Illustrative fatty alcohol ethers include, for example, stearyl ether, myristyl ether, and the like. Alcohols, including those that have carbon numbers from C₃ to C₅₀, can be ethoxylated, propoxylated, or butoxylated to form the corresponding fatty alkyl ethers. The underlying alcohol portion can preferably be stearyl, myristyl, C₁₁-C₁₃ hydrocarbon, oleyl, isosteryl, and the like.

Useful concentrations of friction modifiers may range from 0.01 wt % to 5 wt %, or about 001 wt % to about 2.5 wt %, or about 0.05 wt % to about 1.5 wt %, or about 0.051 wt % to about 1 wt %. Concentrations of molybdenum-containing materials are often described in terms of Mo metal concentration. Advantageous concentrations of Mo may range from 25 ppm to 700 ppm or more, and often with a preferred range of 50-200 ppm. Friction modifiers of all types may be used alone or in mixtures with the materials of this disclosure. Often mixtures of two or more friction modifiers, or mixtures of friction modifier(s) with alternate surface-active material(s), are also desirable. For example, combinations of Mo-containing compounds with polyol fatty acid esters, such as glycerol mono-oleate are useful herein.

### E. Antioxidants

Antioxidants retard the oxidative degradation of base oils during service. Such degradation may result in deposits on metal surfaces, the presence of sludge, a viscosity increase in a lubricant, and the like. A wide variety of oxidation inhibitors that are useful in lubricating oil compositions. See Lubricants and Related Products, Klamann, Wiley VCH, 1984; U.S. Pat. Nos. 4,798,684 and 5,084,197, for example.

Useful antioxidants include hindered phenols. These phenolic antioxidants may be ashless (metal-free) phenolic compounds or neutral or basic metal salts of certain phenolic compounds. Typical phenolic antioxidant compounds are the hindered phenolics, which contain a sterically hindered hydroxyl group, and these include those derivatives of dihydroxy aryl compounds in which the hydroxyl groups are in the o- or p-position to each other. Typical phenolic antioxidants include the hindered phenols substituted with C₆₊ alkyl groups and the alkylene coupled derivatives of these hindered phenols. Examples of phenolic materials of this type 2-t-butyl-4-heptyl phenol; 2-t-butyl-4-octyl phenol; 2-t-butyl-4-dodecyl phenol; 2,6-di-t-butyl-4-heptyl phenol; 2,6-di-t-butyl-4-dodecyl phenol; 2-methyl-6-t-butyl-4-heptyl phenol; and 2-methyl-6-t-butyl-4-dodecyl phenol. Other useful hindered mono-phenolic antioxidants may include, for example, hindered 2,6-di-alkyl-phenolic proprionic ester derivatives. Bis-phenolic antioxidants may also be advantageously used herein. Examples of ortho-coupled phenols include: 2,2′-bis(4-heptyl-6-t-butyl-phenol); 2,2′-bis(4-octyl-6-t-butyl-phenol); and 2,2′-bis(4-dodecyl-6-t-butyl-phenol). Para-coupled bisphenols include, for example, 4,4′-bis(2,6-di-t-butyl-phenol) and 4,4′-methylene-bis(2,6-di-t-butyl-phenol).

Effective amounts of one or more catalytic antioxidants may also be used. The catalytic antioxidants comprise an effective amount of a) one or more oil soluble polymetal organic compounds; and, effective amounts of b) one or more substituted N,N′-diaryl-o-phenylenediamine compounds or c) one or more hindered phenol compounds; or a combination of both b) and c). Catalytic antioxidants useful herein are more fully described in U.S. Pat. No. 8,048,833.

Non-phenolic oxidation inhibitors, which may be used include aromatic amine antioxidants and these may be used either as such or in combination with phenolics. Typical examples of non-phenolic antioxidants include: alkylated and non-alkylated aromatic amines such as aromatic monoamines of the formula R₈R₉R₁₀N, where R₈ is an aliphatic, aromatic or substituted aromatic group, R₉ is an aromatic or a substituted aromatic group, and R₁₀ is H, alkyl, aryl or R₁₁S(O)XR₁₂ where R₁₁ is an alkylene, alkenylene, or aralkylene group, R₁₂ is an alkyl group, or an alkenyl, aryl, or alkaryl group, and x is 0, 1, or 2. The aliphatic group R₅ may contain from 1 to about 20 carbon atoms, and preferably contains from about 6 to 12 carbon atoms. The aliphatic group is typically a saturated aliphatic group. Preferably, both R₈ and R₉ are aromatic or substituted aromatic groups, and the aromatic group may be a fused ring aromatic group such as naphthyl. Aromatic groups R₈ and R₉ may be joined together with other groups such as S.

Typical aromatic amines antioxidants have alkyl substituent groups of at least about 6 carbon atoms. Examples of aliphatic groups include hexyl, heptyl, octyl, nonyl, and decyl. Generally, the aliphatic groups will not contain more than about 14 carbon atoms. The general types of amine antioxidants useful in the present compositions include diphenylamines, phenyl naphthylamines, phenothiazines, imidodibenzyls and diphenyl phenylene diamines. Mixtures of two or more aromatic amines are also useful. Polymeric amine antioxidants can also be used. Particular examples of aromatic amine antioxidants useful in the present disclosure include: p,p′-dioctyldiphenylamine; t-octylphenyl-alpha-naphthylamine; phenyl-alpha-naphthylamine; and p-octylphenyl-alpha-naphthylamine.

Sulfur-containing antioxidants are also useful herein. In particular, one or more oil-soluble or oil-dispersible sulfur-containing antioxidant(s) can be used as an antioxidant additive. For example, sulfurized alkyl phenols and alkali or alkaline earth metal salts thereof also are useful antioxidants herein. Suitably, the lubricating oil composition(s) of the present disclosure may include the one or more sulfur-containing antioxidant(s) in an amount to provide the lubricating oil composition with from 0.02 to 0.2, preferably from 0.02 to 0.15, even more preferably 0.02 to 0.1, even more preferably 0.04 to 0.1, mass % sulfur based on the total mass of the lubricating oil composition. Optionally the oil-soluble or oil-dispersible sulfur-containing antioxidant(s) are selected from sulfurized C₄ to C₂₅ olefin(s), sulfurized aliphatic (C₇ to C₂₉) hydrocarbyl fatty acid ester(s), ashless sulfurized phenolic antioxidant(s), sulfur-containing organo-molybdenum compound(s), and combinations thereof. For further information, on sulfurized materials useful as antioxidants herein, please see U.S. Pat. No. 10,731,101 (col 15, ln 55 to col 22, ln 12).

Antioxidants useful herein include hindered phenols and/or arylamines. These antioxidants may be used individually by type or in combination with one another.

Typical antioxidants include: Irganox™ L67, Ethanox™ 4702, Lanxess Additin™ RC 7110; Ethanox™ 4782J; Irganox™ 1135, Irganox™ 5057, sulfurized lard oil and palm oil fatty acid methyl ester.

Antioxidant additives may be used in an amount of about 0.01 to 10 (alternately 0.01 to 5, alternately 0.01 to 3) wt %, alternately about 0.03 to 5 wt %, alternately 0.05 to less than 3 wt %, based upon the weight of the lubricating composition.

Compositions according to the present disclosure may contain an additive having a different enumerated function that also has secondary effects as an antioxidant (for example, phosphorus-containing antiwear agents (such as ZDDP) may also have antioxidant effects). These additives are not included as antioxidants for purposes of determining the amount of antioxidant in a lubricating oil composition or concentrate herein.

### F. Pour Point Depressants

Conventional pour point depressants (also known as lube oil flow improvers) may be added to the compositions of the present disclosure if desired. These pour point depressants may be added to lubricating compositions of the present disclosure to lower the minimum temperature at which the fluid will flow or can be poured. Examples of suitable pour point depressants include polymethacrylates, polyacrylates, polyarylamides, condensation products of haloparaffin waxes and aromatic compounds, vinyl carboxylate polymers, and terpolymers of dialkylfumarates, vinyl esters of fatty acids and allyl vinyl ethers. U.S. Pat. Nos. 1,815,022; 2,015,748; 2,191,498; 2,387,501; 2,655,479; 2,666,746; 2,721,877; 2,721,878; and 3,250,715 describe useful pour point depressants and/or the preparation thereof. Such additives may be used in an amount of about 0.01 to 5 wt %, preferably about 0.01 to 1.5 wt %, based upon the weight of the lubricating composition.

### G. Anti-Foam Agents

Anti-foam agents may advantageously be added to lubricant compositions described herein. These agents prevent or retard the formation of stable foams. Silicones and/organic polymers are typical anti-foam agents. For example, polysiloxanes, such as silicon oil or polydimethyl siloxane, provide anti-foam properties.

Anti-foam agents are commercially available and may be used in minor amounts such as 5 wt % or less, 3 wt % or less, 1 wt % or less, 0.1 wt % or less, such as from 5 to wt % to 0.1 ppm such as from 3 wt % to 0.5 ppm, such as from 1 wt % to 10 ppm.

For example, it may be that the lubricating oil composition comprises an anti-foam agent comprising polyalkyl siloxane, such as a polydialkyl siloxane, for example, wherein the alkyl is a C₁-C₁₀ alkyl group, e.g., a polydimethylsiloxane (PDMS), also known as a silicone oil. Alternately, the siloxane is a poly(R³)siloxane, wherein R³ is one or more same or different linear branched or cyclic hydrocarbyls, such as alkyls or aryls, typically having 1 to 20 carbon atoms. It may be that, for example, the lubricating oil composition comprises a polymeric siloxane compound according to Formula 1 below wherein R¹ and R² are independently methyl, ethyl, propyl, butyl, pentyl, hexyl, heptyl, octyl, nonyl or decyl, phenyl, naphthyl, alkyl substituted phenyl, or isomers thereof (such as methyl, phenyl) and n is from 2 to 1000, such as 50 to 450, alternately such as 40 to 100.

Additionally, or alternatively, it may be that the lubricating oil composition comprises an organo-modified siloxane (OMS), such as a siloxane modified with an organo group such as a polyether (e.g., ethylene-propyleneoxide copolymer), long chain hydrocarbyl (e.g., C₁₁-C₁₀₀ alkyl), or aryl (e.g., C₆-C₁₄ aryl). It may be that, for example, the lubricating oil composition comprises an organo-modified siloxane compound according to Formula 1, wherein n is from 2 to 2000, such as 50 to 450 (alternately such as 40 to 100), and wherein R¹ and R² are the same or different, optionally wherein each of R¹ and R² is, independently an organo group, such as an organo group selected from polyether (e.g., ethylene-propyleneoxide copolymer), long chain hydrocarbyl (e.g., C₁₁-C₁₀₀ alkyl), or aryl (e.g., C₆-C₁₄ aryl). Preferably, one of R¹ and R² is CH₃.

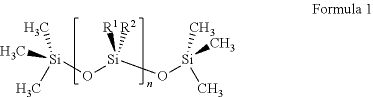

Based on the total weight of the lubricant composition, the siloxane according to Formula 1 is incorporated so as to provide about 0.1 to less than about 30 ppm Si, or about 0.1 to about 25 ppm Si, or about 0.1 to about 20 ppm Si, or about 0.1 to about 15 ppm Si, or about 0.1 to about 10 ppm Si. More preferably, it is in the range of about 3-10 ppm Si.

In embodiments, silicone anti-foam agents useful herein are available from Dow Corning Corporation and Union Carbide Corporation, such as Dow Corning FS-1265 (1000 centistokes), Dow Corning DC-200, and Union Carbide UC-L45. Silicone anti-foamants useful herein include polydimethylsiloxane, phenyl-methyl polysiloxane, linear, cyclic or branched siloxanes, silicone polymers and copolymers, and/organo-silicone copolymers. Also, a siloxane polyether copolymer Anti-foamant available from OSI Specialties, Inc. of Farmington Hills, Michigan and may be substituted or included. One such material is sold as SILWET-L-7220.

Acrylate polymer anti-foam agent can also be used herein. Typical acrylate anti-foamants include polyacrylate anti-foamant available from Monsanto Polymer Products Co. known as PC-1244. A preferred acrylate polymer anti-foam agent useful herein is PX™3841 (i.e., an alkyl acrylate polymer), commercially available from Dorf Ketl, also referred to as Mobilad™C402.

In embodiments, a combination of sililcone anti-foamant and acrylate anti-foamant can be used, such as at a weight ratio of the silicone anti-foamant to the acrylate anti-foamant of from about 5:1 to about 1:5, see, for example, US Patent Application Publication No. 2021/0189283.

### H. Viscosity Modifiers

Viscosity modifiers (also referred to as viscosity index improvers or viscosity improvers) can be included in the lubricating compositions described herein. Viscosity modifiers provide lubricants with high and low temperature operability. These additives impart shear stability at elevated temperatures and acceptable viscosity at low temperatures. Suitable viscosity modifiers include high molecular weight hydrocarbons, polyesters, and viscosity modifier dispersants that can function as both a viscosity modifier and a dispersant. Typical molecular weights of these polymers are between about 10,000 to 1,500,000 g/mol, more typically about 20,000 to 1,200,000 g/mol, and even more typically between about 50,000 and 1,000,000 g/mol.

Examples of suitable viscosity modifiers are linear or star-shaped polymers and copolymers of methacrylate, butadiene, olefins, or alkylated styrenes. Polyisobutylene is a commonly used viscosity modifier. Another suitable viscosity modifier is polymethacrylate (copolymers of various chain length alkyl methacrylates, for example), some formulations of which also serve as pour point depressants. Other suitable viscosity modifiers include copolymers of ethylene and propylene, hydrogenated block copolymers of styrene and isoprene, and polyacrylates (copolymers of various chain length acrylates, for example). Specific examples include styrene-isoprene or styrene-butadiene based polymers of 50,000 to 200,000 g/mol molecular weight.

Copolymers useful as viscosity modifiers include those commercially available from Chevron Oronite Company LLC under the trade designation “PARATONE™” (such as “PARATONE™ 8921,” “PARATONE™ 68231,” and “PARATONE™ 8941”); from Afton Chemical Corporation under the trade designation “HiTEC™” (such as HiTEC™ 5850B, and HiTEC™5777); and from The Lubrizol Corporation under the trade designation “Lubrizol™ 7067C”. Hydrogenated polyisoprene star polymers useful as viscosity modifiers herein include those commercially available from Infineum International Limited, e.g., under the trade designation “SV200™” and “SV600™”. Hydrogenated diene-styrene block copolymers useful as viscosity modifiers herein are commercially available from Infineum International Limited, e.g., under the trade designation “SV 50™”

Polymers useful as viscosity modifiers herein include polymethacrylate or polyacrylate polymers, such as linear polymethacrylate or polyacrylate polymers, such as those available from Evnoik Industries under the trade designation “Viscoplex™” (e.g., Viscoplex™ 6-954) or star polymers which are available from Lubrizol Corporation under the trade designation Asteric™ (e.g., Lubrizol™ 87708 and Lubrizol™ 87725).

Vinyl aromatic-containing polymers useful as viscosity modifiers herein may be derived from vinyl aromatic hydrocarbon monomers, such as styrenic monomers, such as styrene. Illustrative vinyl aromatic-containing copolymers useful herein may be represented by the following general formula: A-B wherein A is a polymeric block derived predominantly from vinyl aromatic hydrocarbon monomer (such as styrene), and B is a polymeric block derived predominantly from conjugated diene monomer (such as isoprene).

Vinyl aromatic-containing polymers useful as viscosity modifiers may have a Kinematic viscosity at 100° C. of 20 cSt or less, such as 15 cSt or less, such as 12 cSt or less, but may be diluted (such as in Group I, II, and/or III basestock) to higher Kinematic viscosities at 100° C., such as to 40 cSt or more, such as 100 cSt or more, such as 1000 cSt or more, such as 1000 to 2000 cSt.).

Typically, the viscosity modifiers may be used in an amount of about 0.01 to about 10 wt %, such as about 0.1 to about 7 wt %, such as 0.1 to about 4 wt %, such as about 0.2 to about 2 wt %, such as about 0.2 to about 1 wt %, and such as about 0.2 to about 0.5 wt %, based on the total weight of the formulated lubricant composition.

Viscosity modifiers are typically added as concentrates, in large amounts of diluent oil. The “as delivered” viscosity modifier typically contains from 20 wt % to 75 wt % of an active polymer for polymethacrylate or polyacrylate polymers, or from 8 wt % to 20 wt % of an active polymer for olefin copolymers, hydrogenated polyisoprene star polymers, or hydrogenated diene-styrene block copolymers, in the “as delivered” polymer concentrate.

### I. Dispersants

During engine operation, oil-insoluble oxidation byproducts are produced. Dispersants help keep these byproducts in solution, thus diminishing their deposition on metal surfaces. Dispersants used in the formulation of the lubricating compositions herein may be ashless or ash-forming in nature. Preferably, the dispersant is ashless. So called ashless dispersants are organic materials that form substantially no ash upon combustion. For example, non-metal-containing or borated metal-free dispersants are considered ashless. In contrast, metal-containing detergents tend to form ash upon combustion.

Dispersants useful herein typically contain a polar group attached to a relatively high molecular weight hydrocarbon chain. The polar group typically contains at least one element of nitrogen, oxygen, or phosphorus. Typical hydrocarbon chains contain 40 to 500, such as 50 to 400 carbon atoms.

### Dispersants of (Poly)Alkenylsuccinic Derivatives

A particularly useful class of dispersants includes the (poly)alkenylsuccinic derivatives, typically produced by the reaction of a long chain hydrocarbyl-substituted succinic compound, usually a hydrocarbyl-substituted succinic anhydride, with a polyhydroxy or polyamino compound. The long chain hydrocarbyl group constituting the oleophilic portion of the molecule which confers solubility in the oil, is often a polyisobutylene group (typically the long chain hydrocarbyl group, such as a polyisobutylene group, has an Mn of 400 to 3000 g/mol, such as 450 to 2500 g/mol). Many examples of this type of dispersant are well known commercially and in the literature. Exemplary US Patents describing such dispersants include U.S. Pat. Nos. 3,172,892; 3,2145,707; 3,219,666; 3,316,177; 3,341,542; 3,444,170; 3,454,607; 3,541,012; 3,630,904; 3,632,511; 3,787,374 and 4,234,435. Other types of dispersants are described in U.S. Pat. Nos. 3,036,003; 3,200,107; 3,254,025; 3,275,554; 3,438,757; 3,454,555; 3,565,804; 3,413,347; 3,697,574; 3,725,277; 3,725,480; 3,726,882; 4,454,059; 3,329,658; 3,449,250; 3,519,565; 3,666,730; 3,687,849; 3,702,300; 4,100,082; 5,705,458. A further description of dispersants useful herein may be found, for example, in European Patent Applications Nos. 0 471 071 and 0 451 380, to which reference is made for this purpose.

Hydrocarbyl-substituted succinic acid and hydrocarbyl-substituted succinic anhydride derivatives are useful dispersants. In particular, succinimide, succinate esters, or succinate ester amides prepared by the reaction of a hydrocarbon-substituted succinic acid or anhydride compound (typically having at least 25 carbon atoms, such as 28 to 400 carbon atoms, in the hydrocarbon substituent), with at least one equivalent of a polyhydroxy or polyamino compound (such as an alkylene amine) are particularly useful herein. Hydrocarbyl-substituted succinic acid and hydrocarbyl-substituted succinic anhydride derivatives may have a number average molecular weight of at least 400 g/mol, such as at least 900 g/mol, such as at least 1500 g/mol, such as from 400 to 4000 g/mol, such as from 800 to 3000, such as from 2000 to 2800 g/mol, such from about 2100 to 2500 g/mol, and such as from about 2200 to about 2400 g/mol.

Succinimides, which are particularly useful herein, are formed by the condensation reaction between: 1) hydrocarbyl-substituted succinic anhydrides, such as polyisobutylene succinic anhydride (PIBSA); and 2) polyamine (PAM). Examples of suitable polyamines include: polyhydrocarbyl polyamines, polyalkylene polyamines, hydroxy-substituted polyamines, polyoxyalkylene polyamines, and combinations thereof. Examples of polyamines include tetraethylene pentamine, pentaethylene hexamine, tetraethylenepentamine (TEPA), pentaethylenehaxamine (PEHA), N-phenyl-p-phenylenediamine (ADPA), and other polyamines having an average of 5, 6, 7, 8, or 9 nitrogen atoms per molecule. Mixtures where the average number of nitrogen atoms per polyamine molecule is greater than 7 are commonly called heavy polyamines or H-PAMs and may be commercially available under trade names such as HPA™ and HPA-X™ from DowChemical, E-100™ from Huntsman Chemical, et al. Examples of hydroxy-substituted polyamines include N-hydroxyalkyl-alkylene polyamines such as N-(2-hydroxyethyl)ethylene diamine, N-(2-hydroxyethyl)piperazine, and/or N-hydroxyalkylated alkylene diamines of the type described, for example, in U.S. Pat. No. 4,873,009. Examples of polyoxyalkylene polyamines include polyoxyethylene and/or polyoxypropylene diamines and triamines (as well as co-oligomers thereof) having an average Mn from about 200 to about 5000 g/mol. Products of this type are commercially available under the tradename Jeffamine™. Representative examples of useful succinimides are shown in U.S. Pat. Nos. 3,087,936; 3,172,892; 3,219,666; 3,272,746; 3,322,670; 3,652,616; 3,948,800; and 6,821,307; and CA Patent No. 1,094,044.

The dispersants may comprise one or more, optionally borated, higher molecular weight (Mn 1600 g/mol or more, such as 1800 to 3000 g/mol) succinimides and one or more, optionally borated, lower molecular weight (Mn less than 1600 g/mol) succinimides, where the higher molecular weight may be 1600 to 3000 g/mol, such as 1700 to 2800 g/mol, such as 1800 to 2500 g/mol, such as 1850 to 2300 g/mol; and the lower molecular weight may be 600 to less than 1600 g/mol, such as 650 to 1500 g/mol, such as 700 to 1400 g/mol, such as 800 to 1300 g/mol, such as 850 to 1200 g/mol such as 900 to 1150 g/mol, such as 900 to 1000 g/mol. The higher molecular weight succinimide dispersant may be present in the lubricating composition in an amount of from 0.5 to 10 wt %, or from 0.8 to 6 wt %, or from 1.0 to 5 wt %, or from 1.5 to 5 wt %, or from 1.5 to 4.0 wt %; and the lower molecular weight succinimides dispersant may be present in the lubricating composition in an amount of from 1 to 5 wt %, or from 1.5 to 4.8 wt %, or from 1.8 to 4.6 wt %, or from 1.9 to 4.6 wt %, or at 2 wt % or more, such as 2 to 5 wt %. The lower molecular weight succinimides may differ from the higher molecular weight succinimides, by 500 g/mol or more, such as by 750 g/mol or more, such as by 1000 g/mol or more, such as by 1200 g/mol or more, such as by 500 to 3000 g/mol, such as by 750 to 2000 g/mol, such as by 1000 to 1500 g/mol.

Succinate esters useful as dispersants include those formed by the condensation reaction between hydrocarbyl-substituted succinic anhydrides and alcohols or polyols. For example, the condensation product of a hydrocarbyl-substituted succinic anhydride and pentaerythritol is a useful dispersant.

Succinate ester amides useful herein are formed by a condensation reaction between hydrocarbyl-substituted succinic anhydrides and alkanol amines. Suitable alkanol amines include ethoxylated polyalkylpolyamines, propoxylated polyalkylpolyamines, and polyalkenylpolyamines such as polyethylene polyamines and/or propoxylated hexamethylenediamine. Representative examples are shown in U.S. Pat. No. 4,426,305.

Hydrocarbyl-substituted succinic anhydrides (such as PIBSA) esters of hydrocarbyl bridged aryloxy alcohols are also useful as dispersants herein. For information on such dispersants, please see U.S. Pat. No. 7,485,603, particularly, col 2, ln 65 to col 6, ln 22 and col 23, ln 40 to col 26, ln 46. In particular, PIBSA esters of methylene-bridged naphthyloxy ethanol (i.e., 2-hydroxyethyl-1-naphthol ether (or hydroxy-terminated ethylene oxide oligomer ether of naphthol) are useful herein.

The molecular weight of the hydrocarbyl-substituted succinic anhydrides used in the preceding paragraphs will typically range from 350 to 4000 g/mol, such as 400 to 3000 g/mol, such as 450 to 2800 g/mol, such as 800 to 2500 g/mol. The above (poly)alkenylsuccinic derivatives can be post-reacted with various reagents such as sulfur, oxygen, formaldehyde, carboxylic acids such as oleic acid.

The dispersants may be present in the lubricant in an amount 0.1 mass % to 20 mass % of the composition, such as 0.2 to 15 mass %, such as 0.25 to 10 mass %, such as 0.3 to 5 mass %, such as 1.0 mass % to 3.0 mass %, of the lubricating oil composition.

The above (poly)alkenylsuccinic derivatives, can also be post reacted with boron compounds such as boric acid, borate esters or highly borated dispersants, to form borated dispersants generally having from about 0.1 to about 5 moles of boron per mole of dispersant reaction product.

Dispersants useful herein include borated succinimides, including those derivatives from mono-succinimides, bis-succinimides, and/or mixtures of mono- and bis-succinimides, wherein the hydrocarbyl succinimide is derived from a hydrocarbylene group such as polyisobutylene having an Mn of from about 300 to about 5000 g/mol, or from about 500 to about 3000 g/mol, or about 1000 to about 2000 g/mol, or a mixture of such hydrocarbylene groups, often with high terminal vinylic groups.

The boron-containing dispersant may be present at 0.01 wt % to 20 wt %, or 0.1 wt % to 15 wt %, or 0.1 wt % to 10 wt %, or 0.5 wt % to 8 wt %, or 1.0 wt % to 6.5 wt %, or 0.5 wt % to 2.2 wt % of the lubricating composition.

The boron-containing dispersant may be present in an amount to deliver boron to the composition at 15 ppm to 2000 ppm, or 25 ppm to 1000 ppm, or 40 ppm to 600 ppm, or 80 ppm to 350 ppm.

The borated dispersant may be used in combination with non-borated dispersant and may be the same or different compound as the non-borated dispersant. In one embodiment, the lubricating composition may include one or more boron-containing dispersants and one or more non-borated dispersants, wherein the total amount of dispersant may be 0.01 wt % to 20 wt %, or 0.1 wt % to 15 wt %, or 0.1 wt % to 10 wt %, or 0.5 wt % to 8 wt %, or 1.0 wt % to 6.5 wt %, or 0.5 wt % to 2.2 wt % of the lubricating composition and wherein the ratio of borated dispersant to non-boroated dispersant may be 1:10 to 10:1 (weight:weight) or 1:5 to 3:1 or 1:3 to 2:1.

The dispersant may comprise one or more borated or unborated poly(alkenyl)succinimides, where the polyalkyenyl is derived from polyisobutylene and the imide is derived from a polyamine (“PIBSA-PAM”).

The dispersant may comprise one or more PIBSA-PAMs, where the PIB is derived from polyisobutylene having an Mn of from 600 to 5000, such as from 700 to 4000, such as from 800 to 3000, such as from 900 to 2500 g/mol and the polyamine is derived from hydrocarbyl-substituted polyamines, such as tetraethylene pentamine, pentaethylene hexamine, tetraethylenepentamine (TEPA), pentaethylenehaxamine (PEHA), N-phenyl-p-phenylenediamine (ADPA), and other polyamines having an average of 5, 6, 7, 8, or 9 nitrogen atoms per molecule). The dispersant may be borated, typically at levels of up to 4 mass % such as from 1 to 3 mass %. The dispersant may comprise one or more borated and one or more non-borated PIBSA-PAM's. The dispersant may comprise one or more borated PIBSA-PAM's derived from a PIB having an Mn of 700 to 1800 g/mol (such as 800 to 1500 g/mol) and one or more non-borated PIBSA-PAM's derived from a PIB having an Mn of more than 1800 to 5000 g/mol (such as 2000 to 3000 g/mol). The dispersant may comprise one or more non-borated PIBSA-PAM's derived from a PIB having an Mn of 700 to 1800 g/mol (such as 800 to 1500 g/mol) and one or more borated PIBSA-PAM's derived from a PIB having an Mn of more than 1800 to 5000 g/mol (such as 2000 to 3000 g/mol).

The dispersant may comprise PIBSA derived from a PIB having an Mn of 700 to 5000 g/mol (such as 800 to 3000 g/mol) and one or more borated or non-borated PIBSA-PAM's derived from a PIB having an Mn of 700 to 5000 g/mol.

The dispersant may comprise PIBSA derived from a PIB having an Mn of 700 to 5000 g/mol (such as 800 to 3000 g/mol) and one or more borated PIBSA-PAM's derived from a PIB having an Mn of 700 to 1800 g/mol (such as 800 to 1500 g/mol) and one or more non-borated PIBSA-PAM's derived from a PIB having an Mn of more than 1800 to 5000 g/mol (such as 2000 to 3000 g/mol). The dispersant may comprise PIBSA derived from a PIB having an Mn of 700 to 5000 g/mol (such as 800 to 3000 g/mol) one or more non-borated PIBSA-PAM's derived from a PIB having an Mn of 700 to 1800 g/mol (such as 800 to 1500 g/mol) and one or more borated PIBSA-PAM's derived from a PIB having an Mn of more than 1800 to 5000 g/mol (such as 2000 to 3000 g/mol).

The dispersant may comprise one or more borated or non-borated PIBSA-PAM's and one or more PIBSA-esters of hydrocarbyl bridged aryloxy alcohols.

The dispersant may comprise one or more borated and one or more non-borated PIBSA-PAM's.

The dispersant may comprise one or more, optionally borated, higher molecular weight (Mn 1600 g/mol or more, such as 1800 to 3000 g/mol) PIBSA-PAM's and one or more, optionally borated, lower molecular weight (Mn less than 1600 g/mol) PIBSA-PAM's, where the higher molecular weight may be 1600 to 3000 g/mol, such as 1700 to 2800 g/mol, such as 1800 to 2500 g/mol, such as 1850 to 2300 g/mol; and the lower molecular weight may be 600 to less than 1600 g/mol, such as 650 to 1500 g/mol, such as 700 to 1400 g/mol, such as 800 to 1300 g/mol, such as 850 to 1200 g/mol, such as 900 to 11500 g/mol, such as 900 to 100 g/mol. The higher molecular weight PIBSA-PAM dispersant may be present in the lubricating composition in an amount of from 0.5 to 10 wt %, or from 0.8 to 6 wt %, or from 1.0 to 5 wt %, or from 1.5 to 5 wt % or from 1.5 to 4.0 wt %; and the lower molecular weight PIBSA-PAM dispersant may be present in the lubricating composition in an amount of from 1 to 5 wt %, or from 1.5 to 4.8 wt %, or from 1.8 to 4.6 wt %, or from 1.9 to 4.6 wt %, or at 2 wt % or more, such as 2 to 5 wt %.

### Dispersants of Mannich Bases

Mannich base dispersants useful herein are typically made from the reaction of an amine component, a hydroxy aromatic compound (substituted or unsubstituted, such as alkyl substituted), such as alkylphenols, and an aldehyde, such as formaldehyde. See U.S. Pat. Nos. 4,767,551 and 10,899,986. Process aids and catalysts, such as oleic acid and sulfonic acids, can also be part of the reaction mixture. Representative examples are shown in U.S. Pat. Nos. 3,697,574; 3,703,536; 3,704,308; 3,751,365; 3,756,953; 3,798,165; 3,803,039; 4,231,759; 9,938,479; 7,491,248; and 10,899,986, and PCT Publication No. WO 01/42399.

### Dispersants of Polymethacrylate or Polyacrylate Derivatives

Polymethacrylate or polyacrylate derivatives are another class of dispersants useful herein. These dispersants are typically prepared by reacting a nitrogen-containing monomer and a methacrylic or acrylic acid esters containing 5-25 carbon atoms in the ester group. Representative examples are shown in U.S. Pat. Nos. 2,100,993, and 6,323,164. Polymethacrylate and polyacrylate dispersants are typically lower molecular weights.

The lubricating composition of the disclosure typically comprises dispersant at 0.1 mass % to 20 mass % of the composition, such as 0.2 to 15 mass %, such as 0.25 to 10 mass %, such as 0.3 to 5 mass %, such as 2.0 mass % to 4.0 mass % of the lubricating oil composition. Alternately the dispersant may be present at 0.1 wt % to 5 wt %, or 0.01 wt % to 4 wt % of the lubricating composition.

For further information on dispersants useful herein, please see U.S. Pat. No. 10,829,712, col 13, ln 36 to col 16, ln 67 and U.S. Pat. No. 7,485,603, col 2, ln 65 to col 6, ln 22, col 8, ln 25 to col 14, ln 53, and col 23, ln 40 to col 26, ln 46.

Compositions according to the present disclosure may contain an additive having a different enumerated function that also has secondary effects as a dispersant (for example, Component B Functionalized Polymer described above, may also have dispersant effects). These additives are not included as dispersants for purposes of determining the amount of dispersant in a lubricating oil composition or concentrate herein.

### J. Corrosion Inhibitors/Anti-Rust Agents

Corrosion inhibitors may be used to reduce the corrosion of metals and are often alternatively referred to as metal deactivators or metal passivators. Some corrosion inhibitors may alternatively be characterized as antioxidants.

Suitable corrosion inhibitors may include nitrogen and/or sulfur-containing heterocyclic compounds such as triazoles (e.g., benzotriazoles), substituted thiadiazoles, imidazoles, thiazoles, tetrazoles, hydroxyquinolines, oxazolines, imidazolines, thiophenes, indoles, indazoles, quinolines, benzoxazines, dithiols, oxazoles, oxatriazoles, pyridines, piperazines, triazines and derivatives of any one or more thereof. A particular corrosion inhibitor is a benzotriazole represented by the structure:

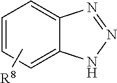

wherein R⁸ is absent (hydrogen) or is a C₁ to C₂₀ hydrocarbyl or substituted hydrocarbyl group which may be linear or branched, saturated or unsaturated. It may contain ring structures that are alkyl or aromatic in nature and/or contain heteroatoms such as N, O, or S. Examples of suitable compounds may include benzotriazole, alkyl-substituted benzotriazoles (e.g., tolyltriazole, ethylbenzotriazole, hexylbenzotriazole, octylbenzotriazole, etc.), aryl substituted benzotriazole, alkylaryl- or arylalkyl-substituted benzotriazoles, and the like, as well as combinations thereof. For instance, the triazole may comprise or be a benzotriazole and/or an alkylbenzotriazole in which the alkyl group contains from 1 to about 20 carbon atoms or from 1 to about 8 carbon atoms. Non-limiting examples of such corrosion inhibitors may comprise or be benzotriazole, tolyltriazole, and/or optionally, substituted benzotriazoles such as Irgamet™ 39, which is commercially available from BASF of Ludwigshafen, Germany. A preferred corrosion inhibitor may comprise or be benzotriazole and/or tolyltriazole.

Additionally, or alternatively, the corrosion inhibitor may include one or more substituted thiadiazoles represented by the structure:

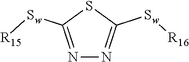

wherein R₁₅ and R₁₆ are independently hydrogen or a hydrocarbon group, which group may be aliphatic or aromatic, including cyclic, alicyclic, aralkyl, aryl and alkaryl, and wherein each w is independently 1, 2, 3, 4, 5, or 6 (preferably 2, 3, or 4, such as 2). These substituted thiadiazoles are derived from the 2,5-dimercapto-1,3,4-thiadiazole (DMTD) molecule. Many derivatives of DMTD have been described in the art, and any such compounds may be included in the fluid used in the present disclosure. For example, U.S. Pat. Nos. 2,719,125; 2,719,126; and 3,087,937; describe the preparation of various 2, 5-bis-(hydrocarbon dithio)-1,3,4-thiadiazoles.

Further, additionally or alternatively, the corrosion inhibitor may include one or more other derivatives of DMTD, such as a carboxylic ester in which R₁₅ and R₁₆ may be joined to the sulfide sulfur atom through a carbonyl group. Preparation of these thioester-containing DMTD derivatives is described, for example, in U.S. Pat. No. 2,760,933. DMTD derivatives produced by condensation of DMTD with alpha-halogenated aliphatic carboxylic acids having at least 10 carbon atoms are described, for example, in U.S. Pat. No. 2,836,564. This process produces DMTD derivatives wherein R₁₅ and R₁₆ are HOOC—CH(R₁₉)—(R₁₉ being a hydrocarbyl group). DMTD derivatives further produced by amidation or esterification of these terminal carboxylic acid groups may also be useful.

The preparation of 2-hydrocarbyldithio-5-mercapto-1,3,4-thiadiazoles is described, for example, in U.S. Pat. No. 3,663,561.

A class of DMTD derivatives may include mixtures of a 2-hydrocarbyldithio-5-mercapto-1,3,4-thiadiazole and a 2,5-bis-hydrocarbyldithio-1,3,4-thiadiazole. Such mixtures may be sold under the tradename HiTEC™ 4313 and are commercially available from Afton Chemical Company.

The preparation of 2-hydrocarbyldithio-5-mercapto-1,3,4-thiadiazoles is described, for example, in U.S. Pat. No. 3,663,561.

A class of DMTD derivatives may include mixtures of a 2-hydrocarbyldithio-5-mercapto-1,3,4-thiadiazole and a 2,5-bis-hydrocarbyldithio-1,3,4-thiadiazole. Such mixtures may be sold under the tradename HiTEC™ 4313 and are commercially available from Afton Chemical Company.

Still further, additionally or alternatively, the corrosion inhibitor may include a trifunctional borate having the structure, B(OR₄₆)₃, in which each R₄₆ may be the same or different. As the borate may typically be desirably compatible with the non-aqueous medium of the composition, each R₄₆ may, in particular, comprise or be a hydrocarbyl C₁-C₈ moiety. For compositions in which the non-aqueous medium comprises or is a lubricating oil basestock, for example, better compatibility can typically be achieved when the hydrocarbyl moieties are each at least C₄. Non-limiting examples of such corrosion inhibitors thus include, but are not limited to, triethylborate, tripropylborates such as triisopropylborate, tributylborates such as tri-tert-butylborate, tripentylborates, trihexylborates, trioctylborates such as tri-(2-ethylhexyl)borate, monohexyl dibutylborate, and the like, as well as combinations thereof.

When used, a corrosion inhibitor may comprise a substituted thiadiazole, a substituted benzotriazole, a substituted triazole, a trisubstituted borate, or a combination thereof.

When desired, corrosion inhibitors can be used in any effective amount, but, when used, may typically be used in amounts from about 0.001 wt % to 5.0 wt %, based on the weight of the composition, e.g., from 0.005 wt % to 3.0 wt % or from 0.01 wt % to 1.0 wt %. Alternately, such additives may be used in an amount of about 0.01 to 5 wt %, preferably about 0.01 to 1.5 wt %, based upon the weight of the lubricating composition.

In some embodiments, 3,4-oxypyridinone-containing compositions may contain substantially no (e.g., 0, or less than 0.001 wt %, 0.0005 wt % or less, not intentionally added, and/or absolutely no) triazoles, benzotriazoles, substituted thiadiazoles, imidazoles, thiazoles, tetrazoles, hydroxyquinolines, oxazolines, imidazolines, thiophenes, indoles, indazoles, quinolines, benzoxazines, dithiols, oxazoles, oxatriazoles, pyridines, piperazines, triazines, derivatives thereof, combinations thereof, or all corrosion inhibitors.

Compositions according to the present disclosure may contain an additive having a different enumerated function that also has secondary effects as a corrosion inhibitor (for example, Component B Functionalized Polymer described above, may also have corrosion inhibitor effects). These additives are not included as corrosion inhibitor for purposes of determining the amount of corrosion inhibitor in a lubricating oil composition or concentrate herein.

### K. Antiwear Agents

The lubricating oil composition of the present disclosure can contain one or more antiwear agents that can reduce friction and excessive wear. Any antiwear agent known by a person of ordinary skill in the art may be used in the lubricating oil composition. Non-limiting examples of suitable antiwear agents include zinc dithiophosphate, metal (e.g., Pb, Sb, Mo, and the like) salts of dithiophosphates, metal (e.g., Zn, Pb, Sb, Mo, and the like) salts of dithiocarbamates, metal (e.g., Zn, Pb, Sb, and the like) salts of fatty acids, boron compounds, phosphate esters, phosphite esters, amine salts of phosphoric acid esters or thiophosphoric acid esters, reaction products of dicyclopentadiene and thiophosphoric acids and combinations thereof. The amount of the antiwear agent may vary from about 0.01 wt % to about 5 wt %, from about 0.05 wt % to about 3 wt %, or from about 0.1 wt % to about 1 wt %, based on the total weight of the lubricating oil composition.

In embodiments, the antiwear agent is or comprises a dihydrocarbyl dithiophosphate metal salt, such as zinc dialkyl dithiophosphate compounds. The metal of the dihydrocarbyl dithiophosphate metal salt may be an alkali or alkaline earth metal, or aluminum, lead, tin, molybdenum, manganese, nickel, or copper. In some embodiments, the metal is zinc. In other embodiments, the alkyl group of the dihydrocarbyl dithiophosphate metal salt has from about 3 to about 22 carbon atoms, from about 3 to about 18 carbon atoms, from about 3 to about 12 carbon atoms, or from about 3 to about 8 carbon atoms. In further embodiments, the alkyl group is linear or branched.

Useful antiwear agents also include substituted or unsubstituted thiophosphoric acids, and salts thereof include zinc-containing compounds such as zinc dithiophosphate compounds selected from zinc dialkyl-, diaryl- and/or alkylaryl-dithiophosphates.

A metal alkylthiophosphate and more particularly a metal dialkyl dithio phosphate in which the metal constituent is zinc, or zinc dialkyl dithio phosphate (ZDDP) can be a useful component of the lubricating compositions of this disclosure. ZDDP can be derived from primary alcohols, secondary alcohols or mixtures thereof. ZDDP compounds generally are of the formula Zn[SP(S)(OR₁)(OR₂)]₂ where R₁ and R₂ are C₁-C₁₈ alkyl groups, preferably C₂-C₁₂ alkyl groups. These alkyl groups may be straight chain or branched. Alcohols used in the ZDDP can be 2-propanol, butanol, secondary butanol, pentanols, hexanols such as 4-methyl-2-pentanol, n-hexanol, n-octanol, 2-ethyl hexanol, alkylated phenols, and the like. Mixtures of secondary alcohols or of primary and secondary alcohol can be used. Alkyl aryl groups may also be used. Useful zinc dithiophosphates include secondary zinc dithiophosphates such as those available from The Lubrizol Corporation under the trade designations “LZ 677A”, “LZ 1095” and “LZ 1371”, from Chevron Oronite under the trade designation “OLOA 262” and from Afton Chemical under the trade designation “HiTEC™ 7169”.

In embodiments, the zinc compound can be a zinc dithiocarbamate complex, such as the zinc dithiocarbamates represented by the formula:

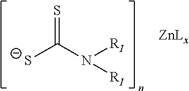

where each R₁ is independently a linear, cyclic, or branched, saturated or unsaturated, aliphatic hydrocarbon moiety having from 1 to about 10 carbon atoms, n is 0, 1, or 2, L is a ligand that saturates the coordination sphere of zinc, and x is 0, 1, 2, 3, or 4. In certain embodiments, the ligand, L, is selected from the group consisting of water, hydroxide, ammonia, amino, amido, alkylthiolate, halide, and combinations thereof.

The antiwear additives, such as ZDDP and/or the zinc carbamates, are typically used in amounts of from about 0.4 wt % to about 1.2 wt %, preferably from about 0.5 wt % to about 1.0 wt %, and more preferably from about 0.6 wt % to about 0.8 wt %, based on the total weight of the lubricating composition, although more or less can often be used advantageously. Preferably, the antiwear additive is ZDDP, preferably a secondary ZDDP, and is present in an amount of from about 0.6 to 1.0 wt % of the total weight of the lubricating composition.

Antiwear additives useful herein also include boron-containing compounds, such as borate esters, borated fatty amines, borated epoxides, alkali metal (or mixed alkali metal or alkaline earth metal) borates and borated overbased metal salts.

Compositions according to the present disclosure may contain an additive having a different enumerated function that also has secondary effects as an antiwear agent (for example, Component B Functionalized Polymer described above, may also have antiwear effects). These additives are not included as antiwear agents for purposes of determining the amount of antiwear agents in a lubricating oil composition or concentrate herein.

### L. Demulsifiers

Demulsifiers useful herein include those described in U.S. Pat. No. 10,829,712 (col 20, ln 34-40). Typically, a small amount of a demulsifying component may be used herein. A preferred demulsifying component is described in European Patent No. 330 522. It is obtained by reacting an alkylene oxide with an adduct obtained by reacting a bis-epoxide with a polyhydric alcohol. Such additives may be used in an amount of about 0.001 to 5 wt %, preferably about 0.01 to 2 wt %.

### M. Seal Compatibility Agents

Other optional additives include seal compatibility agents such as organic phosphates, aromatic esters, aromatic hydrocarbons, esters (butylbenzyl phthalate, for example), and polybutenyl succinic anhydride. Such additives may be used in an amount of about 0.001 to 5 wt %, preferably about 0.01 to 2 wt %. In embodiments the seal compatibility agents are sea swell agents, such as PIBSA (polyisobutenyl succinic anhydride).

### N. Extreme Pressure Agents

The lubricating oil composition of the present disclosure can contain one or more extreme pressure agents that can prevent sliding metal surfaces from seizing under conditions of extreme pressure. Any extreme pressure agent known by a person of ordinary skill in the art may be used in the lubricating oil composition. Generally, the extreme pressure agent is a compound that can combine chemically with a metal to form a surface film that prevents the welding of asperities in opposing metal surfaces under high loads. Non-limiting examples of suitable extreme pressure agents include sulfurized animal or vegetable fats or oils, sulfurized animal or vegetable fatty acid esters, fully or partially esterified esters of trivalent or pentavalent acids of phosphorus, sulfurized olefins, dihydrocarbyl polysulfides, sulfurized Diels-Alder adducts, sulfurized dicyclopentadiene, sulfurized or co-sulfurized mixtures of fatty acid esters and monounsaturated olefins, co-sulfurized blends of fatty acid, fatty acid ester and alpha-olefin, functionally substituted dihydrocarbyl polysulfides, thia-aldehydes, thia-ketones, epithio compounds, sulfur-containing acetal derivatives, co-sulfurized blends of terpene and acyclic olefins, and poly sulfide olefin products, amine salts of phosphoric acid esters or thiophosphoric acid esters, and combinations thereof. The amount of the extreme pressure agent may vary from about 0.01 wt % to about 5 wt %, from about 0.05 wt % to about 3 wt %, or from about 0.1 wt % to about 1 wt %, based on the total weight of the lubricating oil composition.

### O. Non-Basestock Unsaturated Hydrocarbons

The lubricating oil composition of the present disclosure can contain one or more unsaturated hydrocarbons. These unsaturated hydrocarbons are distinct from any baseoils (lubricating oil basestocks of Group I, II, III, IV and/or V) and/or viscosity modifiers that may be present in the compositions and always have at least one (and typically only one, in the case of linear alpha-olefins, or LAOs) unsaturation per molecule. Without being bound by theory, the unsaturation(s) may provide an antioxidation functionality and/or a sulfur-trapping functionality that may supplement and/or replace one or more antioxidant additives and/or one or more corrosion inhibitor additives, but unsaturated hydrocarbons (LAOs) will typically not provide the only antioxidant nor the only corrosion inhibition functionality in lubrication oil compositions. Non-limiting examples of unsaturated hydrocarbons can include one or more unsaturated C₁₂-C₆₀ hydrocarbons (such as C₁₂-C₄₈ hydrocarbons, C₁₂-C₃₆ hydrocarbons, C₁₂-C₃₀ hydrocarbons, or C₁₂-C₂₄ hydrocarbons). When only one unsaturation is present, the unsaturated hydrocarbons may be termed linear alpha-olefins (LAOs). Other non-limiting examples of unsaturated hydrocarbons can include oligomers/polymers of polyisobutylenes that have retained (or been post-polymerization modified to exhibit) a (near-) terminal unsaturation, and/or blends thereof. When present, unsaturated hydrocarbons (LAOs) may be present from 0.01 to 5 wt % (in particular, 0.1 to 3 mass %, alternately 0.1 to 1.5 mass %), based on total weight of the lubricating oil composition.

When lubricating oil compositions contain one or more of the additives discussed above, the additive(s) are typically blended into the composition in an amount sufficient for it to perform its intended function. Typical amounts of such additives useful in the present disclosure, especially for use in crankcase lubricants, are shown in the Table below.

It is noted that many of the additives are shipped from the additive manufacturer as a concentrate, containing one or more additives together, with a certain amount of base oil or other diluents. Accordingly, the weight amounts in the table below, as well as other amounts mentioned herein, are directed to the amount of active ingredient (that is the non-diluent portion of the ingredient). The weight percent (mass %) indicated below is based on the total weight of the lubricating oil composition.

### Typical Amounts of Optional Lubricating Oil Components

| Additive Formulations | A (mass % a.i.) | B (mass % a.i.) | C (mass % a.i.) |
|---|---|---|---|
| Dispersant | 0.1-20 | 0.1-20 | 1-8 |
| Detergents | 0.1-20 | 0.1-20 | 0.2-9 |
| Corrosion Inhibitor Anti-rust/ | 0-7 | 0.05-5 | 0.1-1.5 |
| extreme pressure agents | | | |
| Antioxidant | 0.01-10 | 0.1-5 | 0.1-4 |
| Pour Point Depressant | | 0-5 | 0.01-1.5 |
| Anti-foaming Agent | | 0-5 | 0.001-0.15 |
| Functionalized Polymer | 0.01-10 | 0.1-5 | 0.1-2 |
| Friction Modifier | | 1. | 0.5 |
| Antiwear Agent | 0.01-10 | 0.1-5 | 0.1-3 |
| Viscosity Modifier | | 0.01-10 | 0.25-3 |
| Seal Swell Agents | | 0-5 | 0-2 |
| Extreme Pressure Agents | | 0-5 | 0-3 |
| Unsaturated Hydrocarbons (LAOs) | | 0-5 | 0-3 |
| Basestock | Balance (such | Balance | Balance |
| | as 50 to 95%) | | |

The foregoing additives are typically commercially available materials. These additives may be added independently, but are usually pre-combined in packages, which can be obtained from suppliers of lubricant oil additives. Additive packages with a variety of ingredients, proportions and characteristics are available and selection of the appropriate package will take the use of the ultimate composition into account.

### Fuels

This disclosure also relates to a method of lubricating an automotive internal combustion engine during operation of the engine comprising:
(i) providing to a crankcase of the automotive internal combustion engine an automotive crankcase the lubricating composition of described herein;
(ii) providing a hydrocarbon fuel in the automotive internal combustion engine; and
(iii) combusting the fuel in the automotive internal combustion engine, such as a spark-ignited or compression-ignited two- or four-stroke reciprocating engines such as a diesel engine or passenger car engine (such as a spark-ignited combustion engine).

This disclosure also relates to a fuel composition comprising the lubricating oil compositions described herein and a hydrocarbon fuel, wherein the fuel may be derived from petroleum and/or biological sources (“biofuel” or “renewable fuel”). In embodiments, the fuel comprises from 0.1 to 100 mass % renewable fuel, alternately from 1 to 75 mass % renewable fuel, alternately from 5 to 50 mass % renewable fuel, based upon the total mass of the from 1 to 50 mass % renewable fuel and the petroleum derived fuel.

The renewable fuel component is typically produced from vegetable oil (such as palm oil, rapeseed oil, soybean oil, jatropha oil), microbial oil (such as algae oil), animal fats (such as cooking oil, animal fat, and/or fish fat) and/or biogas. Renewable fuel refers to biofuel produced from biological resources formed through contemporary biological processes. In an embodiment, the renewable fuel component is produced by means of a hydrotreatment process. Hydrotreatment involves various reactions where molecular hydrogen reacts with other components, or the components undergo molecular conversions in the presence of molecular hydrogen and a solid catalyst. The reactions include, but are not limited to, hydrogenation, hydrodeoxygenation, hydrodesulfurization, hydrodenitrification, hydrodemetallization, hydrocracking, and isomerization. The renewable fuel component may have different distillation ranges, which provide the desired properties to the component, depending on the intended use.

### Uses

The lubricating compositions of the disclosure may be used to lubricate mechanical engine components, particularly in internal combustion engines, e.g., spark-ignited or compression-ignited, two- or four-stroke reciprocating engines, by adding the lubricant thereto. Typically, they are crankcase lubricants, such as passenger car motor oils or heavy-duty diesel engine lubricants.

In particular, the lubricating compositions of the present disclosure are suitably used in the lubrication of the crankcase of a compression-ignited, internal combustion engine, such as a heavy-duty diesel engine.

In particular, the lubricating compositions of the present disclosure are suitably used in the lubrication of the crankcase of a spark-ignited turbo charged internal combustion engine.

In embodiments, the lubricating oils of this disclosure are used in spark-assisted high compression internal combustion engines and, when used in high compression spark ignition internal combustion engines the lubricating oil compositions of this disclosure are useful in lubricating high compression spark ignition engines.

In embodiments, the lubricating compositions of the present disclosure are suitably used in the lubrication of the crankcase of an engine for a heavy-duty diesel vehicle (i.e., a heavy-duty diesel vehicle having a gross vehicle weight rating of 10,000 pounds or more.)

In embodiments, the lubricating compositions of the present disclosure are suitably used in the lubrication of the crankcase of a passenger car diesel engine.

In particular, lubricating oil formulations of this disclosure are particularly useful in compression-ignited internal combustion engines, i.e., heavy-duty diesel engines, employing low viscosity oils, such as API FA-4 and future oil categories, in which wear protection of the valve train becomes challenging.

### Additional Embodiments/EP Clauses

1. A copolymer comprising one or more of the following:
I. (a) 10.0 to 20.0 wt % of amine-derivatized alpha-methyl styrene (ADAMS) repeat units according to structure (I):

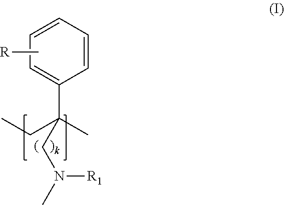

wherein: k is an integer from 1 to 3; R₁ a hydrogen or a benzyl group, R is hydrogen, a phenyl ring co-attached at two neighboring ring carbon positions with the phenyl ring shown so as to form a naphthalene assembly, a phenyl group attached at a single carbon of the phenyl ring shown, a C₁-C₄ hydrocarbyl group, a C₁-C₆ hydrocarbyl group containing 1 to 4 additional heteroatoms selected from the group consisting of O, N, S, P, Se, and combinations thereof, and (b) 80.0 to 90.0 wt % of repeat units corresponding to the reacted form of isoprene, and wherein the peak-average molecular weight of the copolymer is between 45.0 to 65.0 kDa;
II. (a) 5.0 to 10.0 wt % of amine-derivatized alpha-methyl styrene (ADAMS) repeat units according to structure (II):

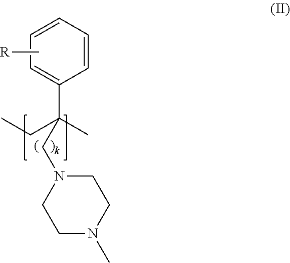

wherein: k is an integer from 1 to 3; R is hydrogen, a phenyl ring co-attached at two neighboring ring carbon positions with the phenyl ring shown so as to form a naphthalene assembly, a phenyl group attached at a single carbon of the phenyl ring shown, a C₁-C₄ hydrocarbyl group, a C₁-C₆ hydrocarbyl group containing 1 to 4 additional heteroatoms selected from the group consisting of O, N, S, P, Se, and combinations thereof, and (b) the remaining 90.0 to 95.0 wt % of repeat units corresponding to the reacted form of isoprene, and wherein, the peak-average molecular weight of the copolymer is between 24.0 to 42.0 kDa;
III. (a) 4.0 to 6.0 wt % of amine-derivatized alpha-methyl styrene (ADAMS) repeat units according to structure (III):

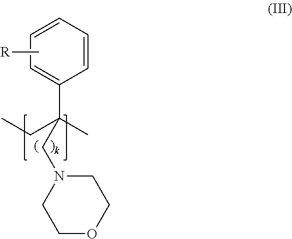

wherein: k is an integer from 1 to 3; R is hydrogen, a phenyl ring co-attached at two neighboring ring carbon positions with the phenyl ring shown so as to form a naphthalene assembly, a phenyl group attached at a single carbon of the phenyl ring shown, a C1-C4 hydrocarbyl group, a C1-C6 hydrocarbyl group containing 1 to 4 additional heteroatoms selected from the group consisting of O, N, S, P, Se, and combinations thereof, and (b) the remaining 94.0 to 96.0 wt % of repeat units corresponding to the reacted form of isoprene, and wherein, the peak-average molecular weight of the copolymer is between 36.0 to 46.0 kDa;
IV. (a) one or more amine-derivatized alpha-methyl styrene (ADAMS) repeat units according to structure (IV):

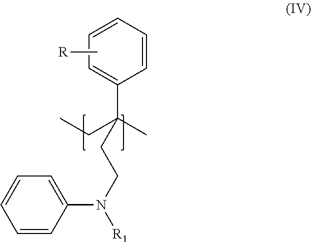

wherein: k is an integer from 1 to 3; R₁ a hydrogen or a benzyl group, R is hydrogen, a phenyl ring co-attached at two neighboring ring carbon positions with the phenyl ring shown so as to form a naphthalene assembly, a phenyl group attached at a single carbon of the phenyl ring shown, a C1-C4 hydrocarbyl group, a C1-C6 hydrocarbyl group containing 1 to 4 additional heteroatoms selected from the group consisting of O, N, S, P, Se, and combinations thereof, and (b) the remaining repeat units corresponding to the reacted form of isoprene.

2. The copolymer of clause 1, wherein the copolymer is partially or substantially hydrogenated.

3. The copolymer of clause 1 or 2, wherein k=2.

4. The copolymer of clauses 1-3, further comprising an alkyl residue from a monofunctional initiator selected from the group consisting of alkyl lithium, alkyl sodium, alkyl potassium and combinations thereof, and present at one or more termini of a polymer backbone.

5. The copolymer of clause 4, wherein the alkyl residues from the monofunctional initiator comprise methyl, ethyl, n-propyl, isopropyl, n-butyl, iso-butyl, sec-butyl, tert-butyl, n-amyl, iso-amyl, sec-amyl, tert-amyl, hexyl groups, or combinations thereof.

6. The copolymer of clauses 1-5, wherein one or more polymer blocks of the copolymer form a distributed polymer architecture, a diblock, a triblock, a tetrablock, a pentablock, a hexablock, a star polymer architecture, or combinations thereof.

7. A lubricating oil composition comprising or resulting form the admixing of: (i) at least 50 wt % of one or more base oils, based upon the weight of the lubricating oil composition; (ii) one or more dispersants; (iii) one or more detergents; and (iv) one or more copolymers of clauses I-6.

8. The lubricating oil composition of clause 7, wherein the composition has an SAE viscosity grade of 20 W-X, 15 W-X, 10 W-X, 5 W-X, or OW-X, where X represents any one of 8, 12, 16, 20, 30, 40, or 50.

9. The lubricating oil composition of clauses 7-8, comprising or resulting from the admixing of: (i) from 50 to 99 mass % of the one or more of the base oils, based upon the weight of the lubricating oil composition; (ii) from 0.01 to 20 wt % based on total weight of the lubricating oil composition, of the one or more dispersants; (iii) from 0.10 to 20 mass %, based upon the weight of the lubricating oil composition, of the one or more detergents; and (iv) from 0.10 to 20 mass %, based upon the weight of the lubricating oil composition, of the one or more copolymers.

10. The lubricating oil composition of clauses 7-9, further comprising one, two, three, four, five, six or more of additional additives selected from the group consisting of: friction modifiers; antioxidants; pour point depressants; anti-foam agents; viscosity modifiers; corrosion inhibitors and/or anti-rust agents; and antiwear agents.

11. The lubricating oil composition of clauses 7-10, further comprising one, two, three, four, five, six, or more of: A) from 0.01 to 5 wt %, based on total weight of the lubricating oil composition, of one or more friction modifiers; B) from 0.01 to 10 wt %, based on total weight of the lubricating oil composition, of one or more antioxidants; C) from 0.01 to 5 wt %, based on total weight of the lubricating oil composition, of one or more pour point depressants; D) from 0.001 to 5 wt %, based on total weight of the lubricating oil composition, of one or more anti-foam agents; E) from 0.001 to 10 wt %, based on total weight of the lubricating oil composition, of one or more viscosity modifiers; F) from 0.0 to 5 wt %, based on total weight of the lubricating oil composition, of one or more inhibitors and/or anti-rust agents; and/or G) from 0.001 to 10 wt %, based on total weight of the lubricating oil composition, of one or more antiwear agents.

12. The lubricating oil composition of clauses 7-11, wherein the one or more detergents comprise one or more oil-soluble neutral or overbased sulfonates, phenates, sulfurized phenates, thiophosphonates, salicylates, naphthenates, and other oil-soluble carboxylates of an alkali or alkaline earth metal.

13. The lubricating oil composition of clauses 7-12, wherein the one or more dispersants comprise one or more borated or unborated poly(alkenyl)succinimides, wherein the polyalkyenyl is derived from polyisobutylene and the imide is derived from a polyamine.

14. A method of lubricating an internal combustion engine during operation of the engine comprising: (i) providing to a crankcase of the internal combustion engine the lubricating oil composition of clauses 7-13; (ii) providing a fuel in the internal combustion engine; and (iii) combusting the fuel in the internal combustion engine.

15. The method of clause 14, wherein the fuel is one or more of hydrocarbon fuel, renewable fuel, hydrogen fuel, or any blend thereof.

16. The method of clauses 14-15, wherein the engine is a diesel engine.

The invention will now be described by way of non-limiting examples only.

### Examples

### Testing Procedures

Gel permeation chromatography (GPC) samples were prepared by dissolving a sample of either the crude reaction mixture or the isolated polymer product, in tetrahydrofuran (THF) targeting a final sample concentration between 1.0-5.0 mg polymer/ml THF. GPC was run in isocratic mode using stabilized THF as the mobile phase. All polymer Mp, Mn, Mw, Mz values are reported vs. PS standard, unless specified otherwise. All molecular weights are peak average molecular weights (Mp) reported in kDa/mol, as determined by gel permeation chromatography using polystyrene standards, unless otherwise noted. Molecular weight values are reported for both pre-hydrogenation and post-hydrogenation polymers. “A.I.”, “ai”, “a.i.”, and “ai” are wt % active ingredient, unless otherwise indicated.

KV100 is Kinematic viscosity measured at 100° C. according to ASTM D445-19a.

Phosphorus, Boron, Calcium, Zinc, Molybdenum, Magnesium, and Sulfur content are measured by ASTM D5185.

High Temperature High Shear Viscosity, (“HTHS” or “HTHS150”) is determined at 150° C. according to ASTM D4683 and is reported in cPs.

Cold Cranking Simulator (“CCS”), at −25° C. unless otherwise indicated, is a measure of the cold-cranking characteristics of crankcase lubricants and is determined as described in ASTM D5293-92.

Cummins ISB Engine Test. Valve train wear protection was determined according to the Cummins ISB engine test, ASTM D7484-21 in a 5.9 L 6-cyl diesel engine equipped with exhaust gas recirculation. The Cummins ISB test is a two-stage test. Stage A, for 100 hours, the engine was operated with retarded fuel-injection timing to generate excess soot according to the ASTM Protocol. During Stage B, for 250 hours, the engine was operated at cyclic conditions according to the ASTM Protocol to induce valve-train wear. Oil performance was determined by assessing crosshead weight loss (mg) measured as detailed in Section 8.1.5 in ASTM D7484-21, tappet weight loss (mg) measured as detailed in section 8.1.6 in ASTM D7484-21, and camshaft wear (pm) averaged over the 12 lobes measured with a Mitutoyo Snap Gauge and Mitutoyo Digital Indicator as detailed in Section 8.1.7 in ASTM D7484-21.

### Materials

### Polymer Examples

The following ADAMS-isoprene copolymer samples were prepared according to the general methods in the detailed description above.

| | | | | | | GPC Mp | GPC Mp |
|---|---|---|---|---|---|---|---|
| | | | | | | (kDa, vs. PS | (kDa, vs. PS |
| Polymer | | Co- | | ADAMS | Hydrogenation | standard, | standard, |
| Example | ADAMS monomer | monomer | Architecture | wt % | % | pre-hydro.) | post-hydro.) |
| 1 | 1-(N-morpholinyl)-3- | Isoprene | distributed | 4.9% | 87% | 42.1 | 45.1 |
| | phenylbut-3-ene | | | | | | |
| 2 | 1-(N-morpholinyl)-3- | Isoprene | distributed | 4.4% | 92% | 38.3 | 41.2 |
| | phenylbut-3-ene | | | | | | |
| 3 | 1-(4-methyl-1- | Isoprene | distributed | 5.0% | 95% | 38.6 | 41.0 |
| | piperazinyl)-3- | | | | | | |
| | phenylbut-3-ene | | | | | | |
| 4 | 1-(4-methyl-1- | Isoprene | distributed | 10.0% | 88% | 24.0 | 26.5 |
| | piperazinyl)-3- | | | | | | |
| | phenylbut-3-ene | | | | | | |
| 5 | 1-benzylmethylamino- | Isoprene | tetrablock | 10.7% | 99% | 46.9 | 54.2 |
| | 3-phenylbut-3-ene | | | | | | |
| 6 | 1-benzylmethylamino- | Isoprene | tetrablock | 18.2% | 90% | 52.7 | 64.0 |
| | 3-phenylbut-3-ene | | | | | | |
| 7 | 1-benzylphenylamino- | Isoprene | star (per arm) | 6.0% | 85% | 25.6 | |
| | 3-phenylbut-3-ene | | star (total) | 6.0% | 85% | 150.0 | 153.0 |
| 8 | 1-benzylmethylamino- | Isoprene | tetrablock | 12.0% | 87% | 46.0 | 53.7 |
| | 3-phenylbut-3-ene | | | | | | |
| 9 | 1-benzylmethylamino- | Isoprene | tetrablock | 12.0% | 99.5% | 46.0 | 52.3 |
| | 3-phenylbut-3-ene | | | | | | |
| 10 | 1-benzylmethylamino- | Isoprene | diblock | 12.0% | 92% | 45.8 | 55.8 |
| | 3-phenylbut-3-ene | | | | | | |
| 11 | 1-benzylmethylamino- | Isoprene | diblock | 12.0% | 99.5% | 45.8 | 56.3 |
| | 3-phenylbut-3-ene | | | | | | |

### Component Chart

| Short Name | Description |
|---|---|
| Poly(ADAMS/I)-1 | The Hydrogenated ADAMS-Isoprene copolymer of example 3 in 50 wt % |
| | oil grp. III base oil KV₁₀₀ 4 cSt |
| Poly(ADAMS/I)-2 | The Hydrogenated ADAMS-Isoprene copolymer of example 4 in 50 wt % |
| | oil grp. III base oil KV₁₀₀ 4 cSt |
| Poly(ADAMS/I)-3 | The isolated Hydrogenated ADAMS-Isoprene copolymer of example 8 in |
| | 81% grp. III base oil KV₁₀₀ 4 cSt |
| Poly(ADAMS/I)-4 | The isolated Hydrogenated ADAMS-Isoprene copolymer of example 9 in |
| | 81% grp. III base oil KV₁₀₀ 4 cSt |
| Poly(ADAMS/I)-5 | The isolated Hydrogenated ADAMS-Isoprene copolymer of example 10 |
| | in 77% grp. III base oil KV₁₀₀ 4 cSt |
| Poly(ADAMS/I)-6 | The isolated Hydrogenated ADAMS-Isoprene copolymer of example 11 |
| | in 87% grp. III base oil KV₁₀₀ 4 cSt |
| PIBSA-PAM 950 | PIBSA-PAM dispersant (950 Mn PIB) in~51% oil |
| Mn | |
| B-PIBSA-PAM- | Borated PIBSA-PAM dispersant (950 Mn PIB) in~50% oil |
| 950 | |
| PIBSA-PAM 2200 | PIBSA-PAM dispersant (2200 Mn PIB) in~38-43% oil |
| Mn | |
| PIBSA-ester | PIBSA ester of hydrocarbyl-bridged naphthyloxy alcohol in~60% oil. |
| Dispersant | See U.S. 7,485,603. |
| Ca sulfonate | Ca sulfonate detergent (300 TBN, in~45 wt % oil) |
| Mg sulfonate | Mg sulfonate detergent (400 TBN, in~43 wt % oil) |
| Mo friction | Trimeric MoDTC AO/FM (ai~45) |
| modifier | |
| ZDDP | ZDDP (derived from iso-octanol/2-butanol [15/85]), ai~75) |
| DPA antioxidant | Diphenylamine Antioxidant |
| Sulfurized FAME | Sulfurized Fatty Acid Ester |
| Anti-foamant | PDMS silicone anti-foamant |
| PIBSA | PIBSA (Mn 950, ai~72) |
| PIB | Polyisobutylene having an Mn of 950 g/mol |
| Diluent | Group I base oil diluent |
| Lube oil flow | C₁₂₋₁₈ dialkylfumarate/vinyl acetate copolymer in 43.5% diluent available |
| improver (LOFI) | as Infineum V387 ™ from Infineum USA LP, Linden NJ, USA |
| OCP-VM-1 | Ethylene-propylene copolymer viscosity modifier, ai~12.5, available |
| | from Lubrizol as LZ7077 ™ |
| OCP-VM-2 | Ethylene-propylene copolymer viscosity modifier, ai~10.15, available |
| | from Chevron Oronite as Paratone 8900E |

### Example 1: Cummins ISB for Valve Train Wear Protection Test

Oil A, Oil B, and Comparative Oil C were prepared and tested for valve train wear protection according to the Cummins ISB engine test described above. The data are reported in Table 1.

TABLE 1

| | | | Comparative |
|---|---|---|---|
| | Oil A | Oil B | Oil C |
| Component/Property | mass % | mass % | mass % |
| Poly(ADAMS/I)-1 | 1.28 | | |
| Poly(ADAMS/I)-2 | | 1.28 | |
| PIBSA-PAM 950 Mn | 4.00 | 4.00 | |
| B-PIBSA-PAM-950 | 0.50 | 0.50 | |
| PIBSA-PAM 2200 Mn | 4.00 | 4.00 | 7.00 |
| PIBSA-ester Dispersant | | | 1.00 |
| Ca sulfonate | 1.025 | 1.025 | 0.90 |
| Mg sulfonate | 0.85 | 0.85 | 1.10 |
| Mo friction modifier | | | 0.091 |
| ZDDP | 1.00 | 1.00 | 1.00 |
| DPA antioxidant | 2.10 | 2.10 | 0.50 |
| Sulfurized FAME | 0.56 | 0.56 | 0.784 |
| PIBSA | 0.10 | 0.10 | |
| PIB | 0.30 | 0.30 | |
| Anti-foamant | 0.005 | 0.005 | 0.002 |
| Diluent | 2.26 | 2.26 | 2.607 |
| Lube oil flow improver (LOFI) | 0.20 | 0.20 | 0.30 |
| OCP-VM-1 | | | 3.00 |
| OCP-VM-2 | 1.40 | 1.40 | |
| Group II base oil KV₁₀₀ 4 cSt | 48.00 | 48.00 | 41.00 |
| Group II base oil KV₁₀₀ 6 cSt | 32.42 | 32.42 | 40.716 |
| SAE Viscosity Grade | 10W-30 | 10W-30 | 10W-30 |
| KV100 | 9.69 | 9.55 | 10.19 |
| HTHS150 | 3.04 | 3.03 | 3.18 |
| CCS @−25° C. | 6331 | 6157 | 6310 |
| mass % P | 0.081 | 0.082 | 0.081 |
| mass % Ca | 0.120 | 0.120 | 0.105 |
| mass % Mg | 0.083 | 0.082 | 0.103 |
| mass % S | 0.303 | 0.288 | 0.330 |
| Camshaft Wear (μm) | 18.6 | 25.4 | 50.7 |
| Cummins ISB engine test | | | |
| Tappet Weight Loss (mg) | 57.2 | 59.3 | 77.3 |
| Cummins ISB engine test | | | |
| Crosshead Weight Loss (mg) | 2.8 | 2.3 | 4.7 |
| Cummins ISB engine test | | | |

### Example 2: Soot-Induced Viscosity Control Bench Test

A solution of 2.0 wt % of the selected isolated ADAMS-Isoprene copolymer in Group II base oil (KV₁₀₀ 6 cSt) was prepared by mixing appropriate amounts of the finished component and the base oil, heated to 75° C. for between 1-3 hours, until the polymer was fully dissolved.

A suspension of 9.0 wt % carbon black (Vulcan XC72R) in the ADAMS-isoprene copolymer solution was prepared by weighing 45.5 g of the 2.0 wt % polymer solution added it to a 100 ml beaker containing 4.5 g of Vulcan XC72R carbon black. The suspension was mixed via overhead stirring (200-400 rpm) at 90° C. for 16 h, followed by 100° C. for 1 h under an air atmosphere.

Soot-induced viscosity growth experiments were carried out on a Haake RS600 Rheometer, controlled by Haake RheoWin Job Manager software (ver. 4.30.0028), using the following conditions in Table 2:

TABLE 2
Sooted Rheology Methods

| | |
|---|---|
| Measuring | Z20 DIN |
| Geometry | |

| | | |
|---|---|---|
| Gap | 4.2 | mm |
| Volume | 8.2 | mL |
| Temp. | 100° | C. |

| Shear | | | Time | | |
|---|---|---|---|---|---|
| Profile | Type | Shear (s⁻¹) | (s) | # Data | Settings |
| Start | Rotation | 10.0 | 30 | 2 | Acquisition: logarithmic |
| | Recover | 0 | 600 | 11 | Acquisition: Linear |
| | Rot. | 1.0 → | 480 | 100 | Parameters: linear, continuous |
| | Ramp | 1000.0 | | | Acquisition: logarithmic |
| | Rot. | 1000.0 → | 480 | 100 | Parameters: linear, continuous |
| | Ramp | 1.0 | | | Acquisition: logarithmic |
| End | Rot. | 1.0 → | 480 | 300 | Parameters: linear, continuous |
| | Ramp | 1000.0 | | | Acquisition: logarithmic |

The dynamic viscosity of the sample on the final shear sweep was used to compare the soot dispersancy of the different components diluted to 2.0 wt % of the active polymer in Group III base oil. Viscosities (l, Pa-s) were taken at approximate shear rates of 2.1±0.1, 4.1±0.1, and 8.1±0.1 s⁻¹ as stated in the Table 3 below.

TABLE 3
Soot-Induced Viscosity Growth Data

| | | | | | Comparative |
|---|---|---|---|---|---|
| | Oil C | Oil D | Oil E | Oil F | Oil G |
| Component/Property | mass % | mass % | mass % | mass % | mass % |
| Poly(ADAMS/I)-3 | 10.53 | | | | |
| Poly(ADAMS/I)-4 | | 10.53 | | | |
| Poly(ADAMS/I)-5 | | | 8.70 | | |
| Poly(ADAMS/I)-6 | | | | 15.39 | |
| Group II base oil KV₁₀₀ 6 cSt | 89.47 | 89.47 | 91.3 | 84.61 | 100 |
| η (Pa-s) at shear rate at~2.1 s⁻¹ | 2.33 | 1.73 | 2.59 | 3.41 | 9.43 |
| η (Pa-s) at shear rate at~4.1 s⁻¹ | 1.45 | 1.00 | 1.68 | 2.33 | 6.30 |
| η (Pa-s) at shear rate at~8.1 s⁻¹ | 0.82 | 0.58 | 0.96 | 1.29 | 4.52 |

All documents described herein are incorporated by reference herein, including any priority documents and/or testing procedures, to the extent they are not inconsistent with this text. As should be apparent from the foregoing general description and the specific embodiments, while forms of the invention have been illustrated and described, various modifications can be made without departing from the spirit and scope of the invention. Accordingly, it is not intended that the invention be limited thereby. The term “comprising” specifies the presence of stated features, steps, integers, or components, but does not preclude the presence or addition of one or more other features, steps, integers, components, or groups thereof. As such, the term “comprising” is considered essentially synonymous with the term “including.” Similarly, whenever a composition, an element, or a group of elements is preceded with the transitional phrase “comprising,” it should be understood that the same composition or group of elements is contemplated with transitional phrases “consisting essentially of,” “consisting of,” “selected from the group of consisting of,” or “can be”/“may be”/“is” preceding the recitation of the composition, element, or elements, and vice versa. In juxtaposition to the well-known terms “comprising” meaning “including what follows and anything else” [open] and “consisting of” meaning “including only what follows” [closed], the term “consisting essentially of” should be understood to be semi-inclusive and to mean, in accordance with US judicial interpretation, including that which follows and other things that do not materially affect the basic and novel properties.

Applicants have attempted to disclose all embodiments and applications of the disclosed subject matter that could be reasonably foreseen. However, there may be unforeseeable, insubstantial modifications that remain as equivalents. While the present invention has been described in conjunction with specific, exemplary embodiments thereof, it is evident that many alterations, modifications, and variations will be apparent to those skilled in the art in light of the foregoing description without departing from the spirit or scope of the present disclosure. Accordingly, the present disclosure is intended to embrace all such alterations, modifications, and variations of the above detailed description.

All patents, test procedures, and other documents cited herein, including priority documents, are fully incorporated by reference to the extent such disclosure is not inconsistent with this invention and for all jurisdictions in which such incorporation is permitted.

When numerical lower limits and numerical upper limits are listed herein, ranges from any lower limit to any upper limit are contemplated.

1. A copolymer comprising:
(a) 10.0 to 20.0 wt % of amine-derivatized alpha-methyl styrene (ADAMS) repeat units according to structure (I): 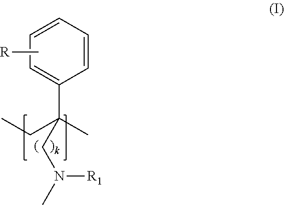
wherein:
k is an integer from 1 to 3;
R₁ a hydrogen or a benzyl group
R is hydrogen, a phenyl ring co-attached at two neighboring ring carbon positions with the phenyl ring shown so as to form a naphthalene assembly, a phenyl group attached at a single carbon of the phenyl ring shown, a C1-C4 hydrocarbyl group, a C1-C6 hydrocarbyl group containing 1 to 4 additional heteroatoms selected from the group consisting of O, N, S, P, Se, and combinations thereof, and
(b) the remaining 80.0 to 90.0 wt % of repeat units corresponding to the reacted form of isoprene, and wherein the peak-average molecular weight of the copolymer is between 45.0 to 65.0 kDa.

2. The copolymer of claim 1, wherein the copolymer is partially or substantially hydrogenated.

3. The copolymer of claim 1, wherein k=2.

4. The copolymer of claim 1, further comprising an alkyl residue from a monofunctional initiator selected from the group consisting of alkyl lithium, alkyl sodium, alkyl potassium and combinations thereof, and present at one or more termini of a polymer backbone.

5. The copolymer of claim 4, wherein the alkyl residues from the monofunctional initiator comprise methyl, ethyl, n-propyl, isopropyl, n-butyl, iso-butyl, sec-butyl, tert-butyl, n-amyl, iso-amyl, sec-amyl, tert-amyl, hexyl groups, or combinations thereof.

6. The copolymer of claim 1, wherein one or more polymer blocks of the copolymer form a distributed polymer architecture, a diblock, a triblock, a tetrablock, a pentablock, a hexablock, a star polymer architecture, or combinations thereof.

7. A copolymer comprising:
(a) 5.0 to 10.0 wt % of amine-derivatized alpha-methyl styrene (ADAMS) repeat units according to structure (II): 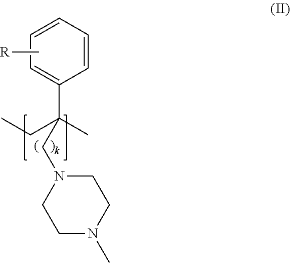
wherein:
k is an integer from 1 to 3;
R is hydrogen, a phenyl ring co-attached at two neighboring ring carbon positions with the phenyl ring shown so as to form a naphthalene assembly, a phenyl group attached at a single carbon of the phenyl ring shown, a C1-C4 hydrocarbyl group, a C1-C6 hydrocarbyl group containing 1 to 4 additional heteroatoms selected from the group consisting of O, N, S, P, Se, and combinations thereof, and
(b) the remaining 90.0 to 95.0 wt % of repeat units corresponding to the reacted form of isoprene, and
wherein, the peak-average molecular weight of the copolymer is between 24.0 to 42.0 kDa.

8. The copolymer of claim 7, wherein the copolymer is partially or substantially hydrogenated.

9. The copolymer of claim 7, wherein k=2.

10. The copolymer of claim 7, further comprising an alkyl residue from a monofunctional initiator selected from alkyl lithium, alkyl sodium, alkyl potassium and combinations thereof, and present at one or more termini of a polymer backbone.

11. The copolymer of claim 10, wherein the alkyl residues from the monofunctional initiator comprise methyl, ethyl, n-propyl, isopropyl, n-butyl, iso-butyl, sec-butyl, tert-butyl, n-amyl, iso-amyl, sec-amyl, tert-amyl, hexyl groups, or combinations thereof.

12. The copolymer of claim 7, wherein one or more polymer blocks of the copolymer form a distributed polymer architecture, a diblock, a triblock, a tetrablock, a pentablock, a hexablock, a star polymer architecture, or combinations thereof.

13. A copolymer comprising:
(a) 4.0 to 6.0 wt % of amine-derivatized alpha-methyl styrene (ADAMS) repeat units according to structure (III): 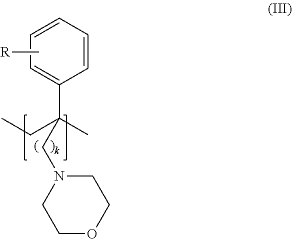
wherein:
k is an integer from 1 to 3;
R is hydrogen, a phenyl ring co-attached at two neighboring ring carbon positions with the phenyl ring shown so as to form a naphthalene assembly, a phenyl group attached at a single carbon of the phenyl ring shown, a C1-C4 hydrocarbyl group, a C1-C6 hydrocarbyl group containing 1 to 4 additional heteroatoms selected from the group consisting of O, N, S, P, Se, and combinations thereof,
(b) the remaining 94.0 to 96.0 wt % of repeat units corresponding to the reacted form of isoprene, and
wherein, the peak-average molecular weight of the copolymer is between 36.0 to 46.0 kDa.

14. The copolymer of claim 13, wherein the copolymer is partially or substantially hydrogenated.

15. The copolymer of claim 13, wherein k=2.

16. The copolymer of claim 13, further comprising an alkyl residue from a monofunctional initiator selected from the group consisting of alkyl lithium, alkyl sodium, alkyl potassium initiator and combinations thereof, and present at one or more termini of a polymer backbone.

17. The copolymer of claim 16, wherein the alkyl residues from the monofunctional initiator comprise methyl, ethyl, n-propyl, isopropyl, n-butyl, iso-butyl, sec-butyl, tert-butyl, n-amyl, iso-amyl, sec-amyl, tert-amyl, hexyl groups, or combinations thereof.

18. The copolymer of claim 13, wherein one or more polymer blocks of the copolymer form a distributed polymer architecture, a diblock, a triblock, a tetrablock, a pentablock, a hexablock, a star polymer architecture, or combinations thereof.

19. A copolymer comprising:
(a) one or more amine-derivatized alpha-methyl styrene (ADAMS) repeat units according to structure (IV): 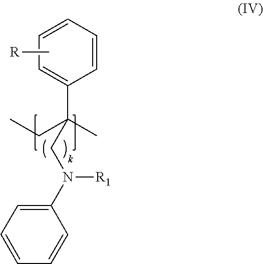
wherein:
k is an integer from 1 to 3;
R₁ a hydrogen or a benzyl group
R is hydrogen, a phenyl ring co-attached at two neighboring ring carbon positions with the phenyl ring shown so as to form a naphthalene assembly, a phenyl group attached at a single carbon of the phenyl ring shown, a C1-C4 hydrocarbyl group, a C1-C6 hydrocarbyl group containing 1 to 4 additional heteroatoms selected from the group consisting of O, N, S, P, Se, and combinations thereof, and
(b) the remaining repeat units corresponding to the reacted form of isoprene.

20. The copolymer of claim 19, wherein the copolymer is partially or substantially hydrogenated.

21. The copolymer of claim 19, wherein k=2.

22. The copolymer of claim 19, further comprising an alkyl residue from a monofunctional initiator selected from the group consisting of alkyl lithium, alkyl sodium, alkyl potassium, and combinations thereof, and present at one or more termini of a polymer backbone.

23. The copolymer of claim 22, wherein the alkyl residues from the monofunctional initiator comprise methyl, ethyl, n-propyl, isopropyl, n-butyl, iso-butyl, sec-butyl, tert-butyl, n-amyl, iso-amyl, sec-amyl, tert-amyl, hexyl groups, or combinations thereof.

24. The copolymer of claim 19, wherein one or more polymer blocks of the copolymer form a distributed polymer architecture, a diblock, a triblock, a tetrablock, a pentablock, a hexablock, a star polymer architecture, or combinations thereof.

25. A lubricating oil composition comprising or resulting form the admixing of:
(i) at least 50 wt % of one or more base oils, based upon the weight of the lubricating oil composition;
(ii) one or more dispersants;
(iii) one or more detergents; and
(iv) one or more copolymers of claim 1.

26. The lubricating oil composition of claim 25, wherein the composition has an SAE viscosity grade of 20 W-X, 15 W-X, 10 W-X, 5 W-X, or OW-X, where X represents any one of 8, 12, 16, 20, 30, 40, or 50.

27. The lubricating oil composition of claim 25, comprising or resulting from the admixing of:
(i) from 50 to 99 mass % of the one or more of the base oils, based upon the weight of the lubricating oil composition;
(ii) from 0.01 to 20 wt % based on total weight of the lubricating oil composition, of the one or more dispersants;
(iii) from 0.10 to 20 mass %, based upon the weight of the lubricating oil composition, of the one or more detergents; and
(iv) from 0.10 to 20 mass %, based upon the weight of the lubricating oil composition, of the one or more copolymers.

28. The lubricating oil composition of claim 25, further comprising one, two, three, four, five, six or more of additional additives selected from the group consisting of: friction modifiers; antioxidants; pour point depressants; anti-foam agents; viscosity modifiers; corrosion inhibitors and/or anti-rust agents; and antiwear agents.

29. The lubricating oil composition of claim 25, further comprising one, two, three, four, five, six, or more of:
A) from 0.01 to 5 wt %, based on total weight of the lubricating oil composition, of one or more friction modifiers;
B) from 0.01 to 10 wt %, based on total weight of the lubricating oil composition, of one or more antioxidants;
C) from 0.01 to 5 wt %, based on total weight of the lubricating oil composition, of one or more pour point depressants;
D) from 0.001 to 5 wt %, based on total weight of the lubricating oil composition, of one or more anti-foam agents;
E) from 0.001 to 10 wt %, based on total weight of the lubricating oil composition, of one or more viscosity modifiers;
F) from 0.0 to 5 wt %, based on total weight of the lubricating oil composition, of one or more inhibitors and/or anti-rust agents; and/or
G) from 0.001 to 10 wt %, based on total weight of the lubricating oil composition, of one or more antiwear agents.

30. The lubricating oil composition of claim 25, wherein the one or more detergents comprise one or more oil-soluble neutral or overbased sulfonates, phenates, sulfurized phenates, thiophosphonates, salicylates, naphthenates, and other oil-soluble carboxylates of an alkali or alkaline earth metal.

31. The lubricating oil composition of claim 25, wherein the one or more dispersants comprise one or more borated or unborated poly(alkenyl)succinimides, wherein the polyalkyenyl is derived from polyisobutylene and the imide is derived from a polyamine.

32. A method of lubricating an internal combustion engine during operation of the engine comprising:
(i) providing to a crankcase of the internal combustion engine the lubricating oil composition of claim 25;
(ii) providing a fuel in the internal combustion engine; and
(iii) combusting the fuel in the internal combustion engine.

33. The method of claim 32, wherein the fuel is one or more of hydrocarbon fuel, renewable fuel, hydrogen fuel, or any blend thereof.

34. The method of claim 32, wherein the engine is a diesel engine.

35. A lubricating oil composition comprising or resulting form the admixing of:
(i) at least 50 wt % of one or more base oils, based upon the weight of the lubricating oil composition;
(ii) one or more dispersants;
(iii) one or more detergents; and
(iv) one or more copolymers of claim 7.

36. The lubricating oil composition of claim 35, wherein the composition has an SAE viscosity grade of 20 W-X, 15 W-X, 10 W-X, 5 W-X, or OW-X, where X represents any one of 8, 12, 16, 20, 30, 40, or 50.

37. The lubricating oil composition of claim 35, comprising or resulting from the admixing of:
(i) from 50 to 99 mass % of the one or more of the base oils, based upon the weight of the lubricating oil composition;
(ii) from 0.01 to 20 wt % based on total weight of the lubricating oil composition, of the one or more dispersants;
(iii) from 0.10 to 20 mass %, based upon the weight of the lubricating oil composition, of the one or more detergents; and
(iv) from 0.10 to 20 mass %, based upon the weight of the lubricating oil composition, of the one or more copolymers.

38. The lubricating oil composition of claim 35, further comprising one, two, three, four, five, six or more of additional additives selected from the group consisting of: friction modifiers; antioxidants; pour point depressants; anti-foam agents; viscosity modifiers; corrosion inhibitors and/or anti-rust agents; and antiwear agents.

39. The lubricating oil composition of claim 35, further comprising one, two, three, four, five, six, or more of:
A) from 0.01 to 5 wt %, based on total weight of the lubricating oil composition, of one or more friction modifiers;
B) from 0.01 to 10 wt %, based on total weight of the lubricating oil composition, of one or more antioxidants;
C) from 0.01 to 5 wt %, based on total weight of the lubricating oil composition, of one or more pour point depressants;
D) from 0.001 to 5 wt %, based on total weight of the lubricating oil composition, of one or more anti-foam agents;
E) from 0.001 to 10 wt %, based on total weight of the lubricating oil composition, of one or more viscosity modifiers;
F) from 0.0 to 5 wt %, based on total weight of the lubricating oil composition, of one or more inhibitors and/or anti-rust agents; and/or
G) from 0.001 to 10 wt %, based on total weight of the lubricating oil composition, of one or more antiwear agents.

40. The lubricating oil composition of claim 35, wherein the one or more detergents comprise one or more oil-soluble neutral or overbased sulfonates, phenates, sulfurized phenates, thiophosphonates, salicylates, naphthenates, and other oil-soluble carboxylates of an alkali or alkaline earth metal.

41. The lubricating oil composition of claim 35, wherein the one or more dispersants comprise one or more borated or unborated poly(alkenyl)succinimides, wherein the polyalkyenyl is derived from polyisobutylene and the imide is derived from a polyamine.

42. A method of lubricating an internal combustion engine during operation of the engine comprising:
(i) providing to a crankcase of the internal combustion engine the lubricating oil composition of claim 35;
(ii) providing a fuel in the internal combustion engine; and
(iii) combusting the fuel in the internal combustion engine.

43. The method of claim 42, wherein the fuel is one or more of hydrocarbon fuel, renewable fuel, hydrogen fuel, or any blend thereof.

44. The method of claim 42, wherein the engine is a diesel engine.

45. A lubricating oil composition comprising or resulting form the admixing of:
(i) at least 50 wt % of one or more base oils, based upon the weight of the lubricating oil composition;
(ii) one or more dispersants;
(iii) one or more detergents; and
(iv) one or more copolymers of claim 13.

46. The lubricating oil composition of claim 45, where the composition has an SAE viscosity grade of 20 W-X, 15 W-X, 10 W-X, 5 W-X, or OW-X, where X represents any one of 8, 12, 16, 20, 30, 40, and 50.

47. The lubricating oil composition of claim 45, comprising or resulting from the admixing of:
(i) from 50 to 99 mass % of the one or more of the base oils, based upon the weight of the lubricating oil composition;
(ii) from 0.01 to 20 wt % based on total weight of the lubricating oil composition, of the one or more dispersants;
(iii) from 0.10 to 20 mass %, based upon the weight of the lubricating oil composition, of the one or more detergents; and
(iv) from 0.10 to 20 mass %, based upon the weight of the lubricating oil composition, of the one or more functionalized polymers.

48. The lubricating oil composition of claim 45, further comprising one, two, three, four, five, six or more of additional additives selected from the group consisting of: friction modifiers; antioxidants; pour point depressants; anti-foam agents; viscosity modifiers; corrosion inhibitors and/or anti-rust agents; and antiwear agents.

49. The lubricating oil composition of claim 45, further comprising one, two, three, four, five, six, or more of:
A) from 0.01 to 5 wt %, based on total weight of the lubricating oil composition, of one or more friction modifiers;
B) from 0.01 to 10 wt %, based on total weight of the lubricating oil composition, of one or more antioxidants;
C) from 0.01 to 5 wt %, based on total weight of the lubricating oil composition, of one or more pour point depressants;
D) from 0.001 to 5 wt %, based on total weight of the lubricating oil composition, of one or more anti-foam agents;
E) from 0.001 to 10 wt %, based on total weight of the lubricating oil composition, of one or more viscosity modifiers;
F) from 0.0 to 5 wt %, based on total weight of the lubricating oil composition, of one or more inhibitors and/or anti-rust agents; and/or
G) from 0.001 to 10 wt %, based on total weight of the lubricating oil composition, of one or more antiwear agents.

50. The lubricating oil composition of claim 45, wherein the one or more detergents comprise one or more oil-soluble neutral or overbased sulfonates, phenates, sulfurized phenates, thiophosphonates, salicylates, naphthenates, and other oil-soluble carboxylates of a alkali or alkaline earth metal.

51. The lubricating oil composition of claim 45, wherein the one or more dispersants comprise one or more borated or unborated poly(alkenyl)succinimides, where the polyalkyenyl is derived from polyisobutylene and the imide is derived from a polyamine.

52. A method of lubricating an internal combustion engine during operation of the engine comprising:
(i) providing to a crankcase of the internal combustion engine the lubricating oil composition of claim 45;
(ii) providing a fuel in the internal combustion engine; and
(iii) combusting the fuel in the internal combustion engine.

53. The method of claim 52, wherein the fuel is one or more of hydrocarbon fuel, renewable fuel, hydrogen fuel, or any blend thereof.

54. The method of claim 52, wherein the engine is a diesel engine.

55. A lubricating oil composition comprising or resulting form the admixing of:
(i) at least 50 wt % of one or more base oils, based upon the weight of the lubricating oil composition;
(ii) one or more dispersants;
(iii) one or more detergents; and
(iv) one or more copolymers of claim 19.

56. The lubricating oil composition of claim 55, where the composition has an SAE viscosity grade of 20 W-X, 15 W-X, 10 W-X, 5 W-X, or OW-X, where X represents any one of 8, 12, 16, 20, 30, 40, and 50.

57. The lubricating oil composition of claim 55, comprising or resulting from the admixing of:
(i) from 50 to 99 mass % of the one or more of the base oils, based upon the weight of the lubricating oil composition;
(ii) from 0.01 to 20 wt % based on total weight of the lubricating oil composition, of the one or more dispersants;
(iii) from 0.10 to 20 mass %, based upon the weight of the lubricating oil composition, of the one or more detergents; and
(iv) from 0.10 to 20 mass %, based upon the weight of the lubricating oil composition, of the one or more copolymers.

58. The lubricating oil composition of claim 55, further comprising one, two, three, four, five, six or more of additional additives selected from the group consisting of: friction modifiers; antioxidants; pour point depressants; anti-foam agents; viscosity modifiers; corrosion inhibitors and/or anti-rust agents; and antiwear agents.

59. The lubricating oil composition of claim 55, further comprising one, two, three, four, five, six, or more of:
A) from 0.01 to 5 wt %, based on total weight of the lubricating oil composition, of one or more friction modifiers;
B) from 0.01 to 10 wt %, based on total weight of the lubricating oil composition, of one or more antioxidants;
C) from 0.01 to 5 wt %, based on total weight of the lubricating oil composition, of one or more pour point depressants;
D) from 0.001 to 5 wt %, based on total weight of the lubricating oil composition, of one or more anti-foam agents;
E) from 0.001 to 10 wt %, based on total weight of the lubricating oil composition, of one or more viscosity modifiers;
F) from 0.0 to 5 wt %, based on total weight of the lubricating oil composition, of one or more inhibitors and/or anti-rust agents; and/or
G) from 0.001 to 10 wt %, based on total weight of the lubricating oil composition, of one or more antiwear agents.

60. The lubricating oil composition of claim 55, wherein the one or more detergents comprise one or more oil-soluble neutral or overbased sulfonates, phenates, sulfurized phenates, thiophosphonates, salicylates, naphthenates, and other oil-soluble carboxylates of a alkali or alkaline earth metal.

61. The lubricating oil composition of claim 55, wherein the one or more dispersants comprise one or more borated or unborated poly(alkenyl)succinimides, wherein the polyalkyenyl is derived from polyisobutylene and the imide is derived from a polyamine.

62. A method of lubricating an internal combustion engine during operation of the engine comprising:
(i) providing to a crankcase of the internal combustion engine the lubricating composition of claim 55;
(ii) providing a fuel in the internal combustion engine; and
(iii) combusting the fuel in the internal combustion engine.

63. The method of claim 62, wherein the fuel is one or more of hydrocarbon fuel, renewable fuel, hydrogen fuel, or any blend thereof.

64. The method of claim 62, wherein the engine is a diesel engine.
